(19) United States
(12) Patent Application Publication
(10) Pub. No.: US 2026/0209248 A1
(43) Pub. Date: Jul. 23, 2026

(54) INHIBITORS OF KIF18A AND USES THEREOF

(71) Applicant: Insilico Medicine IP Limited, Hong Kong (CN)

(72) Inventors: Xiao DING, Shanghai (CN); Feng REN, Shanghai (CN); Min ZHENG, Shanghai (CN); Wei ZHU, Shanghai (CN)

(21) Appl. No.: 19/140,581

(22) PCT Filed: Dec. 27, 2023
(86) PCT No.: PCT/CN2023/142332

§ 371 Date: Jun. 18, 2025

(30) Foreign Application Priority Data
Dec. 28, 2022 (WO) PCT/CN2022/142885

(51) Int. Cl. C07D 498/14; A61K 31/438; A61K 31/444; A61K 31/4545; A61K 31/501; A61K 31/506; A61K 31/5377; A61K 31/5383; A61K 31/553; C07D 401/04; C07D 401/12; C07D 401/14; C07D 405/14; C07D 498/04

(52) U.S. Cl. CPC C07D 498/14; A61K 31/438; A61K 31/444; A61K 31/4545; A61K 31/501; A61K 31/506; A61K 31/5377; A61K 31/5383; A61K 31/553; C07D 401/04; C07D 401/12; C07D 401/14; C07D 405/14; C07D 498/04

(57) ABSTRACT

Described herein are KIF18A inhibitors and pharmaceutical compositions comprising said inhibitors. The subject compounds and compositions are useful for the treatment of a disease or disorder associated with KIF18A.

### CROSS REFERENCE TO RELATED APPLICATIONS

This application claims the benefit of PCT International Application No. PCT/CN2022/142885 filed on Dec. 28, 2022, which application is incorporated herein by reference in its entirety.

### BACKGROUND

The KIF18A gene belongs to the Kinesin-8 subfamily and is a plus-end-directed motor. KIF18A is believed to influence dynamics at the plus end of kinetochore microtubules to control correct chromosome positioning and spindle tension. Depletion of human KIF18A leads to longer spindles, increased chromosome oscillation at metaphase, and activation of the mitotic spindle assembly checkpoint in HeLa cervical cancer cells (MI Mayr et al, Current Biology 17, 488-98, 2007). KIF18A is a viable target for the treatment of cancer. KIF18A is overexpressed in various types of cancers, including but not limited to colon, breast, lung, pancreas, prostate, bladder, head, neck, cervix, and ovarian cancers. Further, genetic deletion or knockdown, or inhibition of KIF18A effects mitotic spindle apparatus in cancer cell lines. Particularly, inhibition of KIF18A has been found to induce mitotic cell arrest, a known vulnerability that can promote cell death in mitosis via apoptosis, mitotic catastrophe, or multipolarity driven lethality or death after mitotic slippage in interphase. Accordingly, there has been a strong interest in finding inhibitors of KIF18A proteins.

Thus, the inhibition of KIF18A ATPase activity is a promising approach for the development of novel anti-cancer agents.

### SUMMARY

Disclosed herein is a compound of Formula (I), (II), or a pharmaceutically acceptable salt thereof:

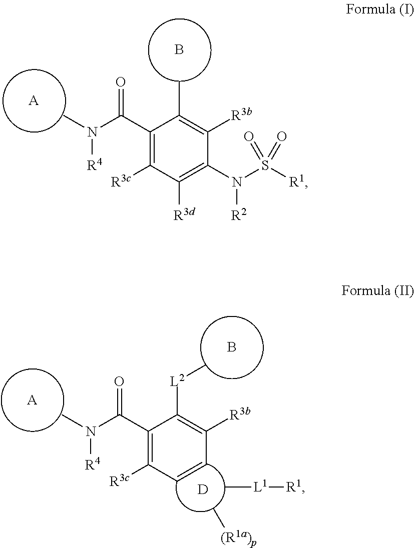

each as disclosed herein.

In some embodiments, disclosed herein is a method of modulating kinase-like protein 18A (KIF18A) in a subject in need thereof, comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc), or a pharmaceutically acceptable salt thereof. In some embodiments, disclosed herein is a method of inhibiting kinase-like protein 18A (KIF18A) in a subject in need thereof, comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc), or a pharmaceutically acceptable salt thereof.

In some embodiments, disclosed herein is a method of treating cancer in a mammal in need thereof, comprising administering to the mammal a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc), or a pharmaceutically acceptable salt thereof. In some embodiments, disclosed herein is a method of treating cancer in a mammal in need thereof, comprising administering to the mammal a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc), or a pharmaceutically acceptable salt thereof, wherein the cancer is selected from the group consisting of (a) a solid or hematologically derived tumor selected from cancer of the bladder, endometrial, lung squamous cell, breast, colon, kidney, liver, lung, small cell lung cancer, esophagus, gallbladder, brain, head and neck, ovary, pancreas, stomach, cervix, thyroid, prostate, and skin; (b) a hematopoietic tumor of lymphoid lineage selected from leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, and Burkett's lymphoma; (c) a hematopoietic tumor of myeloid lineage selected from acute and chronic myelogenous leukemias, myelodysplastic syndrome and promyelocytic leukemia; (D) a tumor of mesenchymal origin selected from fibrosarcoma and rhabdomyosarcoma; € a tumor of the central and peripheral nervous system selected from astrocytoma, neuroblastoma, glioma, and schwannoma; and (f) a melanoma, seminoma, teratocarcinoma, osteosarcoma, xenoderoma pigmentosum, keratoctanthoma, thyroid follicular cancer or Karposi's sarcoma.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)=CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₈ fully saturated cycloalkyl or C₃-C₈ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₅ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₇ heterocycloalkenyl), from two to five carbon atoms (e.g., C₂-C₈ fully saturated heterocycloalkyl or C₂-C₈ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

### Compounds

The compounds of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc), described herein, or a pharmaceutically acceptable salt thereof, are KIF18A inhibitors and are useful in the treatment of a disease or disorder associated with KIF18A. In some embodiments, the compounds of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc), or a pharmaceutically acceptable salt thereof, are useful in the treatment of cancer. In some embodiments, the cancer is selected from the group consisting of (a) a solid or hematologically derived tumor; (b) a hematopoietic tumor of lymphoid lineage; (c) a hematopoietic tumor of myeloid lineage; (d) a tumor of mesenchymal origin; (e) a tumor of the central and peripheral nervous system; and (f) a melanoma, seminoma, teratocarcinoma, osteosarcoma, xenoderoma pigmentosum, keratoctanthoma, thyroid follicular cancer or Karposi's sarcoma.

In some embodiments disclosed herein is a compound of Formula (I), or a pharmaceutically acceptable salt thereof:

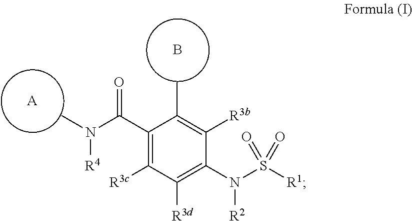

wherein:
R¹ is selected from C₁₋₆alkyl and C₃₋₆cycloalkyl, wherein C₁₋₆alkyl and C₃₋₆cycloalkyl are optionally substituted with one, two, or three groups selected from R^{9a};
R² is selected from hydrogen and C₁₋₆alkyl;
ring B is selected from C₃₋₁₂cycloalkyl, C₂₋₁₄heterocycloalkyl, C₆₋₁₀aryl, and C₁₋₉heteroaryl, wherein C₃₋₁₂cycloalkyl, C₂₋₁₄heterocycloalkyl, C₆₋₁₀aryl, and C₁₋₉heteroaryl are optionally substituted with one, two, or three groups selected from R^{9b};
R^{3b} and R^{3c} are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(=NH)N(R¹⁰)(R¹¹), —S(═O)(=NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9c};
R^{3d} is selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9d}; or R² and R^{3d} are combined to form a C₂₋₉heterocycloalkyl or C₂₋₉heteroaryl, wherein the C₂₋₉heterocycloalkyl and C₂₋₉heteroaryl are optionally substituted with one, two, or three groups selected from R^{9d};
R⁴ is selected from hydrogen and C₁₋₆alkyl;
ring A is a pyridinone ring, a pyridazinone ring, a bicyclic heterocyclic ring, or a tricyclic heterocyclic ring, wherein the pyridinone ring, pyridazinone ring, bicyclic heterocyclic ring, and tricyclic heterocyclic ring are optionally substituted with one or more R^{6a} groups;
each R⁶, is independently selected from halogen, hydroxy, oxo, —CN, C₁₋₆alkyl, C₁₋₆haloalkyl, C₁₋₆alkoxy, C₂₋₆alkenyl, C₂₋₆alkynyl, C₂₋₆cycloalkyl, C₂₋₉heterocycloalkyl, C₆₋₁₀aryl, and C₁₋₉heteroaryl; wherein C₁₋₆alkyl, C₁₋₆haloalkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9e};
each R^{9a}, R^{9b}, R^{9c}, R^{9d}, and R^{9e} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂;
each R¹⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆ haloalkyl, C₁₋₆alkoxy, C₃₋₆cycloalkyl, C₂₋₁₀heterocycloalkyl, C₆₋₁₀aryl, and C₁₋₉heteroaryl; each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl; and each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆ cycloalkyl, C₂₋₁₀heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆ alkoxy, C₃₋₆cycloalkyl, C₂₋₉heterocycloalkyl, C₆₋₁₀aryl, and C₁₋₉heteroaryl.

In some embodiments of a compound of Formula (I), or a pharmaceutically acceptable salt thereof, ring A is a pyridinone ring or a pyridazinone ring, wherein the pyridinone ring and pyridazinone ring are optionally substituted with one or more R^{6a} groups. In some embodiments, ring A is a pyridinone ring optionally substituted with one or more R^{6a} groups. In some embodiments, or a pharmaceutically acceptable salt thereof, ring A is a pyridazinone ring optionally substituted with one or more R^{6a} groups. In some embodiments, each R^{6a} is independently selected from halogen, C₁₋₆alkyl, C₁₋₆haloalkyl, C₃₋₆cycloalkyl, and C₂₋₉heterocycloalkyl; wherein C₁₋₆alkyl, C₁₋₆haloalkyl, C₃₋₆cycloalkyl, and C₂₋₉heterocycloalkyl are optionally substituted with one, two, or three groups selected from R^{9e}.

In some embodiments of a compound of Formula (I), or a pharmaceutically acceptable salt thereof, ring A is a bicyclic heterocyclic ring or a tricyclic heterocyclic ring, wherein the bicyclic heterocyclic ring and tricyclic heterocyclic ring are optionally substituted with one or more R^{6a} groups. In some embodiments, ring A is a bicyclic heterocyclic ring optionally substituted with one or more R^{6a} groups. In some embodiments, ring A is a tricyclic heterocyclic ring optionally substituted with one or more R^{6a} groups. In some embodiments, each R^{6a} is independently selected from halogen, C₁₋₆alkyl, C₁₋₆haloalkyl, C₃₋₆cycloalkyl, and C₂₋₉heterocycloalkyl; wherein C₁₋₆alkyl, C₁₋₆haloalkyl, C₃₋₆cycloalkyl, and C₂₋₉heterocycloalkyl are optionally substituted with one, two, or three groups selected from R^{9c}.

In some embodiments, disclosed herein is a compound of Formula (I), or a pharmaceutically acceptable salt thereof, wherein ring A is a bicyclic heteroaryl ring or a tricyclic heteroaryl ring, wherein the bicyclic heteroaryl ring and tricyclic heteroaryl ring are optionally substituted with one or more R^{6a} groups. In some embodiments, ring A is a bicyclic heteroaryl ring optionally substituted with one or more R^{6a} groups. In some embodiments, ring A is a tricyclic heteroaryl ring optionally substituted with one or more R^{6a} groups. In some embodiments, ring A is an 11-15 membered bicyclic heterocyclic ring or an 11-17 membered tricyclic heterocyclic ring, wherein the 11-15 membered bicyclic heterocyclic ring and 11-17 membered tricyclic heterocyclic ring are optionally substituted with one or more R^{6a} groups. In some embodiments, ring A is an 11-15 membered bicyclic heterocyclic ring optionally substituted with one or more R^{6a} groups. In some embodiments, ring A is an 11-17 membered tricyclic heterocyclic ring optionally substituted with one or more R^{6a} groups. In some embodiments, each R^{6a} is independently selected from halogen, C₁₋₆alkyl, C₁₋₆haloalkyl, C₃₋₆cycloalkyl, and C₂₋₉heterocycloalkyl; wherein C₁₋₆ alkyl, C₁₋₆haloalkyl, C₃₋₆cycloalkyl, and C₂₋₉heterocycloalkyl are optionally substituted with one, two, or three groups selected from R^{9c}. In some embodiments, each R^{6a} is independently selected from halogen, C₁₋₆alkyl, C₁₋₆haloalkyl, C₃₋₆cycloalkyl, and C₂₋₉heterocycloalkyl; wherein C₁₋₆alkyl, C₁₋₆haloalkyl, C₃₋₆ cycloalkyl, and C₂₋₉heterocycloalkyl are optionally substituted with one, two, or three groups selected from R^{9c}. In some embodiments, each R^{6a} is independently selected from halogen. In some embodiments, each R^{6a} is independently selected from C₁₋₆alkyl optionally substituted with one, two, or three groups selected from R^{9c}. In some embodiment, each R^{6a} is independently C₁₋₆haloalkyl optionally substituted with one, two, or three groups selected from R^{9c}. In some embodiments, each R^{6a} is C₃₋₆cycloalkyl optionally substituted with one, two, or three groups selected from R^{9c}. In some embodiment, each R^{6a} is C₂₋₉heterocycloalkyl optionally substituted with one, two, or three groups selected from R^{9c}.

In some embodiments disclosed herein is a compound of Formula (Ia), or a pharmaceutically acceptable salt thereof:

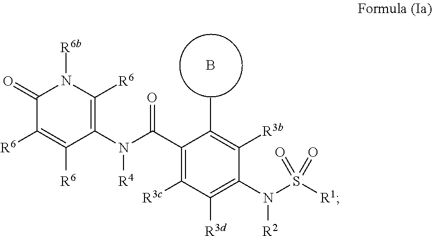

wherein:
each R⁶ and R^{6b} are independently hydrogen or independently selected from R^{6a}; and
each R¹, R², ring B, R^{3b}, R^{3c}, R^{3d}, R⁴ and R^{6a} have the same meanings as described herein.

In some embodiments disclosed herein is a compound of Formula (Iaa), or a pharmaceutically acceptable salt thereof:

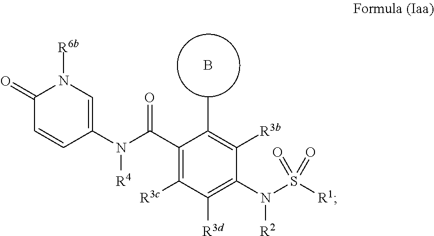

wherein each R¹, R², ring B, R^{3b}, R^{3c}, R^{3d}, R⁴ and R^{6b} have the same meanings as described herein.

In some embodiments disclosed herein is a compound of Formula (Ib), or a pharmaceutically acceptable salt thereof:

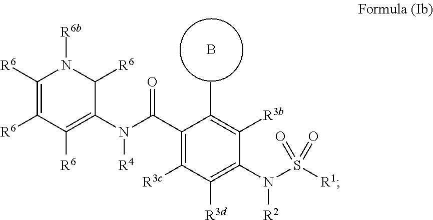

wherein each R¹, R², ring B, R^{3b}, R^{3c}, R^{3d}, R⁴ and R^{6b} have the same meanings as described herein.

In some embodiments disclosed herein is a compound of Formula (Ibb), or a pharmaceutically acceptable salt thereof:

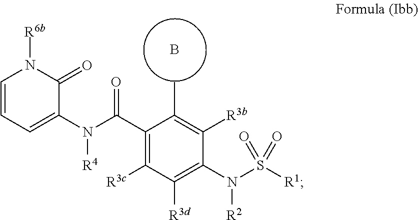

wherein each R¹, R², ring B, R^{3b}, R^{3c}, R^{3d}, R⁴ and R^{6b} have the same meanings as described herein.

In some embodiments disclosed herein is a compound of Formula (Ic), or a pharmaceutically acceptable salt thereof:

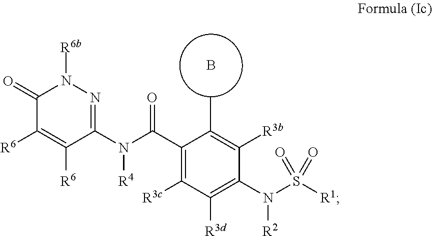

wherein each R¹, R², ring B, R^{3b}, R^{3c}, R^{3d}, R⁴, R⁶ and R^{6b} have the same meanings as described herein.

In some embodiments disclosed herein is a compound of Formula (Icc), or a pharmaceutically acceptable salt thereof:

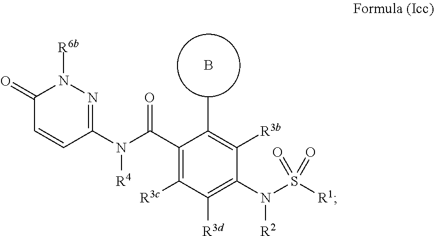

wherein each R¹, R², ring B, R^{3b}, R^{3c}, R^{3d}, R⁴ and R^{6b} have the same meanings as described herein.

In some embodiments of a compound of Formula (Ia), (Iaa), (Ib), (Ibb), (Ic) or (Icc), or a pharmaceutically acceptable salt thereof, R^{6b} is hydrogen. In some embodiments, R^{6b} is R^{6a}. In some embodiments, R^{6b} is R^{6a}, and R^{6a} is selected from C₁₋₆alkyl, C₁₋₆haloalkyl, C₃₋₆cycloalkyl, and C₂₋₉heterocycloalkyl; wherein C₁₋₆alkyl, C₁₋₆haloalkyl, C₃₋₆cycloalkyl, and C₂₋₉heterocycloalkyl are optionally substituted with one, two, or three groups selected from R^{9c}. In some embodiments, or a pharmaceutically acceptable salt thereof, R^{6b} is R^{6a}, and R^{6a} is C₁₋₆alkyl optionally substituted with one, two, or three groups selected from R^{9c}. In some embodiments, R^{6b} is R^{6a}, and R^{6a} is C₁₋₆haloalkyl optionally substituted with one, two, or three groups selected from R^{9c}. In some embodiments, R^{6b} is R^{6a}, and R^{6a} is C₃₋₆cycloalkyl optionally substituted with one, two, or three groups selected from R^{9c}. In some embodiments, R^{6b} is R^{6a}, and R^{6a} is C₂₋₉heterocycloalkyl optionally substituted with one, two, or three groups selected from R⁹⁴. In some embodiments, R^{6b} is R^{6a}, and R^{6a} is 5-6 membered heterocycloalkyl optionally substituted with one, two, or three groups selected from R⁹⁰. In some embodiments, R^{6b} is R^{6a}, and R^{6a} is piperidinyl optionally substituted with one, two, or three groups selected from R^{9c}. In some embodiments, R^{6b} is R^{6a}, and R^{6a} is morpholinyl optionally substituted with one, two, or three groups selected from R^{9c}. In some embodiments, R^{6b} is R^{6a}, and R^{6a} is tetrahydropyranyl optionally substituted with one, two, or three groups selected from R^{9c}.

In some embodiments disclosed herein is a compound of Formula (Id), or a pharmaceutically acceptable salt thereof:

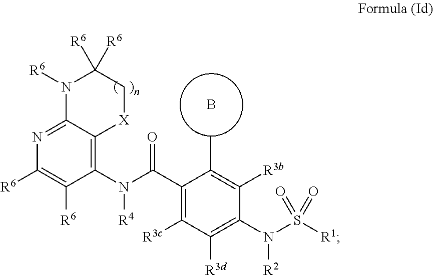

wherein:
X is selected from —O—, —S—, —S(O)—, —S(O)₂—, —C(R^{8a})(R^{8b})—, and —N(R^{8c})—;
R^{8a} and R^{8b} are independently selected from hydrogen, halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9e};
R^{8c} is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9e};
R^{9e} is selected from halogen, oxo, —CN, C₁₋₆alkyl, C₂₋₆alkenyl, C₂₋₆alkynyl, C₃₋₁₀cycloalkyl, —CH₂—C₃₋₆ cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂;
each R¹⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₉alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
n is 1, 2, or 3; and
each R¹, R², ring B, R^{3b}, R^{3c}, R^{3d}, R⁴ and R⁶ have the same meanings as described herein.

In some embodiments disclosed herein is a compound of Formula (Idd), or a pharmaceutically acceptable salt thereof:

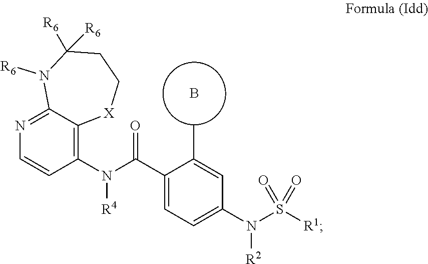

wherein each X, R¹, R², ring B, R⁴ and R⁶ have the same meanings as described herein.

In some embodiments disclosed herein is a compound of Formula (Ie), or a pharmaceutically acceptable salt thereof:

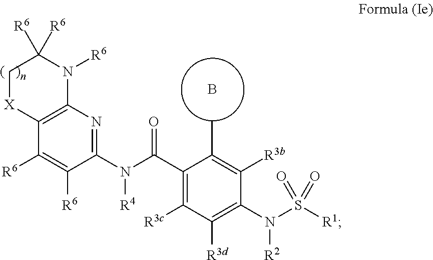

wherein each X, R¹, R², ring B, R⁴ and R⁶ have the same meanings as described herein.

In some embodiments of a compound of Formula (Id) or (Ie), or a pharmaceutically acceptable salt thereof, n is 1. In some embodiments, n is 2. In some embodiments, n is 3.

In some embodiments disclosed herein is a compound of Formula (Iee), or a pharmaceutically acceptable salt thereof:

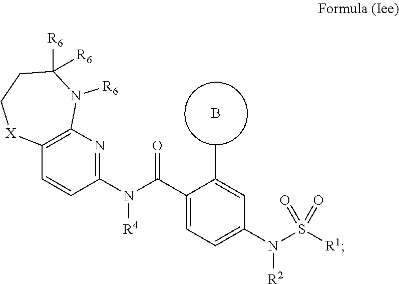

wherein each X, R¹, R², ring B, R⁴ and R⁶ have the same meanings as described herein.

In some embodiments of a compound of Formula (Id), (Idd), (Ie) or (Iee), or a pharmaceutically acceptable salt thereof, X is —O—. In some embodiments, X is —S—. In some embodiments, X is —S(O)—. In some embodiment, X is —S(O)₂—. In some embodiment, X is —C(R^{8a})(R^{8b})—. In some embodiment, X is —CH₂—. In some embodiment, X is —N(R^{8c})—. In some embodiments, X is —N(H)—.

In some embodiments of a compound of Formula (I), (Ia), (Iaa), (Ib), (Ibb), (Ic), (Icc), (Id), (Idd), (Ie) or (Iee), or a pharmaceutically acceptable salt thereof, each R⁶ is independently selected from hydrogen and R^{6a}. In some embodiments, R⁶ is R^{6a}. In some embodiments, one or more R⁶ is R^{6a}. In some embodiments, one or more R⁶ is hydrogen. In some embodiments, each R⁶ is independently selected from hydrogen and R^{6a}, wherein each R^{6a} is independently selected from halogen, C₁₋₆alkyl, C₁₋₆haloalkyl, C₃₋₆ cycloalkyl, and C₂₋₉heterocycloalkyl; wherein C₁₋₆alkyl, C₁₋₆haloalkyl, C₃₋₆cycloalkyl, and C₂₋₉heterocycloalkyl are optionally substituted with one, two, or three groups selected from R^{9e}. In some embodiments, each R⁶ is independently selected from hydrogen and R^{6a}, wherein each R^{6a} is independently selected from halogen and C₁₋₆alkyl optionally substituted with one, two, or three groups selected from R^{9e}. In some embodiments, each R⁶ is independently selected from hydrogen and R^{6a}, wherein each R^{6a} is independently selected from halogen. In some embodiments, each R⁶ is independently selected from hydrogen and R^{6a}, wherein each R^{6a} is independently selected from C₁₋₆alkyl optionally substituted with one, two, or three groups selected from R^{9e}. In some embodiments, each R⁶ is independently selected from hydrogen and R^{6a}, wherein each R^{6a} is independently C₁₋₆haloalkyl optionally substituted with one, two, or three groups selected from R^{9e}. In some embodiments, each R⁶ is hydrogen.

In some embodiments disclosed herein is a compound of Formula (If), or a pharmaceutically acceptable salt thereof:

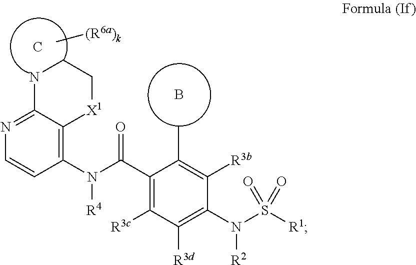

wherein:
ring C is 5-7 membered heterocycloalkyl;
X¹ is selected from —O—, —S—, —S(O)—, —S(O)₂—, —C(R^{8a})(R^{8b})—, and —N(R^{8c})—;
each R^{8a} and each R^{8b} are independently selected from hydrogen, halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9e}; and
each R^{8c} is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9e};
k is 0, 1, 2, 3, 4, 5, or 6; and
each R¹, R², ring B, R^{3b}, R^{3c}, R^{3d}, R^{9e}, R⁴ and R^{6a} have the same meanings as described herein.

In some embodiments disclosed herein is a compound of Formula (Iff), or a pharmaceutically acceptable salt thereof:

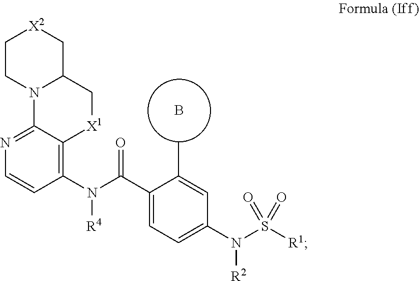

wherein:
X¹ and X² are each independently selected from —O—, —S—, —S(O)—, —S(O)₂—, —C(R^{8a})(R^{8b})—, and —N(R^{8c})—; each R^{8a} and each R^{8b} are independently selected from hydrogen, halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₁₋₆haloalkyl, C₂₋₆alkenyl, C₂₋₉alkynyl, C₃₋₆cycloalkyl, C₂₋₉heterocycloalkyl, C₆₋₁₀aryl, and C₁₋₉heteroaryl are optionally substituted with one, two, or three groups selected from R⁹;
each R^{8c} is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9e};
each R¹, R², R^{9e}, ring B and R⁴ have the same meanings as described herein.

In some embodiments disclosed herein is a compound of Formula (Ig), or a pharmaceutically acceptable salt thereof:

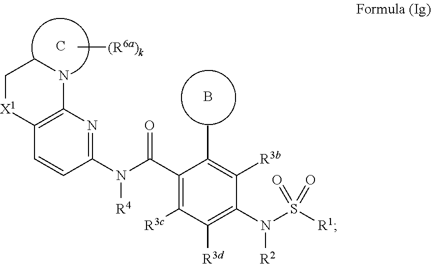

wherein each X¹, k, R¹, R², ring B, ring C, R^{3b}, R^{3c}, R^{3d}, R⁴ and R^{6a} have the same meanings as described herein.

In some embodiments of a compound of Formula (If) or (Ig), or a pharmaceutically acceptable salt thereof, ring C is 5-membered heterocycloalkyl. In some embodiments, ring C is 6-membered heterocycloalkyl. In some embodiments, ring C is 7-membered heterocycloalkyl.

In some embodiments of a compound of Formula (I), (Ia), (Iaa), (Ib), (Ibb), (Ic), (Icc), (Id), (Ie), (If) or (Ig), or a pharmaceutically acceptable salt thereof, R^{3b} is selected from hydrogen, halogen, —CN, and C₁₋₆alkyl optionally substituted with one, two, or three groups selected from R^{9c}. In some embodiments, R^{3b} is hydrogen. In some embodiments, R^{3b} is halogen. In some embodiments, R^{3b} is C₁₋₆alkyl optionally substituted with one, two, or three groups selected from R^{9c}.

In some embodiments of a compound of Formula (I), (Ia), (Iaa), (Ib), (Ibb), (Ic), (Icc), (Id), (Ie), (If) or (Ig), or a pharmaceutically acceptable salt thereof, R^{3b} is selected from hydrogen, halogen, —CN, and C₁₋₆alkyl optionally substituted with one, two, or three groups selected from R^{9c}. In some embodiments, R^{3b} is hydrogen. In some embodiments, R^{3b} is halogen. In some embodiments, R^{3c} is C₁₋₆alkyl optionally substituted with one, two, or three groups selected from R^{9c}.

In some embodiments of a compound of Formula (I), (Ia), (Iaa), (Ib), (Ibb), (Ic), (Icc), (Id), (Ie), (If) or (Ig), or a pharmaceutically acceptable salt thereof, R^{3d} is selected from hydrogen, halogen, —CN, and C₁₋₆alkyl optionally substituted with one, two, or three groups selected from R^{9d}. In some embodiments, R^{3d} is hydrogen. In some embodiments, R^{3d} is halogen. In some embodiments, R^{3d} is C₁₋₆alkyl optionally substituted with one, two, or three groups selected from R^{9d}.

In some embodiments of a compound of Formula (I), (Ia), (Iaa), (Ib), (Ibb), (Ic), (Icc), (Id), (Ie), (If) or (Ig), or a pharmaceutically acceptable salt thereof, R² and R^{3d} are combined to form a C₂₋₉ heterocycloalkyl or C₂₋₉heteroaryl, wherein the C₂₋₉heterocycloalkyl and C₂₋₉heteroaryl are optionally substituted with one, two, or three groups selected from R^{9d}. In some embodiments, R² and R^{3d} are combined to form an optionally substituted 5-6 membered heterocycloalkyl. In some embodiments, R² and R^{3d} are combined to form an optionally substituted 5-6 membered heteroaryl. In some embodiments, R² and R^{3d} are combined to form a C₂₋₉heterocycloalkyl optionally substituted with one, two, or three groups selected from R^{9d}. In some embodiments, R² and R^{3d} are combined to form a pyrrolidine ring optionally substituted with one, two, or three groups selected from R^{9d}. In some embodiments, R² and R^{3d} are combined to form a C₂₋₉heteroaryl optionally substituted with one, two, or three groups selected from R^{9d}. In some embodiments, R² and R^{3d} are combined to form a pyrazole ring optionally substituted with one, two, or three groups selected from R^{9d}. In some embodiments, or a pharmaceutically acceptable salt thereof, R² and R^{3d} are combined to form a pyrrole ring optionally substituted with one, two, or three groups selected from R^{9d}.

In some embodiments of a compound of Formula (I), (Ia), (Iaa), (Ib), (Ibb), (Ic), (Icc), (Id), (Idd), (Ie), (Tee), (If) or (Ig), or a pharmaceutically acceptable salt thereof, each R^{6a} is independently selected from halogen, C₁₋₆alkyl, C₁₋₆haloalkyl, C₃₋₆cycloalkyl, and C₂₋₉heterocycloalkyl; wherein C₁₋₆alkyl, C₁₋₆haloalkyl, C₃₋₆cycloalkyl, and C₂₋₉heterocycloalkyl are optionally substituted with one, two, or three groups selected from R^{9e}. In some embodiments, each R^{6a} is independently selected from halogen and C₁₋₆alkyl optionally substituted with one, two, or three groups selected from R^{9e}. In some embodiments, each R^{6a} is independently selected from halogen. In some embodiments, each R^{6a} is independently selected from C₁₋₆alkyl optionally substituted with one, two, or three groups selected from R^{9e}. In some embodiments, R^{6a}, wherein each R^{6a} is independently C₁₋₆haloalkyl optionally substituted with one, two, or three groups selected from R^{9e}.

In some embodiments of a compound of Formula (If) or (Ig), or a pharmaceutically acceptable salt thereof, k is 0. In some embodiments, k is 1. In some embodiments, k is 2. In some embodiments, k is 3.

In some embodiments disclosed herein is a compound of Formula (Igg), or a pharmaceutically acceptable salt thereof:

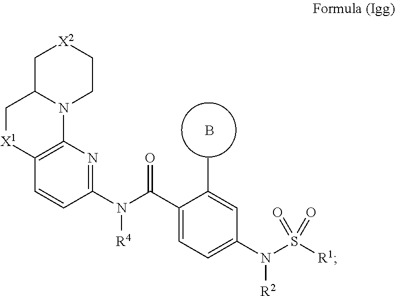

wherein:
X¹ and X² are independently selected from —O—, —S—, —S(O)—, —S(O)₂—, —C(R^{8a})(R^{8b})—, and —N(R^{8c})—;
each R¹, R², R^{8a}, R^{8b}, R^{8c}, ring B and R⁴ have the same meanings as described herein.

In some embodiments of a compound of Formula (Iff) or (Igg), or a pharmaceutically acceptable salt thereof, X² is —O—. In some embodiments, X² is —S—. In some embodiments, X² is —S(O)—. In some embodiments, X² is —S(O)₂—. In some embodiments, X² is —C(R^{8a})(R^{8b})—. In some embodiments, X² is —CH₂—. In some embodiments, X² is —N(R¹¹)—. In some embodiments, X² is —N(H)—.

In some embodiments of a compound of Formula (I), (Ia), (Iaa), (Ib), (Ibb), (Ic), (Icc), (Id), (Idd), (Ie), (Iee), (If), (Ig), (Iff) or (Igg), or a pharmaceutically acceptable salt thereof, R² is hydrogen. In some embodiments, R² is C₁₋₆alkyl.

In some embodiments of a compound of Formula (If), (Iff), (Ig) or (Igg), or a pharmaceutically acceptable salt thereof, X¹ is —O—. In some embodiments, X¹ is —S—. In some embodiments, X¹ is —S(O)—. In some embodiments, X¹ is —S(O)₂—. In some embodiments, X¹ is —C(R¹¹)(R^{8b})—. In some embodiments, X¹ is —CH₂—. In some embodiments, X¹ is —N(R¹¹)—. In some embodiments, X¹ is —N(H)—.

In some embodiments of a compound of Formula (I), (Ia), (Iaa), (Ib), (Ibb), (Ic), (Icc), (Id), (Idd), (Ie), (Iee), (If), (Iff), (Ig) or (Igg), or a pharmaceutically acceptable salt thereof, ring B is a monocyclic ring. In some embodiments, ring B is a bicyclic ring. In some embodiments, ring B is C₂₋₁₄heterocycloalkyl optionally substituted with one, two, or three groups selected from R^{9b}. In some embodiments, ring B is an unsubstituted C₂₋₁₄heterocycloalkyl. In some embodiments, ring B is

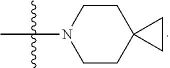 [SMILES: *N1CCC2(CC1)CC2]

In some embodiments of a compound of Formula (I), (Ia), (Iaa), (Ib), (Ibb), (Ic), (Icc), (Id), (Idd), (Ie), (Tee), (If), (Iff), (Ig) or (Igg), or a pharmaceutically acceptable salt thereof, ring B is C₃₋₁₂ cycloalkyl optionally substituted with one, two, or three groups selected from R^{9b}. In some embodiments, ring B is C₃₋₁₂cycloalkyl optionally substituted with one, two, or three groups selected from R^{9b}, wherein each R^{9b} is halogen. In some embodiments, ring B is an unsubstituted C₃₋₁₂cycloalkyl. In some embodiments, ring B is selected from

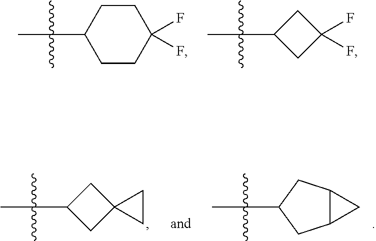 [SMILES: *C1CCC(F)(F)CC1 ; *C1CC(F)(F)C1 ; *C1CC2(CC2)C1 ; *C1CC2CC2C1]

In some embodiments, ring B is

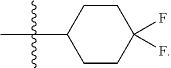 [SMILES: *C1CCC(F)(F)CC1]

In some embodiments, ring B is

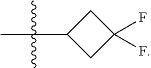 [SMILES: *C1CC(F)(F)C1]

In some embodiments, ring B is

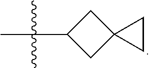 [SMILES: *C1CC2(CC2)C1]

In some embodiments, ring B is

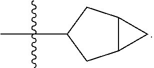 [SMILES: *C1CC2CC2C1]

In some embodiments of a compound of Formula (I), (Ia), (Iaa), (Ib), (Ibb), (Ic), (Icc), (Id), (Idd), (Ie), (Iee), (If), (Iff), (Ig) or (Igg), or a pharmaceutically acceptable salt thereof, ring B is C₁₋₉heteroaryl optionally substituted with one, two, or three groups selected from R^{9b}.

In some embodiments of a compound of Formula (I), (Ia), (Iaa), (Ib), (Ibb), (Ic), (Icc), (Id), (Idd), (Ie), (Iee), (If), (Iff), (Ig) or (Igg), or a pharmaceutically acceptable salt thereof, ring B is C₆₋₁₀aryl optionally substituted with one, two, or three groups selected from R^{9b}. In some embodiments, ring B is phenyl optionally substituted with one, two, or three groups selected from R^{9b}.

In some embodiments of a compound of Formula (I), (Ia), (Iaa), (Ib), (Ibb), (Ic), (Icc), (Id), (Idd), (Ie), (Tee), (If), (Iff), (Ig) or (Igg), or a pharmaceutically acceptable salt thereof, R¹ is C₁₋₆alkyl optionally substituted with one, two, or three groups selected from R^{a}. In some embodiments, R¹ is C₁₋₆alkyl optionally substituted with one, two, or three groups substituents selected from —OH and C₁₋₆alkoxy. In some embodiments, R¹ is C₁₋₆alkylene substituted with one —OH group. In some embodiments, R¹ is —CH₂CH₂OH or —CH₂CH₂CH₂OH. In some embodiments, R¹ is —CH₂CH₂OH. In some embodiments, R¹ is —CH₂CH₂CH₂OH.

In some embodiments of a compound of Formula (I), (Ia), (Iaa), (Ib), (Ibb), (Ic), (Icc), (Id), (Idd), (Ie), (Iee), (If), (Iff), (Ig) or (Igg), or a pharmaceutically acceptable salt thereof, R¹ is C₃₋₆cycloalkyl optionally substituted with one, two, or three groups selected from R^{9a}.

In some embodiments of a compound of Formula (I), (Ia), (Iaa), (Ib), (Ibb), (Ic), (Icc), (Id), (Idd), (Ie), (Tee), (If), (Iff), (Ig) or (Igg), or a pharmaceutically acceptable salt thereof, R⁴ is hydrogen. In some embodiments, R⁴ is C₁₋₆alkyl.

In some embodiments of a compound of Formula (I), (Ia), (Iaa), (Ib), (Ibb), (Ic), (Icc), (Id), (Idd), (Ie), (Tee), (If), (Iff), (Ig) or (Igg), or a pharmaceutically acceptable salt thereof, R² is hydrogen. In some embodiments, R² is C₁₋₆alkyl.

In some embodiments disclosed herein is a compound of Formula (II), or a pharmaceutically acceptable salt thereof:

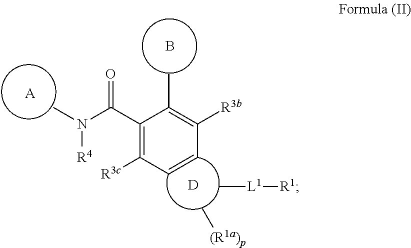

wherein:
ring A is selected from C₂₋₁₄heterocycloalkyl, C₂₋₁₄heteroaryl, and C₆₋₁₀aryl, wherein C₂₋₁₄heterocycloalkyl, C₂₋₁₄heteroaryl, and C₆₋₁₀aryl are optionally substituted with one or more R^{6a} groups;
ring D is selected from a 5- to 6-membered heterocycloalkyl ring, a 5- to 6-membered heteroaryl ring, and a 5- to 6-membered cycloalkyl ring;
L¹ is selected from —S(O)₂— and —C(O)—;
L² is selected from a bond, —O—, —S—, —S(O)—, —S(O)₂—, —C(R^{8a})(R^{8b})—, —OC(R^{8a})(R^{8b})—, —C(R^{8a})(R^{8b})O—, and —N(R^{8c})—;
R¹ is selected from C₁₋₆alkyl and C₃₋₆cycloalkyl, wherein C₁₋₆alkyl and C₃₋₆cycloalkyl are optionally substituted with one, two, or three groups selected from R^{9a};
each R^{1a} is independently selected from halogen, —CN, oxo, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(=NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₆heteroaryl are optionally substituted with one, two, or three groups selected from R^{9d};
ring B is selected from C₃₋₁₂cycloalkyl, C₂₋₁₄heterocycloalkyl, C₆₋₁₀aryl, and C₁₋₉heteroaryl, wherein C₃₋₁₂cycloalkyl, C₂₋₁₄heterocycloalkyl, C₆₋₁₀aryl, and C₁₋₉heteroaryl are optionally substituted with one, two, or three groups selected from R^{9b};
R^{3b} and R^{3c} are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9c};
R⁴ is selected from hydrogen and C₁₋₆alkyl;
each R^{6a} is independently selected from halogen, hydroxy, oxo, —CN, C₁₋₆alkyl, C₁₋₆haloalkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹⁰, —C(O)R¹³, —C(O)N(R¹⁰)(R¹¹), —S(O)₂R¹³, and —S(O)₂N(R¹⁰)(R¹¹)—; wherein C₁₋₆alkyl, C₁₋₆haloalkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9e};
R^{8a} and R^{8b} are independently selected from hydrogen, halogen, C₁₋₁₀alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three groups selected from R^{9f};
R^{8c} is selected from hydrogen, C₁₋₆alkyl, C₁₋₆haloalkyl, C₂₋₆alkenyl, C₂₋₆alkynyl, C₃₋₁₀cycloalkyl, C₂₋₉heterocycloalkyl, C₁₋₁₀aryl, and C₁₋₉heteroaryl; wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9f};
each R^{9a}, R^{9b}, R^{9c}, R^{9d}, R^{9e}, and R^{9f} are each independently selected from halogen, oxo, —CN, C₁₋₆ alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂;
each R¹⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
p is 0, 1, 2, or 3.

In some embodiments of a compound of Formula (II), or a pharmaceutically acceptable salt thereof, ring D is a 5- to 6-membered heterocycloalkyl ring. In some embodiments, ring D is a 5- to 6-membered heteroaryl ring. In some embodiments, ring D is a 5- to 6-membered cycloalkyl ring.

In some embodiments of a compound of Formula (II), or a pharmaceutically acceptable salt thereof, ring A is a monocyclic ring. In some embodiments, ring A is a bicyclic ring. In some embodiments, ring A is a tricyclic ring. In some embodiments, ring A is C₂₋₁₄heterocycloalkyl optionally substituted with one or more R^{6a} groups. In some embodiments, ring A is C₂₋₁₄heteroaryl optionally substituted with one or more R^{6a} groups. In some embodiments, ring A is optionally substituted 6 membered heteroaryl. In some embodiments, ring A is C₆₋₁₀aryl optionally substituted with one or more R^{6a} groups. In some embodiments, ring A is a phenyl, a pyrimidinyl, a pyridyl, a pyridazinonyl, an 11-15 membered bicyclic heterocyclic ring or an 11-17 membered tricyclic heterocyclic ring, wherein the phenyl, pyrimidinyl, pyridyl, pyridazinonyl, 11-15 membered bicyclic heterocyclic ring and 11-17 membered tricyclic heterocyclic ring are optionally substituted with one or more R^{6a} groups. In some embodiments, ring A is a phenyl are optionally substituted with one or more R^{6a} groups. In some embodiments, ring A is a pyrimidinyl optionally substituted with one or more R^{6a} groups. In some embodiments, ring A is a pyridyl optionally substituted with one or more R^{6a} groups. In some embodiments, ring A is a pyridazinonyl optionally substituted with one or more R^{6a} groups. In some embodiments, ring A is an 11-15 membered bicyclic heterocyclic ring optionally substituted with one or more R^{6a} groups. In some embodiments, ring A is an 11-17 membered tricyclic heterocyclic ring optionally substituted with one or more R^{6a} groups.

In some embodiments of a compound of Formula (II), or a pharmaceutically acceptable salt thereof, each R^{6a} is independently selected from halogen, C₁₋₆alkyl, C₁₋₆haloalkyl, C₃₋₆cycloalkyl, C₂₋₉heterocycloalkyl, —S(O)₂R¹³, and —S(O)₂N(R¹⁰)(R¹¹)—; wherein C₁₋₆alkyl, C₁₋₆haloalkyl, C₃₋₆cycloalkyl, and C₂₋₉heterocycloalkyl are optionally substituted with one, two, or three groups selected from R^{9e}. In some embodiments, each R^{6a} is independently selected from halogen, C₁₋₆alkyl, C₁₋₆haloalkyl, C₃₋₆cycloalkyl, and C₂₋₉heterocycloalkyl; wherein C₁₋₆alkyl, C₁₋₆haloalkyl, C₃₋₆cycloalkyl, and C₂₋₉heterocycloalkyl are optionally substituted with one, two, or three groups selected from R^{9e}. In some embodiments, each R^{6a} is independently selected from halogen. In some embodiments, each R^{6a} is independently selected from C₁₋₆alkyl optionally substituted with one, two, or three groups selected from R^{9e}. In some embodiments, each R^{6a} is independently C₁₋₆haloalkyl optionally substituted with one, two, or three groups selected from R^{9e}. In some embodiments, or a pharmaceutically acceptable salt thereof, each R^{6a} is C₃₋₆cycloalkyl optionally substituted with one, two, or three groups selected from R^{9e}. In some embodiments, each R^{6a} is C₂₋₉heterocycloalkyl optionally substituted with one, two, or three groups selected from R^{9e}. In some embodiments of a compound of Formula (IIa), (IIaa), (IIb), (IIbb), (IIbb′), (IIcc) or (IIc), R⁶ is R^{6a}. In some embodiments of a compound of Formula (IIa), (IIaa), (IIb), (IIbb), (IIbb′), (IIcc) or (IIc), R^{6b} is R^{6a}.

In some embodiments of a compound of Formula (II), or a pharmaceutically acceptable salt thereof, each R^{1a} is independently selected from halogen and C₁₋₆alkyl optionally substituted with one, two, or three groups selected from R^{9d}.

In some embodiments of a compound of Formula (II), or a pharmaceutically acceptable salt thereof, p is 2. In some embodiments, p is 1. In some embodiments, p is 0. In some embodiments disclosed herein is a compound of Formula (IIa), or a pharmaceutically acceptable salt thereof:

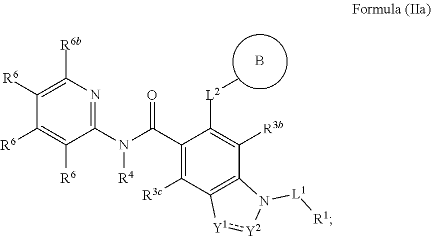

wherein:
[symbol] is a single bond or a double bond;
Y¹ is N, —C(R^{Ya})(R^{Yb})—, or —C(R^{Ya})—;
Y² is N, —C(R^{Ya})(R^{Yb})—, or —C(R^{Ya})—;
each R^{Ya} and each R^{Yb} are independently selected from hydrogen, halogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₁₋₉haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9d}; or R^{Ya} and R^{Yb} on the same carbon are taken together to form an oxo;
L¹ is selected from —S(O)₂— and —C(O)—;
L² is selected from a bond, —O—, —S—, —S(O)—, —S(O)₂—, —C(R^{8a})(R^{8b})—, —OC(R^{8a})(R^{8b})—, —C(R^{8a})(R^{8b})O—, and —N(R^{8c})—;
R¹ is selected from C₁₋₆alkyl and C₃₋₆cycloalkyl, wherein C₁₋₆alkyl and C₃₋₆cycloalkyl are optionally substituted with one, two, or three groups selected from R^{9a};
ring B is selected from C₃₋₁₂cycloalkyl, C₂₋₁₄heterocycloalkyl, C₆₋₁₀aryl, and C₁₋₉heteroaryl, wherein C₃₋₁₂cycloalkyl, C₂₋₁₄heterocycloalkyl, C₆₋₁₀aryl, and C₁₋₆heteroaryl are optionally substituted with one, two, or three groups selected from R^{9b};
R^{3b} and R^{3c} are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹¹, —SR¹⁰, —SF₅, —N(R¹⁰)(R¹¹), —C(O)OR¹⁰, —OC(O)N(R¹⁰)(R¹¹), —N(R²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9c};
R⁴ is selected from hydrogen and C₁₋₆alkyl;
each R⁶ and R^{6b} is independently hydrogen or independently selected from R^{a};
each R^{6a} is independently selected from halogen, hydroxy, oxo, —CN, C₁₋₆alkyl, C₁₋₆haloalkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹⁰, —C(O)R¹³, —C(O)N(R¹⁰)(R¹¹), —S(O)₂R¹³, and —S(O)₂N(R¹⁰)(R¹¹)—; wherein C₁₋₆alkyl, C₁₋₆haloalkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R⁹;
R^{8a} and R^{8b} are independently selected from hydrogen, halogen, C₁₋₆alkyl, C₁₋₉haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₁₀heteroaryl are optionally substituted with one, two, or three groups selected from R^{9f};
R^{8c} is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₁₀heteroaryl are optionally substituted with one, two, or three groups selected from R^{9f};
each R^{9a}, R^{9b}, R^{9c}, R^{9d}, R^{9e}, and R^{9f} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroacyl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂;
each R¹⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₆heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆ alkoxy, C₃₋₆cycloalkyl, C₂₋₉heterocycloalkyl, C₆₋₁₀aryl, and C₁₋₉heteroaryl.

In some embodiments disclosed herein is a compound of Formula (IIaa), or a pharmaceutically acceptable salt thereof:

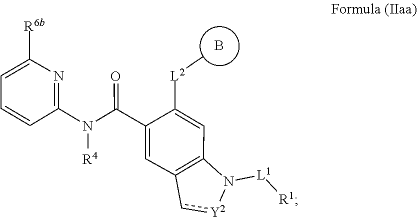

wherein each Y², L¹, L², R¹, R^{6b}, ring B and R⁴ have the same meanings as described herein.

In some embodiments disclosed herein is a compound of Formula (IIb), or a pharmaceutically acceptable salt thereof:

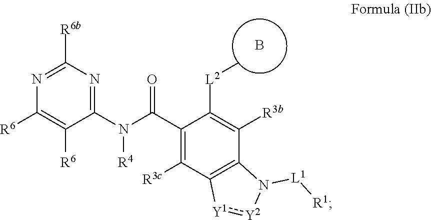

wherein each Y¹, Y², L¹, L², R¹, R⁶, R^{6b}, R^{3b}, R^{3c}, ring B and R⁴ have the same meanings as described herein.

In some embodiments disclosed herein is a compound of Formula (IIbb), or a pharmaceutically acceptable salt thereof:

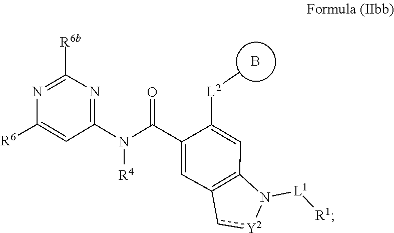

wherein each Y², L¹, L², R¹, R⁶, R^{6b}, ring B and R⁴ have the same meanings as described herein.

In some embodiments disclosed herein is a compound of Formula (IIbb′), or a pharmaceutically acceptable salt thereof:

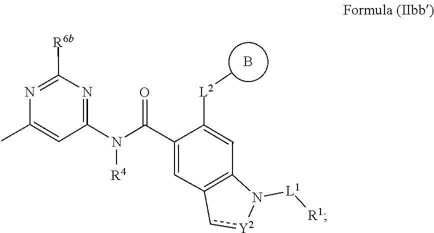

wherein each Y², L¹, L², R¹, R^{bb}, ring B and R⁴ have the same meanings as described herein.

In some embodiments disclosed herein is a compound of Formula (IIc), or a pharmaceutically acceptable salt thereof:

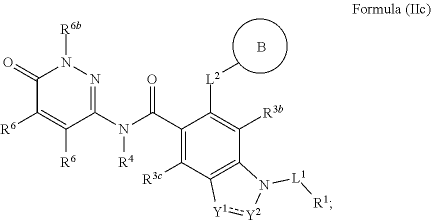

wherein each Y¹, Y², L¹, L², R¹, R⁶, R^{6b}, R^{3b}, R^{3c}, ring B and R⁴ have the same meanings as described herein.

In some embodiments of a compound of Formula (IIa), (IIb) or (IIc), or a pharmaceutically acceptable salt thereof, Y¹ is —C(R^{Ya})(R^{Yb})—. In some embodiments, Y¹ is CH or CH₂. In some embodiments, Y¹ is —C(R^{Ya})—. In some embodiments, Y¹ is CH₂. In some embodiments, Y¹ is —N—. In some embodiments, Y¹ is —NR¹⁰—. In some embodiments, Y¹ is —NH—.

In some embodiments of a compound of Formula (II), (IIa), (IIb) or (IIc), or a pharmaceutically acceptable salt thereof, R^{3b} is selected from hydrogen, halogen, —CN, and C₁₋₆alkyl optionally substituted with one, two, or three groups selected from R^{9c}. In some embodiments, R^{3b} is hydrogen. In some embodiments, R^{3b} is halogen. In some embodiments, R^{3b} is C₁₋₆alkyl optionally substituted with one, two, or three groups selected from R^{9c}.

In some embodiments of a compound of Formula (II), (IIa), (IIb) or (IIc), or a pharmaceutically acceptable salt thereof, R^{3c} is selected from hydrogen, halogen, —CN, and C₁₋₆alkyl optionally substituted with one, two, or three groups selected from R^{9c}. In some embodiments, R^{3e} is hydrogen. In some embodiment, R^{3c} is halogen. In some embodiments, R^{3c} is C₁₋₆alkyl optionally substituted with one, two, or three groups selected from R^{9c}.

In some embodiments of a compound of Formula (IIa), (IIb), (IIbb), or (Tic), or a pharmaceutically acceptable salt thereof, each R⁶ is independently selected from hydrogen and R^{6a}. In some embodiments of a compound of Formula (IIa), (IIaa), (IIb), (IIbb), (IIbb′), (IIcc) or (IIc), or a pharmaceutically acceptable salt thereof, each R^{6b} is independently selected from hydrogen and R^{6a}. In some embodiments, R⁶ is hydrogen. In some embodiments, R⁶ is R^{6b}. In some embodiments, one or more R⁶ is hydrogen. In some embodiments, one or more R⁶ is R^{6b}. In some embodiments, R^{6b} is hydrogen. In some embodiments, R^{6b} is R^{6a}. In some embodiments, each R⁶ and R^{6b} is independently selected from hydrogen and R^{6a}, wherein each R^{6a} is independently selected from halogen, C₁₋₆alkyl, C₁₋₆haloalkyl, C₃₋₆ cycloalkyl, and C₂₋₉heterocycloalkyl; wherein C₁₋₆alkyl, C₁₋₆haloalkyl, C₃₋₆cycloalkyl, and C₂₋₉heterocycloalkyl are optionally substituted with one, two, or three groups selected from R^{9c}. In some embodiments, each R⁶ and R^{6b} is independently selected from hydrogen and R^{6a}, wherein each R^{6a} is independently selected from halogen. In some embodiments, each R⁶ and R^{6b} is independently selected from hydrogen and R^{6a}, wherein each R^{6a} is independently selected from C₁₋₆alkyl optionally substituted with one, two, or three groups selected from R^{9c}. In some embodiments, each R⁶ and R^{6b} is independently selected from hydrogen and R^{6a}, wherein each R^{6a} is independently C₁₋₆haloalkyl optionally substituted with one, two, or three groups selected from R^{9c}. In some embodiments, each R⁶ and R^{6b} is independently selected from hydrogen and R^{6a}, wherein each R^{6a} is C₃₋₆cycloalkyl optionally substituted with one, two, or three groups selected from R^{9e}. In some embodiments, each R⁶ and R^{6b} is independently selected from hydrogen and R^{6a}, wherein each R^{6a} is C₂₋₉heterocycloalkyl optionally substituted with one, two, or three groups selected from R^{9e}. In some embodiments, each R⁶ and R^{6b} is independently selected from hydrogen and R^{6a}, wherein each R^{6a} is selected from —CH₂CH₂CF₃, —CH₂CH₂CH₂CF₃,

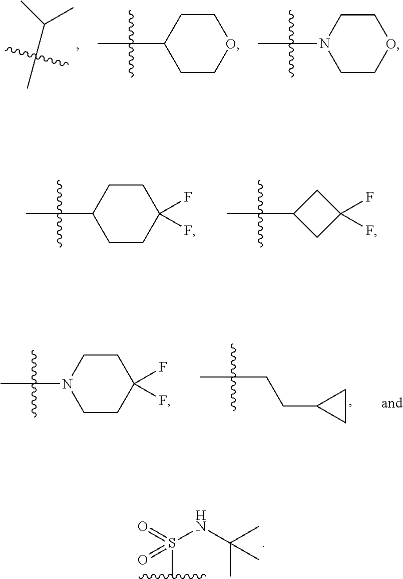

In some embodiments, each R⁶ is hydrogen and R^{6b} is R^{6a}, wherein R^{6a} is selected from —CH₂CH₂CF₃, —C₂CH₂CH₂CF₃,

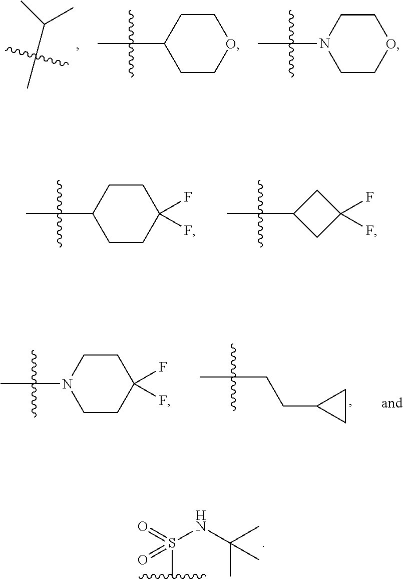

In some embodiments, each R⁶ is hydrogen and R^{6b} is R^{6a}, wherein R^{6a} is —CH₂CH₂CF₃. In some embodiments, each R⁶ is hydrogen and R^{6b} is R^{6a}, wherein R^{6a} is —CH₂CH₂CH₂CF₃.

In some embodiments, each R⁶ is hydrogen and R^{6b} is R^{6a}, wherein R^{6a} is

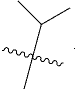 [SMILES: *C(C)C]

In some embodiments, each R⁶ is hydrogen and R^{6b} is R^{6a}, wherein R^{6a} is

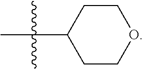 [SMILES: *C1CCOCC1]

In some embodiments, each R⁶ is hydrogen and R^{6b} is R^{6a}, wherein R^{6a} is

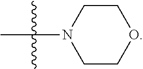 [SMILES: *N1CCOCC1]

In some embodiments, each R⁶ is hydrogen and R^{6b} is R^{6a}, wherein R^{6a} is

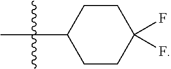 [SMILES: *C1CCC(F)(F)CC1]

In some embodiments, each R⁶ is hydrogen and R^{6b} is R^{6a}, wherein R^{6a} is

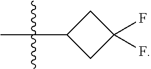 [SMILES: *C1CC(F)(F)C1]

In some embodiments, each R⁶ is hydrogen and R^{6b} is R^{6a}, wherein R^{6a} is

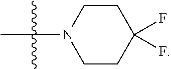 [SMILES: *N1CCC(F)(F)CC1]

In some embodiments, each R⁶ is hydrogen and R^{6b} is R^{6a}, wherein R^{6a} is

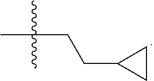 [SMILES: *CCC1CC1]

In some embodiments, each R⁶ is hydrogen and R^{6b} is R^{6a}, wherein R^{6a} is

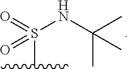 [SMILES: *S(=O)(=O)NC(C)(C)C]

In some embodiments disclosed herein is a compound of Formula (IIcc), or a pharmaceutically acceptable salt thereof:

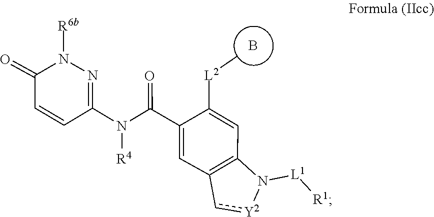

wherein each Y², L¹, L², R¹, R^{6b}, ring B and R⁴ have the same meanings as described herein.

In some embodiments of a compound of Formula (IIa), (IIaa), (IIb), (IIbb), (IIbb′), (IIc) or (IIcc), or a pharmaceutically acceptable salt thereof, R^{6b} is hydrogen. In some embodiments, or a pharmaceutically acceptable salt thereof, R^{6b} is R^{6a}. In some embodiments, R^{6b} is R^{6a}, and R^{6a} is selected from C₁₋₆alkyl, C₁₋₆haloalkyl, C₃₋₆cycloalkyl, and C₂₋₉heterocycloalkyl; wherein C₁₋₆alkyl, C₁₋₆haloalkyl, C₃₋₆cycloalkyl, and C₂₋₉heterocycloalkyl are optionally substituted with one, two, or three groups selected from R^{9e}. In some embodiment, Rib is R^{6a}, and R^{6a} is C₁₋₆alkyl optionally substituted with one, two, or three groups selected from R^{9e}. In some embodiments, Rib is R^{6a}, and R^{6a} is C₁₋₆haloalkyl optionally substituted with one, two, or three groups selected from R^{9e}. In some embodiments, R^{6b} is R^{6a}, and R^{6a} is C₃₋₆cycloalkyl optionally substituted with one, two, or three groups selected from R^{9e}. In some embodiments, R^{6b} is R^{6a}, and R^{6a} is C₂₋₉heterocycloalkyl optionally substituted with one, two, or three groups selected from R^{9e}. In some embodiments, R^{6b} is R^{6a}, and R^{6a} is piperidinyl optionally substituted with one, two, or three groups selected from R^{9e}. In some embodiments, R^{6b} is R^{6a}, and R^{6a} is morpholinyl optionally substituted with one, two, or three groups selected from R^{9e}. In some embodiments, R^{6b} is R^{6a}, and R^{6a} is tetrahydropyranyl optionally substituted with one, two, or three groups selected from R^{9e}. In some embodiments, R^{6b} is R^{6a}, wherein R^{6a} is selected from —CH₂CH₂CF₃, —CH₂CH₂CH₂CF₃,

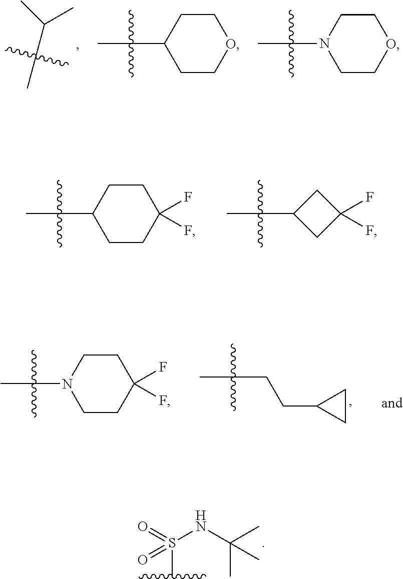

In some embodiments, R^{6b} is R^{6a}, wherein R^{6a} is —CH₂CH₂CF₃. In some embodiments, R^{6b} is R^{6A}, wherein R^{6A} is —CH₂CH₂CH₂CF₃. In some embodiments, R^{6b} is R^{6A}, wherein R^{6a} is

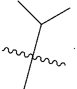 [SMILES: *C(C)C]

In some embodiments, R^{6b} is R^{6a}, wherein R^{6a} is

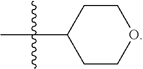 [SMILES: *C1CCOCC1]

In some embodiments, R^{6b} is R^{6a}, wherein R^{6a} is

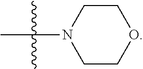 [SMILES: *N1CCOCC1]

In some embodiments, R^{6b} is R^{6a}, wherein R^{6a} is

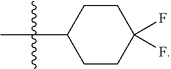 [SMILES: *C1CCC(F)(F)CC1]

In some embodiments, R^{6b} is R^{6a}, wherein R^{6a} is

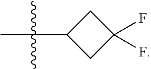 [SMILES: *C1CC(F)(F)C1]

In some embodiments, R^{6b} is R^{6a}, wherein R^{6a} is

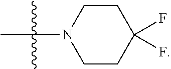 [SMILES: *N1CCC(F)(F)CC1]

In some embodiments, R^{6b} is R^{6a}, wherein R^{6a} is

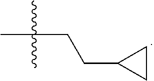 [SMILES: *CCC1CC1]

In some embodiments, R^{6b} is R^{6a}, wherein R^{6a} is

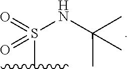 [SMILES: *S(=O)(=O)NC(C)(C)C]

In some embodiments of a compound of Formula (IIa (IIaa), (IIb), (IIbb), (IIbb′), (IIc) or (IIcc), or a pharmaceutically acceptable salt thereof, [symbol] is a single bond. In some embodiments, [symbol] is a double bond.

In some embodiments of a compound of Formula (IIa), (IIaa), (IIb), (IIbb), (IIbb′), (IIc) or (IIcc), or a pharmaceutically acceptable salt thereof, Y² is —C(R^{Ya})(R^{Yb})—. In some embodiments, Y² is CH or CH₂. In some embodiments, Y² is —C(R^{Ya})—. In some embodiments, Y² is CH₂. In some embodiments, Y² is —N—.

In some embodiments of a compound of Formula (II), (IIa), (IIaa), (IIb), (IIbb), (IIbb′), (IIc) or (IIcc), or a pharmaceutically acceptable salt thereof, L¹ is —S(O)₂—. In some embodiments, L¹ is —C(O)—.

In some embodiments of a compound of Formula (II), (IIa), (IIaa), (IIb), (IIbb), (IIbb′), (IIc) or (IIcc), or a pharmaceutically acceptable salt thereof, L² is —O—. In some embodiments, L² is —S—. In some embodiments, L² is —S(O)—. In some embodiments, L² is —S(O)₂—. In some embodiments, L² is —C(R^{8a})(R^{8b})—. In some embodiments, L² is —CH₂—. In some embodiments, L² is —OC(R^{8a})(R^{8b})—. In some embodiments, L² is —OCH₂—. In some embodiments, L² is —C(R^{8a})(R^{8b})O—. In some embodiments, L² is —CH₂O—. In some embodiments, L² is —N(R^{8c})—. In some embodiments, L² is —N(H)—.

In some embodiments of a compound of Formula (II), (IIa), (IIaa), (IIb), (IIbb), (IIbb′), (IIc) or (IIcc), or a pharmaceutically acceptable salt thereof, ring B is a monocyclic ring. In some embodiments, ring B is a bicyclic ring. In some embodiments, ring B is C₂₋₁₄ heterocycloalkyl optionally substituted with one, two, or three groups selected from R^{9b}. In some embodiments, ring B is an unsubstituted C₂₋₁₄heterocycloalkyl. In some embodiments, ring B is

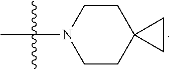 [SMILES: *N1CCC2(CC1)CC2]

In some embodiments of a compound of Formula (II), (IIa), (IIaa), (IIb), (IIbb), (IIbb′), (IIc) or (IIcc), or a pharmaceutically acceptable salt thereof, ring B is C₃₋₁₂cycloalkyl optionally substituted with one, two, or three groups selected from R^{9b}. In some embodiments, ring B is C₃₋₁₂cycloalkyl optionally substituted with one, two, or three groups selected from R^{9b}, wherein each R^{9b} is halogen. In some embodiments, ring B is an unsubstituted C₃₋₁₂cycloalkyl. In some embodiments, ring B is selected from

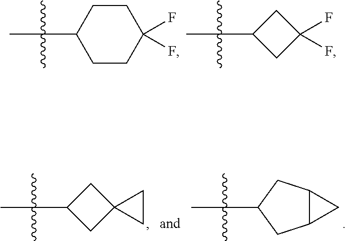 [SMILES: *C1CCC(F)(F)CC1 ; *C1CC(F)(F)C1 ; *C1CC2(CC2)C1 ; *C1CC2CC2C1]

In some embodiments, ring B is

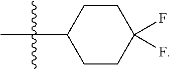 [SMILES: *C1CCC(F)(F)CC1]

In some embodiments, ring B is

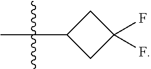 [SMILES: *C1CC(F)(F)C1]

In some embodiments, ring B is

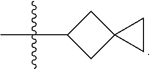 [SMILES: *C1CC2(CC2)C1]

In some embodiments, ring B is

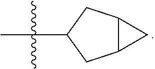 [SMILES: *C1CC2CC2C1]

In some embodiments of a compound of Formula (II), (IIa), (IIaa), (IIb), (IIbb), (IIbb′), (IIc) or (IIcc), or a pharmaceutically acceptable salt thereof, ring B is C₁₋₉heteroaryl optionally substituted with one, two, or three groups selected from R^{9b}.

In some embodiments of a compound of Formula (II), (IIa), (IIaa), (IIb), (IIbb), (IIbb′), (IIc) or (IIcc), or a pharmaceutically acceptable salt thereof, ring B is C₆₋₁₀aryl optionally substituted with one, two, or three groups selected from R^{9b}. In some embodiments, ring B is phenyl optionally substituted with one, two, or three groups selected from R^{9b}.

In some embodiments of a compound of Formula (II), (IIa), (IIaa), (IIb), (IIbb), (IIbb′), (IIc) or (IIcc), or a pharmaceutically acceptable salt thereof, R¹ is C₁₋₆alkyl optionally substituted with one, two, or three groups selected from R^{9a}. In some embodiments, R¹ is C₁₋₆alkyl optionally substituted with one, two, or three groups substituents selected from —OH and C₁₋₆alkoxy. In some embodiments, R¹ is C₁₋₆ alkylene substituted with one —OH group. In some embodiments, R¹ is —CH₂CH₂OH or —CH₂CH₂CH₂OH. In some embodiments, R¹ is —CH₂CH₂OH. In some embodiments, R¹ is —CH₂CH₂CH₂OH.

In some embodiments of a compound of Formula (II), (IIa), (IIaa), (IIb), (IIbb), (IIbb′), (IIc) or (IIcc), or a pharmaceutically acceptable salt thereof, R¹ is C₃₋₆cycloalkyl optionally substituted with one, two, or three groups selected from R^{9a}.

In some embodiments of a compound of Formula (II), (IIa), (IIaa), (IIb), (IIbb), (IIbb′), (IIc) or (IIcc), or a pharmaceutically acceptable salt thereof, R⁴ is hydrogen. In some embodiments, R⁴ is C₁₋₆alkyl.

In some embodiments, each R^{9a} is independently selected from halogen, oxo, —CN, C₁₋₆alkyl, C₃₋₆cycloalkyl, —CH₂—C₃₋₆cycloalkyl, 5-6 membered heterocycloalkyl, —CH₂-5-6 membered heterocycloalkyl, —OR¹⁰, —N(R¹⁰)(R¹¹), —C(O)OR¹⁰, —N(R¹²)S(O)₂R¹³, —C(O)R¹³, —OC(O)R¹³, —C(O)N(R¹⁰)(R¹¹), —N(R¹²)C(O)R¹³, —S(O)₂R¹³, and —S(O)₂N(R¹⁰)(R¹¹)—, wherein the alkyl, cycloalkyl, —CH₂-cycloalkyl, heterocycloalkyl, and —CH₂-heterocycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R³, —CH₂S(O)₂R¹³, —CH₂S(O)₂N(R¹⁰)(R¹¹) and —P(O)(R¹⁰)₂. In some embodiments, each R^{9a} is independently selected from halogen, oxo, amino, —CN, C₁₋₆alkyl, C₁₋₆alkoxyl, C₁₋₆haloalkyl, C₃₋₆cycloalkyl, —CH₂—C₃₋₆cycloalkyl, 5-6 membered heterocycloalkyl, and —CH₂-5-6 membered heterocycloalkyl. In some embodiments, each R^{9a} is independently selected from halogen, oxo, amino, —CN, C₁₋₆alkyl, C₁₋₆alkoxyl, and C₁₋₆haloalkyl.

In some embodiments, each R^{9b} is independently selected from halogen, oxo, —CN, C₁₋₆alkyl, C₃₋₆cycloalkyl, —CH₂—C₃₋₆cycloalkyl, 5-6 membered heterocycloalkyl, —CH₂-5-6 membered heterocycloalkyl, —OR¹⁰, —N(R¹⁰)(R¹¹), —C(O)OR¹⁰, —N(R¹²)S(O)₂R¹³, —C(O)R¹³, —OC(O)R¹³, —C(O)N(R¹⁰)(R¹¹), —N(R¹²)C(O)R¹³, —S(O)₂R¹³, and —S(O)₂N(R¹⁰)(R¹¹)—, wherein the alkyl, cycloalkyl, —CH₂-cycloalkyl, heterocycloalkyl, and —CH₂-heterocycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂. In some embodiments, each R^{9b} is independently selected from halogen, oxo, amino, —CN, C₁₋₆alkyl, C₁₋₆alkoxyl, C₁₋₆haloalkyl, C₃₋₆cycloalkyl, —CH₂—C₃₋₆cycloalkyl, 5-6 membered heterocycloalkyl, and —CH₂-5-6 membered heterocycloalkyl. In some embodiments, each R^{9b} is independently selected from halogen, oxo, amino, —CN, C₁₋₆alkyl, C₁₋₆alkoxyl, and C₁₋₆haloalkyl.

In some embodiments, each R^{9c} is independently selected from halogen, oxo, —CN, C₁₋₆alkyl, C₃₋₆cycloalkyl, —CH₂—C₃₋₆cycloalkyl, 5-6 membered heterocycloalkyl, —CH₂-5-6 membered heterocycloalkyl, —OR¹⁰, —N(R¹⁰)(R¹¹), —C(O)OR¹⁰, —N(R′)S(O)₂R¹³, —C(O)R¹³, —OC(O)R¹³, —C(O)N(R¹⁰)(R¹¹), —N(R¹²)C(O)R¹³, —S(O)₂R¹³, and —S(O)₂N(R¹⁰)(R¹¹)—, wherein the alkyl, cycloalkyl, —CH₂-cycloalkyl, heterocycloalkyl, and —CH₂-heterocycloalkyl are optionally substituted with one, two, or three groups independently selected from halogen, oxo, —CN, C₁₋₆alkyl, C₁₋₆haloalkyl, C₁₋₆alkoxy, C₁₋₆ haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R²)C(O)OR³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R′)C(O)R¹³, —CH₂S(O)₂R¹³, —CH₂S(O)₂N(R¹⁰)(R¹¹) and —P(O)(R¹⁰)₂. In some embodiments, each R^{9c} is independently selected from halogen, oxo, amino, —CN, C₁₋₆alkyl, C₁₋₆alkoxyl, C₁₋₆haloalkyl, C₃₋₆ cycloalkyl, —CH₂—C₃₋₆cycloalkyl, 5-6 membered heterocycloalkyl, and —CH₂-5-6 membered heterocycloalkyl. In some embodiments, each R^{9c} is independently selected from halogen, oxo, amino, —CN, C₁₋₆alkyl, C₁₋₆alkoxyl, and C₁₋₆haloalkyl.

In some embodiments, each R^{9d} is independently selected from halogen, oxo, —CN, C₁₋₆alkyl, C₃₋₆cycloalkyl, —CH₂—C₃₋₆cycloalkyl, 5-6 membered heterocycloalkyl, —CH₂-5-6 membered heterocycloalkyl, —OR¹⁰, —N(R¹⁰)(R¹¹), —C(O)OR¹⁰, —N(R¹²)S(O)₂R¹³, —C(O)R¹³, —OC(O)R¹¹, —C(O)N(R¹⁰)(R¹¹), —N(R¹²)C(O)R¹³, —S(O)₂R¹³, and —S(O)₂N(R¹⁰)(R¹¹)—, wherein the alkyl, cycloalkyl, —CH₂-cycloalkyl, heterocycloalkyl, and —CH₂-heterocycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂. In some embodiments, each R^{9d} is independently selected from halogen, oxo, amino, —CN, C₁₋₆alkyl, C₁₋₆alkoxyl, C₁₋₆haloalkyl, C₃₋₆cycloalkyl, —CH₂—C₃₋₆cycloalkyl, 5-6 membered heterocycloalkyl, and —CH₂-5-6 membered heterocycloalkyl. In some embodiments, each R^{9d} is independently selected from halogen, oxo, amino, —CN, C₁₋₆alkyl, C₁₋₆alkoxyl, and C₁₋₆haloalkyl.

In some embodiments, each R^{9c} is independently selected from halogen, oxo, —CN, C₁₋₆alkyl, C₃₋₆cycloalkyl, —CH₂—C₃₋₆cycloalkyl, 5-6 membered heterocycloalkyl, —CH₂-5-6 membered heterocycloalkyl, —OR¹⁰, —N(R¹⁰)(R¹¹), —C(O)OR¹⁰, —N(R¹²)S(O)₂R¹³, —C(O)R¹³, —OC(O)R¹³, —C(O)N(R¹⁰)(R¹¹), —N(R¹²)C(O)R¹³, —S(O)₂R¹³, and —S(O)₂N(R¹⁰)(R¹¹)—, wherein the alkyl, cycloalkyl, —CH₂-cycloalkyl, heterocycloalkyl, and —CH₂-heterocycloalkyl are optionally substituted with one, two, or three groups independently selected from halogen, oxo, —CN, C₁₋₆alkyl, C₁₋₆haloalkyl, C₁₋₆alkoxy, C₁₋₆ haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂. In some embodiments, each R^{9e} is independently selected from halogen, oxo, amino, —CN, C₁₋₆alkyl, C₁₋₆alkoxyl, C₁₋₆haloalkyl, C₃₋₆cycloalkyl, —CH₂—C₃₋₆cycloalkyl, 5-6 membered heterocycloalkyl, and —CH₂-5-6 membered heterocycloalkyl. In some embodiments, each R^{9e} is independently selected from halogen, oxo, amino, —CN, C₁₋₆alkyl, C₁₋₆alkoxyl, and C₁₋₆haloalkyl.

In some embodiments, each R^{9f} is independently selected from halogen, oxo, —CN, C₁₋₆alkyl, C₃₋₆cycloalkyl, —CH₂—C₃₋₆cycloalkyl, 5-6 membered heterocycloalkyl, —CH₂-5-6 membered heterocycloalkyl, —OR¹⁰, —N(R¹⁰)(R¹¹), —C(O)OR¹⁰, —N(R¹²)S(O)₂R¹³, —C(O)R¹³, —OC(O)R¹³, —C(O)N(R¹⁰)(R¹¹), —N(R¹²)C(O)R¹³, —S(O)₂R¹³, and —S(O)₂N(R¹⁰)(R¹¹)—, wherein the alkyl, cycloalkyl, —CH₂-cycloalkyl, heterocycloalkyl, and —CH₂-heterocycloalkyl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R³, —CH₂S(O)₂R¹³, —CH₂S(O)₂N(R¹⁰)(R¹¹) and —P(O)(R¹⁰)₂. In some embodiments, each R^{9f} is independently selected from halogen, oxo, amino, —CN, C₁₋₆alkyl, C₁₋₆alkoxyl, C₁₋₆haloalkyl, C₃₋₆cycloalkyl, —CH₂—C₃₋₆cycloalkyl, 5-6 membered heterocycloalkyl, and —CH₂-5-6 membered heterocycloalkyl. In some embodiments, each R^{9f} is independently selected from halogen, oxo, amino, —CN, C₁₋₆alkyl, C₁₋₆alkoxyl, and C₁₋₆haloalkyl.

In some embodiments, R^{8a} and R^{8b} are independently selected from hydrogen, halogen, C₁₋₆alkyl, and C₁₋₆haloalkyl. In some embodiments, R^{8a} is hydrogen. In some embodiments, R^{8a} is C₁₋₁₀alkyl. In some embodiments, R^{8b} is hydrogen. In some embodiments, R^{8b} is C₁₋₆alkyl.

In some embodiments, R^{8c} is selected from hydrogen, C₁₋₆alkyl, and C₁₋₆haloalkyl. In some embodiments, R^{8c} is hydrogen. In some embodiments, R^{8c} is C₁₋₆alkyl.

In some embodiments, each R^{1a} is independently selected from halogen, —CN, oxo, C₁₋₆alkyl, C₁₋₆haloalkyl, C₃₋₁₀cycloalkyl, 5-6 membered heterocycloalkyl, —OR¹⁰, —N(R¹⁰)(R¹¹), —C(O)OR¹⁰, —C(O)R¹³, —OC(O)R¹³, —C(O)N(R¹⁰)(R¹¹), and —N(R¹²)C(O)R¹³, wherein alkyl, cycloalkyl, and heterocycloalkyl are optionally substituted with one, two, or three groups selected from R^{9d}. In some embodiments, each R^{1a} is independently selected from halogen, —CN, oxo, amino, C₁₋₆alkyl, C₁₋₆haloalkyl and C₁₋₆alkoxyl.

In some embodiments, each R¹⁰ is independently selected from hydrogen, C₁₋₆alkyl, C₁₋₆haloalkyl, C₃₋₆cycloalkyl, and 5-6 membered heterocycloalkyl, wherein the alkyl, cycloalkyl, and heterocycloalkyl are optionally substituted with one, two, or three groups selected from halogen, —CN, hydroxy, C₁₋₆alkyl, C₁₋₆haloalkyl, C₁₋₆alkoxy, C₃.cycloalkyl, and C₂₋₉heterocycloalkyl. In some embodiments, each R¹⁰ is independently selected from hydrogen, C₁₋₆alkyl and C₁₋₆haloalkyl, wherein the alkyl is optionally substituted. In some embodiments, R¹⁰ is hydrogen. In some embodiments, R¹⁰ is C₁₋₃alkyl.

In some embodiments, R¹⁰ and R¹¹, together with the nitrogen to which they are attached, form a C₂₋₉heterocycloalkyl. In some embodiments, R¹⁰ and R¹¹, together with the nitrogen to which they are attached, form a 5-6 membered heterocycloalkyl.

In some embodiments, R¹ is hydrogen. In some embodiments, R¹¹ is C₁₋₆alkyl. In some embodiments, R¹¹ is C₁₋₆haloalkyl.

In some embodiments, R¹² is hydrogen. In some embodiments, R² is C₁₋₆alkyl. In some embodiments, R¹² is C₁₋₆haloalkyl.

In some embodiments, each R¹³ is independently selected C₁₋₆alkyl, C₃₋₆cycloalkyl, and 5-6 membered heterocycloalkyl, wherein the alkyl, cycloalkyl, and heterocycloalkyl are optionally substituted with one, two, or three groups selected from halogen, —CN, hydroxy, C₁₋₆alkyl, C₁₋₆haloalkyl, C₁₋₆alkoxy, C₃₋₆cycloalkyl, and C₂₋₉heterocycloalkyl. In some embodiments, each R¹³ is independently selected from C₁₋₆alkyl and C₁₋₆haloalkyl, wherein the alkyl is optionally substituted. In some embodiments, R¹³ is C₁₋₃alkyl.

Any combination of the groups described above for the various variables is contemplated herein. Throughout the specification, groups and substituents thereof are chosen by one skilled in the field to provide stable moieties and compounds.

In some embodiments, disclosed herein is a compound, or a pharmaceutically acceptable salt thereof, selected from:

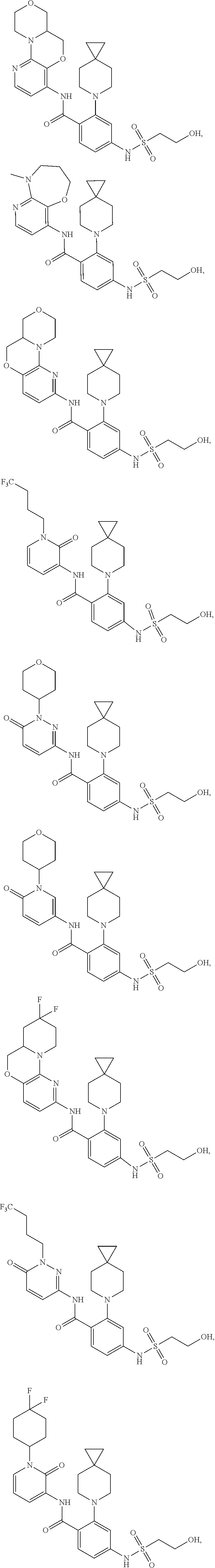

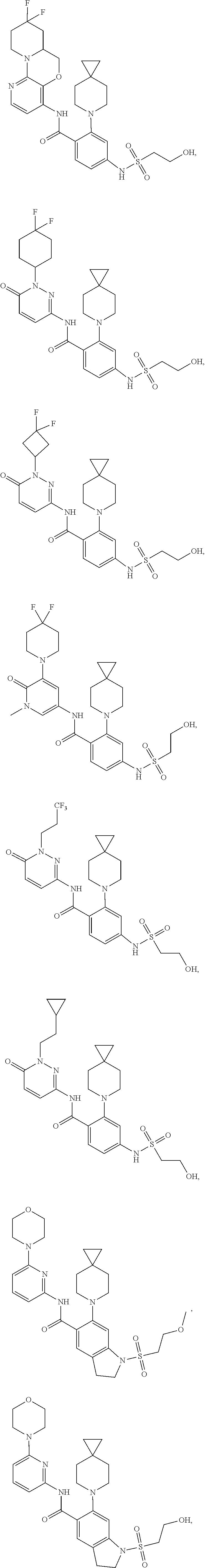

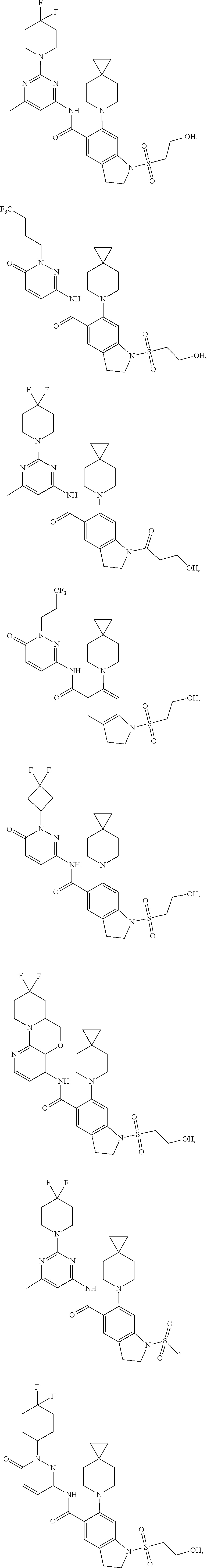

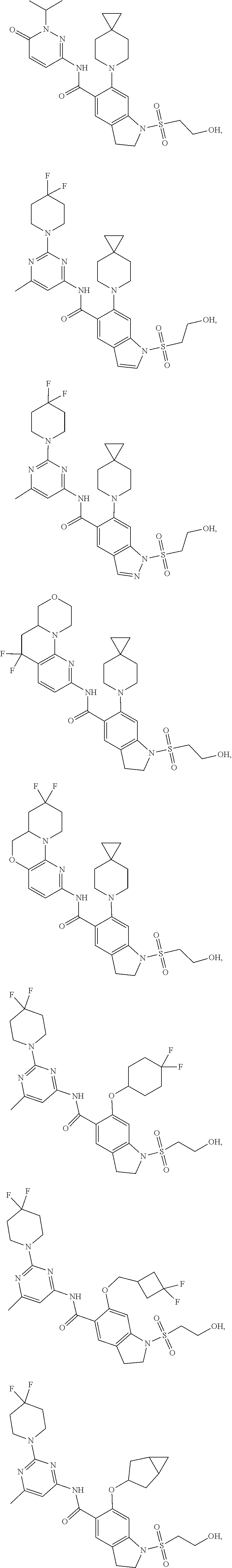

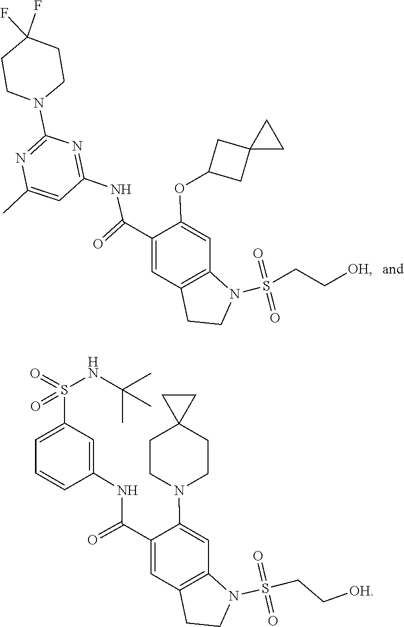 [SMILES: Cc1cc(NC(=O)c2cc3c(cc2OC2CC4(CC4)C2)N(S(=O)(=O)CCO)CC3)nc(N2CCC(F)(F)CC2)n1 ; CC(C)(C)NS(=O)(=O)c1cccc(NC(=O)c2cc3c(cc2N2CCC4(CC2)CC4)N(S(=O)(=O)CCO)CC3)c1]

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine and chloride, such as ²H (D), ³H, ³C, ⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% by molar. In some embodiments, one or more of the substituents disclosed herein comprise deuterium at a percentage higher than the natural abundance of deuterium. In some embodiments, one or more ¹H are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. In some embodiments, the disclosure provides for methods of treating diseases by administering the compounds in the form of such solvates. In some embodiments, the disclosure provides for methods of treating diseases by administering a composition comprising the compounds in the form of such solvates. Solvates contain either stoichiometric or non-stoichiometric amounts of a solvent, and, in some embodiments, are formed during the process of crystallization with pharmaceutically acceptable solvents.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein is a method of treating a disease in which inhibition of KIF18A is beneficial, the method comprising administering a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc) disclosed herein, or a pharmaceutically acceptable salt thereof.

Disclosed herein is a method of treating a disease or disorder associated with KIF18A, the method comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc) disclosed herein, or a pharmaceutically acceptable salt thereof.

Disclosed herein is a method of treating cancer in a subject, the method comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc) disclosed herein, or a pharmaceutically acceptable salt thereof. In some embodiments of a method of treating cancer in a subject, the method comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc) disclosed herein, or a pharmaceutically acceptable salt thereof, wherein the cancer is selected from the group consisting of (a) a solid or hematologically derived tumor selected from cancer of the bladder, endometrial, lung squamous cell, breast, colon, kidney, liver, lung, small cell lung cancer, esophagus, gallbladder, brain, head and neck, ovary, pancreas, stomach, cervix, thyroid, prostate, and skin; (b) a hematopoietic tumor of lymphoid lineage selected from leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, and Burkett's lymphoma; (c) a hematopoietic tumor of myeloid lineage selected from acute and chronic myelogenous leukemias, myelodysplastic syndrome and promyelocytic leukemia; (d) a tumor of mesenchymal origin selected from fibrosarcoma and rhabdomyosarcoma; (e) a tumor of the central and peripheral nervous system selected from astrocytoma, neuroblastoma, glioma, and schwannoma; and (f) a melanoma, seminoma, teratocarcinoma, osteosarcoma, xenoderoma pigmentosum, keratoctanthoma, thyroid follicular cancer or Karposi's sarcoma. In some embodiments of a method of treating a solid or hematologically derived tumor selected from cancer of the bladder, endometrial, lung squamous cell, breast, colon, kidney, liver, lung, small cell lung cancer, esophagus, gallbladder, brain, head and neck, ovary, pancreas, stomach, cervix, thyroid, prostate, and skin in a subject, the method comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc) disclosed herein, or a pharmaceutically acceptable salt thereof. In some embodiments of a method of treating a hematopoietic tumor of lymphoid lineage selected from leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, and Burkett's lymphoma in a subject, the method comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc) disclosed herein, or a pharmaceutically acceptable salt thereof. In some embodiments of a method of treating a hematopoietic tumor of myeloid lineage selected from acute and chronic myelogenous leukemias, myelodysplastic syndrome and promyelocytic leukemia in a subject, the method comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc) disclosed herein, or a pharmaceutically acceptable salt thereof. In some embodiments of a method of treating a tumor of mesenchymal origin selected from fibrosarcoma and rhabdomyosarcoma in a subject, the method comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc) disclosed herein, or a pharmaceutically acceptable salt thereof. In some embodiments of a method of treating a tumor of the central and peripheral nervous system selected from astrocytoma, neuroblastoma, glioma, and schwannoma in a subject, the method comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc) disclosed herein, or a pharmaceutically acceptable salt thereof. In some embodiments of a method of treating a melanoma, seminoma, teratocarcinoma, osteosarcoma, xenoderoma pigmentosum, keratoctanthoma, thyroid follicular cancer or Karposi's sarcoma in a subject, the method comprising administering to the subject a compound of Formula (I), (Ia), (Ib), (Ic), (Id), (Ie), (If), (Ig), (Iaa), (Ibb), (Icc), (Idd), (Iee), (Iff), (Igg), (II), (IIa), (IIb), (IIc), (IIaa), (IIbb), (IIbb′), or (IIcc) disclosed herein, or a pharmaceutically acceptable salt thereof.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In various embodiments, the dosages are altered depending on a number of variables including, but not limited to, the activity of the compound used, the disease or condition to be treated, the mode of administration, the requirements of the individual subject, the severity of the disease or condition being treated, and the judgment of the practitioner.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

### EXAMPLES

The following examples are provided for illustrative purposes only and not to limit the scope of the claims provided herein.

As used above, and throughout the description of the invention, the following abbreviations, unless otherwise indicated, shall be understood to have the following meanings:
ACN or MeCN acetonitrile
AcOH acetic acid
Ac acetyl
Bn benzyl
BOC or Boc tert-butyl carbamate
i-Bu iso-butyl
t-Bu tert-butyl
CDI 1,1-carbonyldiimidazole
DBU 1,8-diazabicyclo[5.4.0]undec-7-ene
DCE dichloroethane (ClCH₂CH₂Cl)
DCM dichloromethane (CH₂Cl₂)
DIBAL-H diisobutylaluminum hydride
DIPEA or DIEA diisopropylethylamine
DMAP 4-(N,N-dimethylamino)pyridine
DME 1,2-dimethoxyethane
DMF N,N-dimethylformamide
DMA N,N-dimethylacetamide
DMPU N,N′-dimethylpropyleneurea
DMSO dimethylsulfoxide
DPPA diphenyl phosphoryl azide
Dppf or dppf 1,1′-bis(diphenylphosphino)ferrocene
EDC or EDCI N-(3-dimethylaminopropyl)-N′-ethylcarbodiimide hydrochloride
eq equivalent(s)
Et ethyl
Et₂O diethyl ether
EtOH ethanol
EA or EtOAc ethyl acetate
HATU 1-[bis(dimethylamino)methylene]-1H-1,2,3-triazolo[4,5-b]pyridinium 3-oxid hexafluorophosphate
HOBt 1-hydroxybenzotriazole
HPLC high performance liquid chromatography
KOAc potassium acetate
KOtBu potassium tert-butoxide
KHMDS potassium bis(trimethylsilyl)amide
NaHMDS sodium bis(trimethylsilyl)amide
LiHMDS lithium bis(trimethylsilyl)amide
LAH/LiAlH₄ lithium aluminum anhydride
LCMS liquid chromatography mass spectrometry
Me methyl
MeOH methanol
MS mass spectroscopy
MTBE methyl tert-butyl ether
NBS N-bromosuccinimide
NMP N-methyl-pyrrolidin-2-one
NMR nuclear magnetic resonance
PE petroleum ether
Ph phenyl
iPr/i-Pr iso-propyl
PyAOP 7-azabenzotriazol-1-yloxy)tripyrrolidinophosphonium hexafluorophosphate
RP-HPLC reverse-phase high-pressure liquid chromatography
rt room temperature
SEM 2-(trimethylsilyl)ethoxymethyl
TBS tert-butyldimethylsilyl
TEA triethylamine
TFA trifluoroacetic acid
THF tetrahydrofuran
TLC thin layer chromatography
TMS trimethylsilyl

### Intermediate 01: Synthesis of 4-iodo-2-(6-azaspiro[2.5]octan-6-yl)benzoic acid (INT01)

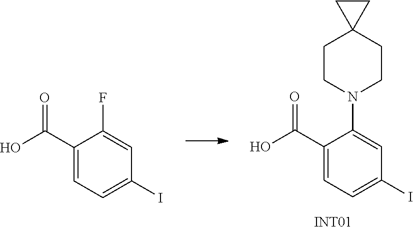 [SMILES: O=C(O)c1ccc(I)cc1F ; O=C(O)c1ccc(I)cc1N1CCC2(CC1)CC2 ; "INT01"]

### Step 1

To a solution of 2-fluoro-4-iodobenzoic acid (1 g, 3.76 mmol), 6-azaspiro[2.5]octane (0.71 g, 4.89 mmol) in DMSO (5 mL) was added K₂CO₃ (1.56 g, 11.28 mmol). The mixture was stirred at 140° C. for 18 hrs under N₂ atmosphere. The reaction was cooled to rt, diluted with water (100 mL), and washed with PE (100 mL×3). The aqueous phase was separated and acidified with HCl (1 M) to pH=6. The aqueous solution was concentrated under reduced pressure to afford INT01 (700 mg). LCMS [M+H]⁺: 358.0.

### Intermediate 02: Synthesis of 1-((2-methoxyethyl)sulfonyl)-6-(6-azaspiro[2.5]octan-6-yl)indoline-5-carboxylic acid (INT 02)

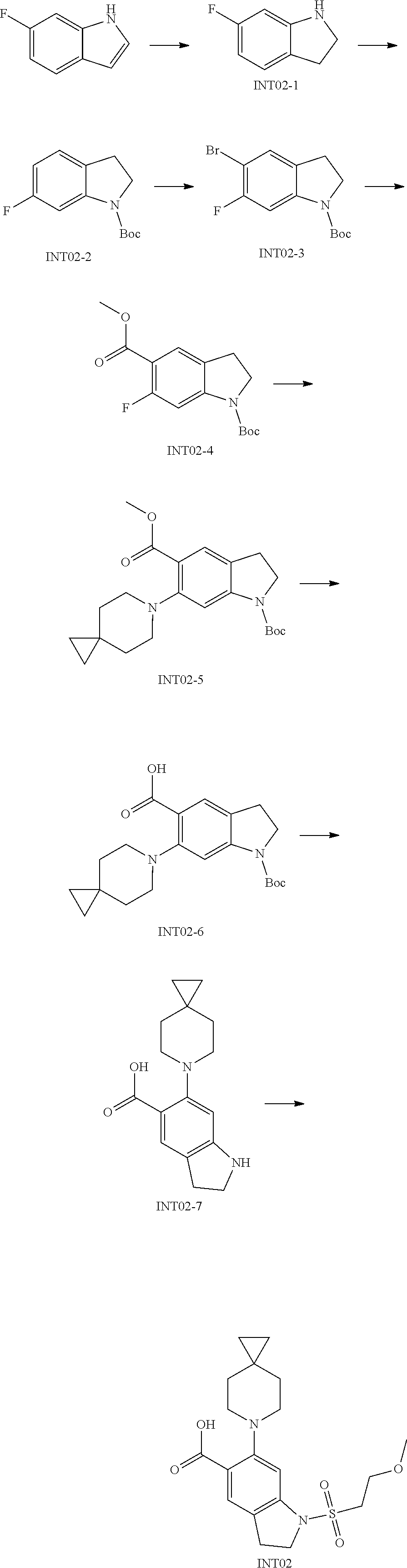

### Step 1

To a solution of 6-fluoro-1H-indole (20.0 g, 148 mmol) in AcOH (60 mL) was added NaBH₃CN (18.6 g, 296 mmol). The mixture was stirred at 25° C. for 2 hrs. The reaction mixture was washed with aqueous NaOH (2 M, 1.5 L) and extracted with DCM (400 mL×3). The combined organic layer was washed with brine (500 mL×2), dried over anhydrous sodium sulfate, and concentrated under reduced pressure. The obtained residue was purified by silica gel chromatography to afford INT02-1 (14.3 g). LCMS [M+H]⁺: 138.3.

### Step 2

To a solution of INT02-1 (14.3 g, 104 mmol) in DCM (100 mL) were added TEA (29 mL, 209 mmol), DMAP (2.55 g, 20.9 mmol) and (Boc)₂O (35.9 mL, 156.4 mmol). The mixture was stirred at 25° C. for 24 hrs. The reaction mixture was washed with water (60 mL) and extracted with DCM (5 mL×3). The combined organic layer was washed with brine (8 mL×2), dried over anhydrous sodium sulfate, and concentrated under reduced pressure. The obtained residue was purified by silica gel chromatography to afford INT02-2 (22.0 g).

### Step 3

To a solution of INT02-2 (14.3 g, 60.3 mmol) in DMF (150 mL) was added NBS (12.9 g, 72.3 mmol). The mixture was stirred at 25° C. for 12 hrs. The reaction mixture was diluted with water (300 mL), filtered, and concentrated under reduced pressure. The obtained residue was purified by silica gel chromatography to afford INT02-3 (16.5 g).

### Step 4

To a solution of INT02-3 (7.00 g, 22.1 mmol), NaOAc (5.45 g, 66.4 mmol) in DMF (70 mL) and MeOH (70 mL) was added Pd(dppf)Cl₂ (3.24 g, 4.43 mmol) under N₂ atmosphere. The suspension was degassed and purged with CO for 3 times. The mixture was stirred under CO atmosphere (50 Psi) at 80° C. for 24 hrs. The reaction mixture was filtered and concentrated under reduced pressure. The obtained residue was purified by silica gel chromatography to afford INT02-4 (5.5 g).

### Step 5

To a solution of INT02-4 (2.00 g, 6.77 mmol), K₂CO₃ (4.68 g, 33.9 mmol) in DMSO (40 mL) was added 6-azaspiro[2.5]octane (3.77 g, 33.9 mmol). The mixture was stirred at 130° C. for 12 hrs under N₂ atmosphere. The reaction mixture was diluted with water (40 mL), filtered, and concentrated under reduced pressure to afford INT02-5 (725 mg). LCMS [M+H]⁺: 387.3.

### Step 6

To a solution of INT02-5 (700 mg, 1.81 mmol) in THF (15 mL) was added LiOH (4.53 mL, 9.06 mmol) in water (2 mL). The mixture was stirred at 85° C. for 12 hrs. The mixture was cooled to 25° C. and washed with water (20 mL). The aqueous phase was acidified to pH=1 with 1 M HCl and extracted with EtOAc (35 mL×3). The combined organic phase was dried over anhydrous sodium sulfate and concentrated under reduced pressure to afford INT02-6 (800 mg). LCMS [M+H]⁺: 371.2.

### Step 7

A solution of INT02-6 (1.00 g, 2.68 mmol) in HCl/dioxane (10 mL) was stirred at 25° C. for 3 hrs. The reaction mixture was concentrated under reduced pressure to afford INT02-7 (800 mg). LCMS [M+H]⁺: 273.2.

### Step 8

To a solution of INT02-7 (800 mg, 2.93 mmol) in DCM (2 mL) were added DIEA (2.91 mL, 17.6 mmol) and 2-methoxyethane-1-sulfonyl chloride (931 mg, 5.87 mmol). The mixture was stirred at 25° C. for 3 hrs. The reaction mixture was concentrated under reduced pressure and purified by prep-HPLC to afford INT02 (130 mg). LCMS [M+H]⁺: 395.1.

### Example 1: 4-((2-hydroxyethyl)sulfonamido)-2-(6-azaspiro[2.5]octan-6-yl)-N-(6a,7,9,10-tetrahydro-6H-[1,4]oxazino[4,3-d]pyrido[3,2-b][1,4]oxazin-4-yl)benzamide (EX01)

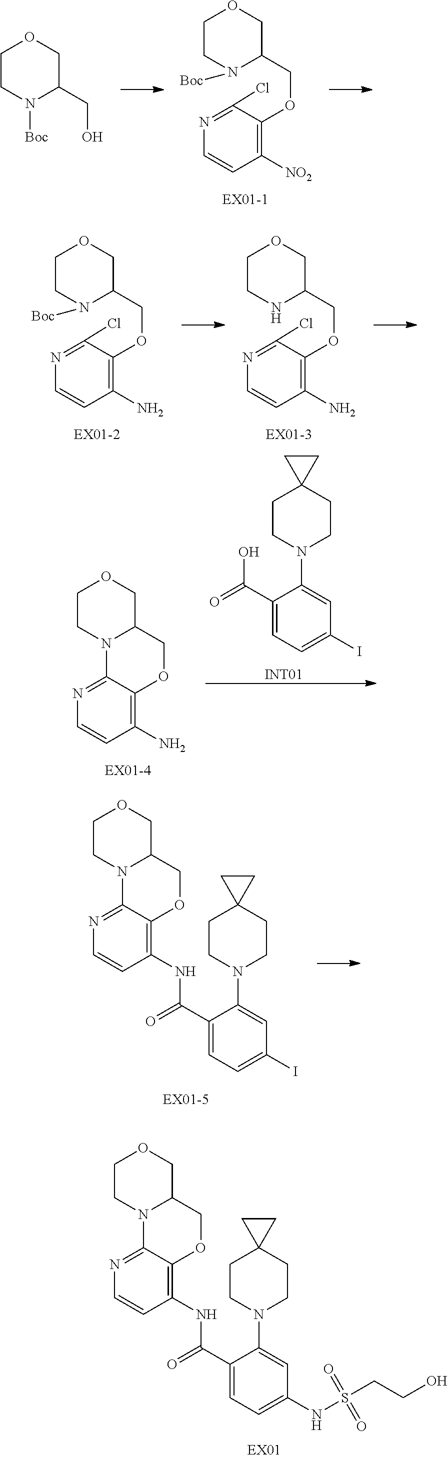

### Step 1

To a solution of tert-butyl 3-(hydroxymethyl)morpholine-4-carboxylate (123 mg, 0.566 mmol) in THF (5 mL) was added NaH (45 mg, 1.13 mmol). The reaction was stirred at 0° C. for 1 hr, followed by the addition of 2-chloro-3-fluoro-4-nitropyridine (100 mg, 0.566 mmol) in THF (5 mL). The resulting mixture was stirred at 0° C. for 12 hrs. The reaction was quenched by aqueous NH₄Cl (10 mL) and extracted with EtOAc (20 mL×2). The organic layer was dried over anhydrous sodium sulfate and concentrated under reduced pressure. The residue was purified by silica gel chromatography to afford EX01-1 (580 mg). LCMS [M+Na]⁺: 396.0.

### Step 2

To a mixture of EX01-1 (420 mg, 1.12 mmol) and NH₄Cl (420 mg, 7.86 mmol) in EtOH (4 mL) and H₂O (4 mL) was added iron powder (439 mg, 7.86 mmol) in three portions. The resulting mixture was stirred at 80° C. for 3 hrs. The reaction was cooled to rt, and filter through Celite. The filtrate was concentrated under reduced pressure, and then diluted with EtOAc (50 mL) and H₂O (100 mL). The organic layer was separated and dried over anhydrous sodium sulfate. The organic phase was concentrated under reduced pressure and purified by silica gel chromatography to afford EX01-2 (360 mg). LCMS [M+H]⁺: 344.2.

### Step 3

To a solution of EX01-2 (360 mg, 1.04 mmol) in DCM (10 mL) was added HCl/MeOH (10 mL). The reaction mixture was stirred at 25° C. for 2 hrs. The mixture was concentrated under reduced pressure to afford EX01-3 (351 mg). LCMS [M+H]⁺: 244.1.

### Step 4

To a solution of EX01-3 (50.0 mg, 0.183 mmol) in dioxane (20 mL) were added Cs₂CO₃ (1.20 mg, 3.69 mmol), Xantphos (142 mg, 0.246 mmol) and Pd₂(dba)₃ (112 mg, 0.123 mmol) under N₂ atmosphere. The reaction was stirred at 80° C. for 2 hrs under N₂ atmosphere. The reaction was cooled to rt, diluted with water (100 mL) and extracted with EtOAc (50 mL×2). The organic layer was dried over anhydrous sodium sulfate, concentrated under reduced pressure, and purified by silica gel chromatography to afford EX01-4 (140 mg). LCMS [M+H]⁺: 208.2.

### Step 5

To a solution of EX01-4 (189 mg, 0.531 mmol) and INT01 (110 mg, 0.531 mmol) in DCM (5 mL) were added pyridine (0.43 mL, 5.30 mmol) and POCl₃ (0.15 mL, 1.59 mmol) at 0° C. The reaction was stirred at 25° C. for 2 hr. The reaction mixture was quenched with water (20 mL) at 25° C. and extracted with EtOAc (30 mL×3). The combined organic layer was concentrated under reduced pressure and purified by prep-TLC to afford EX01-5 (110 mg). LCMS[M+H]⁺: 547.3.

### Step 6

A mixture of EX01-5 (100 mg, 0.183 mmol), 2-hydroxyethane-1-sulfonamide (23 mg, 0.183 mmol), K₃PO₄ (116 mg, 0.549 mmol), CuI (35 mg, 0.183 mmol) and sarcosine (26 mg, 0.183 mmol) in DMF (2 mL) was stirred at 110° C. for 1 hr under N₂ atmosphere. The mixture was filtered, concentrated under reduced pressure, and purified by prep-HPLC to afford EX01 (44.4 mg). ¹H NMR (400 MHz, CD₃OD) δ 8.03 (d, J=8.6 Hz, 1H), 7.98 (d, J=5.8 Hz, 1H), 7.69 (d, J=5.7 Hz, 1H), 7.27 (d, J=2.2 Hz, 1H), 7.09 (dd, J=8.6, 2.1 Hz, 1H), 4.43 (dd, J=10.8, 2.9 Hz, 1H), 4.23 (dd, J=12.8, 2.3 Hz, 1H), 4.07-3.99 (m, 2H), 3.98-3.91 (m, 3H), 3.65 (td, J=11.8, 2.8 Hz, 1H), 3.57-3.46 (m, 1H), 3.38-3.32 (m, 3H), 3.03 (q, J=5.0 Hz, 4H), 2.93 (td, J=12.5, 3.6 Hz, 1H), 1.65 (s, 4H), 0.41 (s, 4H). LCMS [M+H]⁺: 544.2.

### Example 2: 4-((2-hydroxyethyl)sulfonamido)-N-(5-methyl-2,3,4,5-tetrahydropyrido[3,2-b][1,4]oxazepin-9-yl)-2-(6-azaspiro[2.5]octan-6-yl)benzamide (EX02)

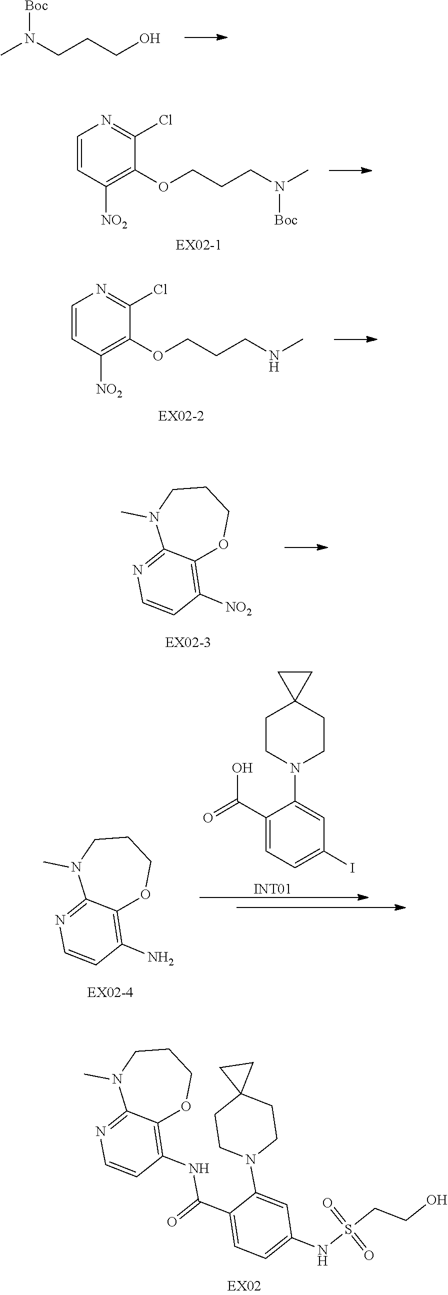

### Step 1

To a solution of tert-butyl (3-hydroxypropyl)(methyl)carbamate (3.00 g, 15.9 mmol) and 2-chloro-4-nitropyridin-3-ol (3.04 g, 17.4 mmol) and PPh₃ (6.24 g, 23.8 mmol) in THF (30 mL) was slowly added DIAD (4.68 mL, 23.8 mmol) at 0° C. The reaction was stirred at 25° C. for 3 hrs. The reaction mixture was poured into water (80 mL) and extracted with EtOAc (30 mL×3). The organic layer was washed with brine (20 mL×2), dried over anhydrous sodium sulfate, concentrated under reduced pressure, and purified by silica gel chromatography to afford EX02-1 (5.1 g).

To a solution of EX02-1 (5.10 g, 14.7 mmol) in DCM (50 mL) was added TFA (10 mL, 14.7 mmol). The reaction was stirred at 25° C. for 2 hrs. The reaction mixture was concentrated under reduced pressure to afford EX02-2 (6.0 g). LCMS[M+H]⁺: 246.3.

### Step 2

To a solution of EX02-2 (1.50 g, 6.11 mmol), XPhosPd G3 (0.78 g, 0.92 mmol) in dioxane (20 mL) was added Cs₂CO₃ (15.9 g, 48.8 mmol) under N₂ atmosphere. The reaction was stirred at 100 TC for 6 hrs under N₂ atmosphere. The reaction mixture was poured into the water (200 mL) and extracted with EtOAc (40 mL×4). The combined organic layer was washed with brine (100 mL×2), dried over anhydrous sodium sulfate, concentrated under reduced pressure, and purified by silica gel chromatography to afford EX02-3 (550 mg). LCMS[M+H]⁺: 210.2.

### Step 3

To a suspension of Pd/C (10 wt %, 0.61 g, 5.74 mmol) in MeOH (10 mL) was added EX02-3 (0.60 g, 2.87 mmol). The mixture was stirred at 25° C. for 4 hrs under H₂ atmosphere. The reaction mixture was filtered, and concentrated under reduced pressure to give EX02-4 (400 mg). LCMS[M+H]⁺: 180.2.

### Step 4

EX02 (15 mg) was prepared as described in Example 1 (EX01) Steps 5-6, except EX02-4 was used instead of EX01-4 in Step 5. H NMR (400 MHz, CD₃OD) δ 7.96 (d, J=5.7 Hz, 1H), 7.91 (d, J=8.6 Hz, 1H), 7.73 (d, J=5.7 Hz, 1H), 7.15 (d, J=2.1 Hz, 1H), 6.98 (dd, J=8.6, 2.1 Hz, 1H), 4.35 (t, J=6.1 Hz, 2H), 3.96 (t, J=6.6 Hz, 2H), 3.55-3.48 (m, 3H), 3.31-3.28 (m, 2H), 3.07 (d, J=6.1 Hz, 7H), 2.15 (p, J=6.0 Hz, 2H), 1.67 (brs, 4H), 0.40 (s, 4H). LCMS [M+H]⁺: 516.2.

### Example 3: 4-((2-hydroxyethyl)sulfonamido)-2-(6-azaspiro[2.5]octan-6-yl)-N-(6a,7,9,10-tetrahydro-6H-[1,4]oxazino[4,3-d]pyrido[3,2-b][1,4]oxazin-2-yl)benzamide (EX03)

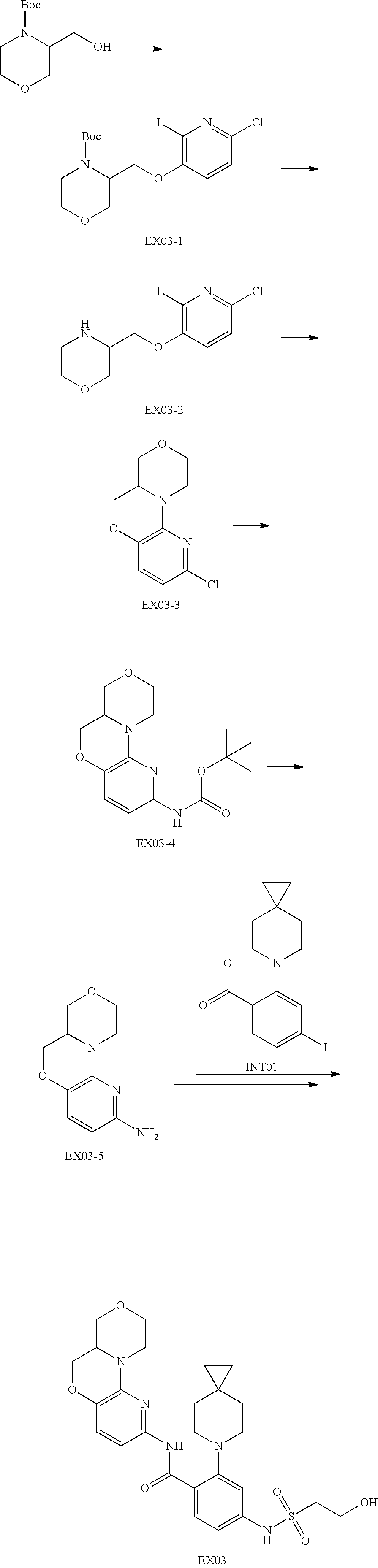

### Step 1

To a solution of 6-chloro-2-iodopyridin-3-ol (2.35 g, 9.21 mmol), PPh₃ (3.62 g, 13.81 mmol) and tert-butyl 3-(hydroxymethyl)morpholine-4-carboxylate (2.00 g, 9.21 mmol) in THF (40 mL) was slowly added a solution of DIAD (2.79 g, 13.8 mmol, 2.68 mL) in THF (40 mL) at 0° C. under N₂ atmosphere. The mixture was stirred at 20° C. for 16 hrs under N₂ atmosphere. The reaction mixture was filtered, and the filtrate was concentrated under reduced pressure. The obtained residue was purified by prep-HPLC to afford EX03-1 (600 mg). LCMS [M+H]⁺: 455.7.

### Step 2

A mixture of EX03-1 (600 mg, 1.32 mmol) and HCl/MeOH (4 M, 1.65 mL) in MeOH (6 mL) was stirred at 25° C. for 2 hrs under N₂ atmosphere. The mixture was filtered, and the filtrate was concentrated under reduced pressure to afford EX03-2 (450 mg). LCMS[M+H]⁺: 355.0.

### Step 3

A mixture of EX03-2 (450 mg, 1.83 mmol), CuI (69.8 mg, 367 umol) and K₃PO₄ (2.18 g, 10.27 mmol) in DMSO (10 mL) was degassed and purged with N₂ for 3 times. The mixture was stirred at 100° C. for 3 hrs under N₂ atmosphere. The mixture was filtered, and the filtrate was diluted with water (10 mL) and extracted with EtOAc (30 mL×3). The combined organic layer was washed with brine (5 mL×2), dried over anhydrous sodium sulfate, and concentrated under reduced pressure to give EX03-3 (350 mg). LCMS[M+H]⁺: 227.1.

### Step 4

A mixture of EX03-3 (400 mg, 1.76 mmol), tert-butyl carbamate (289 mg, 2.47 mmol), Cs₂CO₃ (1.72 g, 5.29 mmol), Pd₂(dba)₃ (323 mg, 353 umol) and dicyclohexyl-[2-(2,4,6-triisopropylphenyl)phenyl]phosphane (336 mg, 706 umol) in toluene (5 mL) was degassed and purged with N₂ for 3 times. The mixture was stirred at 85° C. for 12 hrs under N₂ atmosphere. The mixture was filtered, and the filtrate was diluted with water (15 mL) and extracted with EtOAc (30 mL×3). The combined organic layer was washed with brine (5 mL×2), dried over anhydrous sodium sulfate, and concentrated under reduced pressure. The obtained residue was purified by prep-HPLC to afford EX03-4 (180 mg). LCMS[M+H]⁺: 308.2.

### Step 5

A mixture of EX03-4 (180 mg, 0.781 mmol) in DCM (2 mL) and TFA (89 mg, 0.78 mmol) was stirred at 25° C. for 3 hrs under N₂ atmosphere. The mixture was filtered, and the filtrate was concentrated under reduced pressure and purified by prep-HPLC to afford EX03-5 (160 mg). LCMS[M+Na]⁺: 230.3.

### Step 6

EX03 (77 mg) was prepared as described in Example 1 (EX01) Steps 5-6, except EX03-5 was used instead of EX01-4 in Step 5. ¹H NMR (400 MHz, DMSO-d₆) δ 13.09 (s, 1H), 10.16 (s, 1H), 8.06 (d, J=8.6 Hz, 1H), 7.53 (d, J=8.4 Hz, 1H), 7.26 (d, J=2.2 Hz, 1H), 7.11 (dd, J=8.6, 2.2 Hz, 1H), 7.06 (d, J=8.3 Hz, 1H), 4.93 (s, 1H), 4.28-4.18 (m, 2H), 3.98 (dd, J=11.4, 3.4 Hz, 1H), 3.93-3.83 (m, 2H), 3.75 (t, J=6.6 Hz, 2H), 3.57 (td, J=11.8, 2.7 Hz, 1H), 3.49-3.41 (m, 1H), 3.35 (t, J=6.5 Hz, 2H), 3.23-3.13 (m, 2H), 2.96 (t, J=5.2 Hz, 4H), 1.75 (s, 4H), 0.37 (s, 4H). LCMS [M+H]⁺: 544.4.

### Example 4: 4-((2-hydroxyethyl)sulfonamido)-N-(2-oxo-1-(4,4,4-trifluorobutyl)-1,2-dihydropyridin-3-yl)-2-(6-azaspiro[2.5]octan-6-yl)benzamide (EX04)

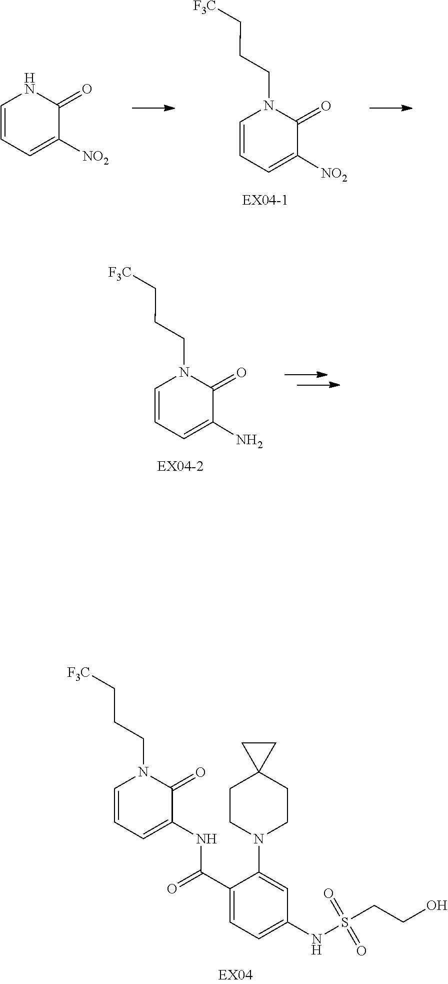 [SMILES: O=c1[nH]cccc1[N+](=O)[O-] ; O=c1c([N+](=O)[O-])cccn1CCCC(F)(F)F ; Nc1cccn(CCCC(F)(F)F)c1=O ; O=C(Nc1cccn(CCCC(F)(F)F)c1=O)c1ccc(NS(=O)(=O)CCO)cc1N1CCC2(CC1)CC2]

### Step 1

To a solution of 3-nitropyridin-2(1H)-one (1.40 g, 9.99 mmol) in DMF (50 mL) were added Cs₂CO₃ (6.51 g, 19.98 mmol) and 4-bromo-1,1,1-trifluorobutane (2.00 g, 10.47 mmol). The reaction was stirred at 70° C. for 5 hrs. The reaction mixture was cooled to rt, quenched with aqueous NH₄Cl (10 mL), and extracted with EtOAc (10 mL×3). The combined organic layer was washed with brine (20 mL), dried over anhydrous sodium sulfate, and concentrated under reduced pressure. The residue was purified by silica gel chromatography to afford EX04-1 (500 mg). ¹H NMR (400 MHz, DMSO-d₆) δ 8.40 (dd, J=7.6, 2.0 Hz, 1H), 8.21 (dd, J=6.8, 2.0 Hz, 1H), 6.45 (dd, J=7.6, 6.63 Hz, 1H), 4.09 (t, J=7.2 Hz, 2H), 2.26-2.38 (m, 2H), 1.87-1.95 (m, 2H). LCMS[M+H]⁺: 251.1.

### Step 2

To a stirred solution of EX04-1 (500 mg, 2.00 mmol) in EtOH (20 mL) was added a solution NH₄Cl (321 mg, 6.00 mmol) in water (4 mL), followed by the addition of iron powder (558 mg, 10.0 mmol) in portions. The mixture was stirred at 50° C. for 5 hrs. The mixture was cooled to rt, filtered through Celite and washed with EtOAc (20 mL). The filtrate was concentrated under reduced pressure to afford EX04-2 (440 mg). LCMS[M+H]⁺: 221.2.

### Step 3

EX04 (62 mg) was prepared as described in Example 1 (EX01) Steps 5-6, except EX04-2 was used instead of EX01-4 in Step 5. ¹H NMR (400 MHz, CD₃OD) δ 8.60 (dd, J=7.6, 1.8 Hz, 1H), 7.96 (d, J=8.6 Hz, 1H), 7.33 (dd, J=6.9, 1.8 Hz, 1H), 7.23 (d, J=2.2 Hz, 1H), 7.07 (dd, J=8.6, 2.1 Hz, 1H), 6.40 (t, J=7.2 Hz, 1H), 4.16 (t, J=7.1 Hz, 2H), 3.94 (t, J=6.3 Hz, 2H), 3.36 (t, J=6.3 Hz, 2H), 3.03 (t, J=5.4 Hz, 4H), 2.36-2.17 (m, 2H), 2.13-1.99 (m, 2H), 1.69 (s, 4H), 0.34 (s, 4H). LCMS [M+H]⁺: 557.3.

### Example 5: 4-((2-hydroxyethyl)sulfonamido)-N-(6-oxo-1-(tetrahydro-2H-pyran-4-yl)-1,6-dihydropyridazin-3-yl)-2-(6-azaspiro[2.5]octan-6-yl)benzamide (EX05)

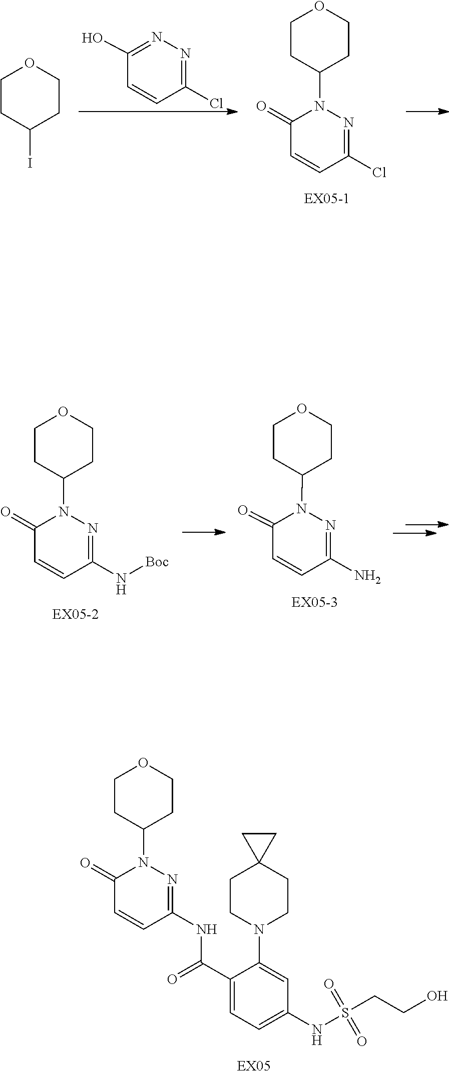 [SMILES: IC1CCOCC1 ; Oc1ccc(Cl)nn1 ; O=c1ccc(Cl)nn1C1CCOCC1 ; CC(C)(C)OC(=O)Nc1ccc(=O)n(C2CCOCC2)n1 ; Nc1ccc(=O)n(C2CCOCC2)n1 ; O=C(Nc1ccc(=O)n(C2CCOCC2)n1)c1ccc(NS(=O)(=O)CCO)cc1N1CCC2(CC1)CC2]

### Step 1

To a solution of 6-chloropyridazin-3-ol (1.00 g, 7.66 mmol) and 4-iodotetrahydro-2H-pyran (2.27 g, 10.7 mmol) in DMF (10 mL) was added K₂CO₃ (2.12 g, 15.3 mmol). The reaction was stirred at 80° C. for 2 hrs. The reaction mixture was cooled to rt, poured into the water (100 mL), and extracted with EtOAc (30 mL×4). The combined organic layer was washed with brine (50 mL×2), dried over anhydrous sodium sulfate, and filtered. The filtrate was concentrated under reduced pressure and purified by prep-TLC to afford EX05-1 (520 mg). LCMS[M+H]⁺: 215.2.

### Step 2

To a suspension of EX05-1 (500 mg, 2.33 mmol), tert-butyl carbamate (546 mg, 4.66 mmol), Pd₂(dba)₃ (427 mg, 0.47 mmol) and XPhos (444 mg, 0.93 mmol) in toluene (15 mL) was added Cs₂CO₃ (2276 mg, 6.99 mmol). The reaction was stirred at 90° C. for 12 hrs under N₂. The reaction mixture was poured into the water (60 mL) and extracted with EtOAc (30 mL×2). The combined organic layer was washed with brine (40 mL×2), dried over anhydrous sodium sulfate, and filtered. The filtrate was concentrated under reduced pressure and purified by silica gel chromatography to afford EX05-2 (600 mg). LCMS[M+H]⁺: 296.2.

### Step 3

To a solution of EX05-2 (500 mg, 1.69 mmol) in dioxane (10 mL) was added HCl (2 mL, 1.69 mmol). The reaction was stirred at 25° C. for 2 hrs. The reaction mixture was poured into the water (50 mL), extracted with DCM/MeOH (20:1, 30 mL×4). The combined organic layer was dried over anhydrous sodium sulfate, concentrated under reduced pressure, and purified by silica gel chromatography to afford EX05-3 (320 mg). LCMS[M+H]⁺: 196.3.

### Step 4

EX05 (40 mg) was prepared as described in Example 1 (EX01) Steps 5-6, except EX05-3 was used instead of EX01-4 in Step 5. ¹H NMR (400 MHz, CD₃OD) δ 8.55 (d, J=9.8 Hz, 1H), 8.15 (d, J=8.6 Hz, 1H), 7.35 (d, J=2.1 Hz, 1H), 7.19 (dd, J=8.7, 2.2 Hz, 1H), 7.09 (d, J=9.9 Hz, 1H), 5.16 (tt, J=11.6, 3.8 Hz, 1H), 4.11 (dd, J=11.8, 4.5 Hz, 2H), 3.97 (t, J=6.2 Hz, 2H), 3.63 (t, J=11.9 Hz, 2H), 3.39 (t, J=6.3 Hz, 2H), 3.19-3.04 (m, 4H), 2.19 (qd, J=12.2, 4.3 Hz, 2H), 2.01-1.60 (m, 6H), 0.49 (s, 4H). LCMS [M+H]⁺: 532.4.

### Example 6: 4-((2-hydroxyethyl)sulfonamido)-N-(6-oxo-1-(tetrahydro-2H-pyran-4-yl)-1,6-dihydropyridin-3-yl)-2-(6-azaspiro[2.5]octan-6-yl)benzamide (EX06)

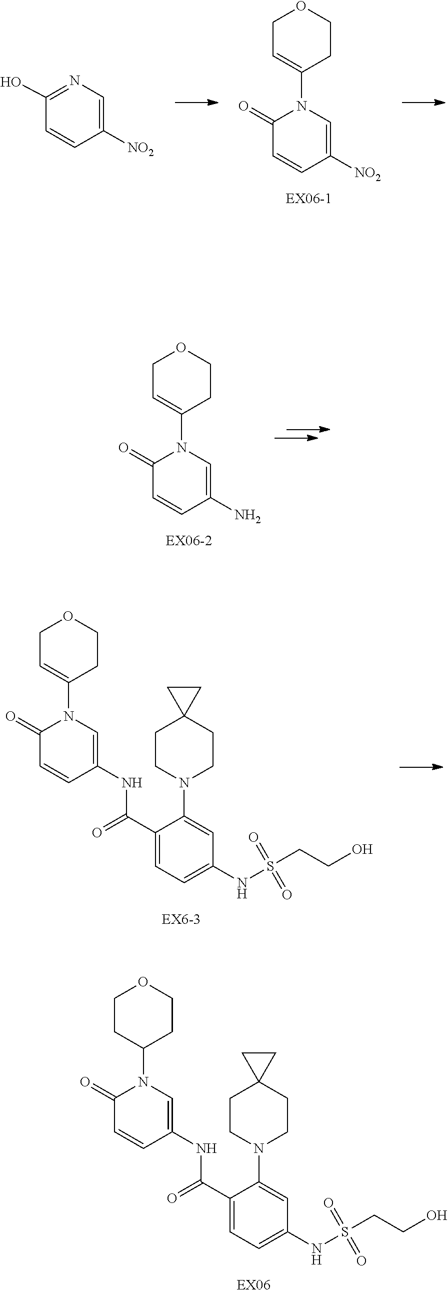 [SMILES: O=[N+]([O-])c1ccc(O)nc1 ; O=c1ccc([N+](=O)[O-])cn1C1=CCOCC1 ; Nc1ccc(=O)n(C2=CCOCC2)c1 ; O=C(Nc1ccc(=O)n(C2=CCOCC2)c1)c1ccc(NS(=O)(=O)CCO)cc1N1CCC2(CC1)CC2 ; O=C(Nc1ccc(=O)n(C2CCOCC2)c1)c1ccc(NS(=O)(=O)CCO)cc1N1CCC2(CC1)CC2]

### Step 1

To a solution 5-nitropyridin-2(1H)-one (1 g, 7.138 mmol) in dioxane (5 mL) and 2-(3,6-dihydro-2H-pyran-4-yl)-4,4,5,5-tetramethyl-1,3,2-dioxaborolane (1.65 g, 7.852 mmol) were added Cu(OAc)₂ (0.26 g, 1.428 mmol) and TEA (3.969 mL, 28.551 mmol). The reaction mixture was stirred at 80° C. for 3 hrs under N₂ atmosphere. The reaction mixture was filtered through Celite and washed with MeOH. The filtrate was dried over anhydrous sodium sulfate, concentrated under reduced pressure, and purified by silica gel chromatography to afford EX06-1 (1.3 g). LCMS[M+H]⁺: 223.2.

### Step 2

To a mixture of EX06-1 (1.00 g, 4.50 mmol) and NH₄Cl (1.69 g, 31.5 mmol) in EtOH (10 mL) and H₂O (10 mL) was added iron powder (1.76 g, 31.5 mmol) in three portions. The mixture was stirred at 80° C. for 3 hrs. The mixture was filtered through Celite, and the filtrate was concentrated under reduced pressure. The obtained residue was diluted with water (100 mL) and extracted with EtOAc (50 mL×2). The organic layer was dried over anhydrous sodium sulfate, concentrated under reduced pressure, and purified by silica gel chromatography to afford EX06-2 (700 mg).

### Step 3

EX06-3 (90 mg) was prepared as described in Example 1 (EX01) Steps 5-6, except EX06-2 was used instead of EX01-4 in Step 5. LCMS[M+H]⁺: 532.3.

### Step 4

A solution of EX06-3 (100 mg, 2.36 mmol) and Pd/C (1.0 g, 10 wt %) in MeOH (10 mL) was degassed and flushed with H₂, and the reaction mixture stirred at 25° C. for 2 hrs under H₂ atmosphere. The reaction mixture was filtered, and the filtrate was concentrated under reduced pressure and purified by prep-HPLC to afford EX06 (20 mg). ¹H NMR (400 MHz, CDCl₃) δ 12.52 (s, 1H), 8.70 (d, J=2.7 Hz, 1H), 8.20 (d, J=8.5 Hz, 1H), 7.72 (s, 1H), 7.39 (d, J=2.1 Hz, 1H), 7.29 (dd, J=9.6, 2.8 Hz, 1H), 7.10 (dd, J=8.6, 2.1 Hz, 1H), 6.69 (d, J=9.5 Hz, 1H), 5.20 (tt, J=12.2, 4.4 Hz, 1H), 4.12 (dt, J=10.6, 4.6 Hz, 4H), 3.61 (t, J=11.3 Hz, 2H), 3.33 (t, J=5.1 Hz, 2H), 3.06 (t, J=5.3 Hz, 5H), 2.00 (qd, J=12.1, 4.5 Hz, 2H), 1.90 (dd, J=12.7, 3.9 Hz, 2H), 1.65-1.57 (m, 4H), 0.46 (s, 4H). LCMS [M+H]⁺: 531.3.

### Example 7: N-(8,8-difluoro-6,6a,7,8,9,10-hexahydrodipyrido[3,2-b:1′,2′-d][1,4]oxazin-2-yl)-4-((2-hydroxyethyl)sulfonamido)-2-(6-azaspiro[2.5]octan-6-yl)benzamide (EX07)

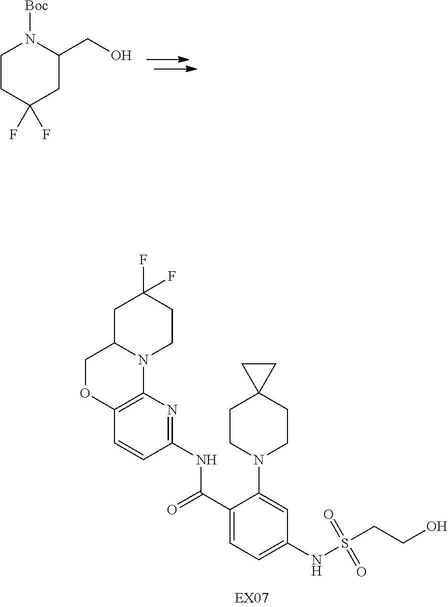 [SMILES: CC(C)(C)OC(=O)N1CCC(F)(F)CC1CO ; O=C(Nc1ccc2c(n1)N1CCC(F)(F)CC1CO2)c1ccc(NS(=O)(=O)CCO)cc1N1CCC2(CC1)CC2]

### Step 1

EX07 (3.3 mg) was prepared as described in Example 3 (EX03) Steps 1-6, except tert-butyl 4,4-difluoro-2-(hydroxymethyl)piperidine-1-carboxylate was used instead of tert-butyl 3-(hydroxymethyl) morpholine-4-carboxylate in Step 1. ¹H NMR (400 MHz, CD₃OD) δ 8.12 (d, J=8.5 Hz, 1H), 7.59 (d, J=8.4 Hz, 1H), 7.31 (s, 1H), 7.14 (d, J=8.7 Hz, 1H), 7.05 (d, J=8.4 Hz, 1H), 4.32 (dd, J=11.2, 2.9 Hz, 1H), 4.03 (dd, J=11.2, 6.2 Hz, 1H), 3.95 (t, J=6.2 Hz, 2H), 3.61-3.49 (m, 1H), 3.36 (t, J=6.3 Hz, 2H), 3.08 (t, J=5.4 Hz, 4H), 2.93 (t, J=12.7 Hz, 1H), 2.23-1.71 (m, 8H), 0.44 (s, 4H). LCMS [M+H]⁺: 578.4.

### Example 8: 4-((2-hydroxyethyl)sulfonamido)-N-(6-oxo-1-(4,4,4-trifluorobutyl)-1,6-dihydro pyridazin-3-yl)-2-(6-azaspiro[2.5]octan-6-yl)benzamide (EX08)

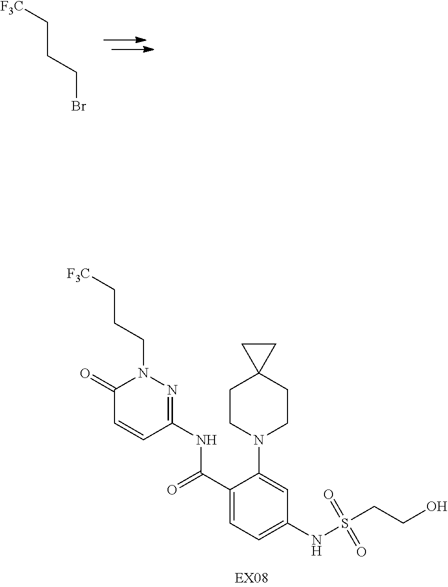 [SMILES: FC(F)(F)CCCBr ; O=C(Nc1ccc(=O)n(CCCC(F)(F)F)n1)c1ccc(NS(=O)(=O)CCO)cc1N1CCC2(CC1)CC2]

### Step 1

EX08 (50 mg) was prepared as described in Example 5 (EX05) Steps 1-4, except 4-bromo-1,1,1-trifluorobutane was used instead of 4-iodotetrahydro-2H-pyran in Step 1. ¹H NMR (400 MHz, CD₃OD) δ 8.53 (d, J=9.9 Hz, 1H), 8.13 (d, J=8.7 Hz, 1H), 7.35 (d, J=2.2 Hz, 1H), 7.18 (dd, J=8.7, 2.2 Hz, 1H), 7.09 (d, J=9.9 Hz, 1H), 4.24 (t, J=6.9 Hz, 2H), 3.96 (t, J=6.3 Hz, 2H), 3.38 (t, J=6.3 Hz, 3H), 3.10 (t, J=5.4 Hz, 5H), 2.39-2.24 (m, 2H), 2.21-2.08 (m, 2H), 1.92-1.64 (m, 4H), 0.46 (s, 4H). LCMS [M+H]⁺: 558.4.

### Example 9: N-(1-(4,4-difluorocyclohexyl)-2-oxo-1,2-dihydropyridin-3-yl)-4-((2-hydroxyethyl) sulfonamido)-2-(6-azaspiro[2.5]octan-6-yl)benzamide (EX09)

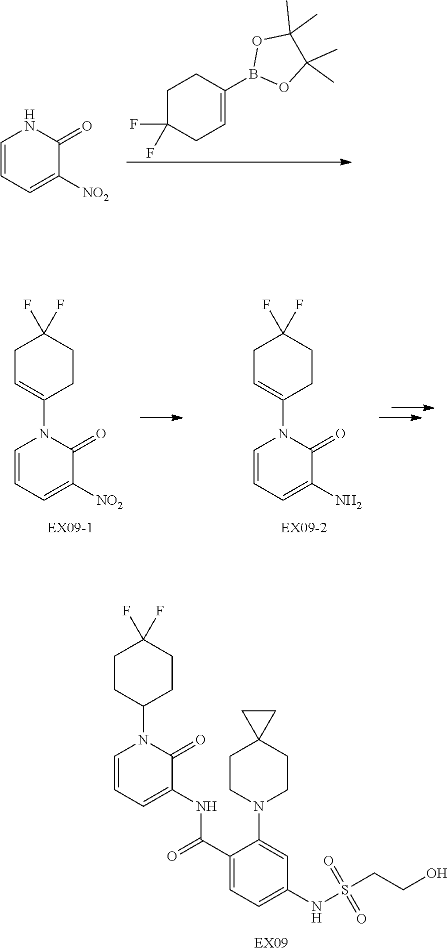

### Step 1

To a solution 3-nitropyridin-2(1H)-one (1 g, 7.13 mmol) in dioxane (5 mL) were added TEA (3.96 mL, 28.5 mmol), 2-(4,4-difluorocyclohex-1-en-1-yl)-4,4,5,5-tetramethyl-1,3,2-dioxaborolane (1.65 g, 7.85 mmol) and Cu(OAc)₂ (0.26 g, 1.42 mmol). The reaction mixture was stirred at 80 TC for 3 hrs. The reaction mixture was filtered through Celite and washed with MeOH. The filtrate was dried over anhydrous sodium sulfate, concentrated under reduced pressure, and purified by silica gel chromatography to afford EX09-1 (1.3 g). LCMS[M+H]⁺: 257.2.

### Step 2

A mixture of EX09-1 (1.00 g, 4.50 mmol) and Pd/C (10 wt %, 1.69 g, 31.5 mmol) in EtOH (10 mL) was stirred at 25° C. for 3 hrs under H₂ atmosphere. The reaction mixture was filtered through Celite, and the filtrate was concentrated under reduced pressure. The obtained residue was diluted with water (100 mL) and extracted with EtOAc (50 mL×2). The combined organic layer was dried over anhydrous sodium sulfate, concentrated under reduced pressure, and purified by prep-HPLC to afford EX09-2 (700 mg). LCMS[M+H]⁺: 229.1.

### Step 3

EX09 (20 mg) was prepared as described in Example 1 (EX01) Steps 5-6, except EX09-2 was used instead of EX01-4 in Step 5. ¹H NMR (400 MHz, CD₃OD) δ 8.60 (dd, J=7.4, 1.7 Hz, 1H), 7.99 (d, J=8.6 Hz, 1H), 7.42 (dd, J=7.1, 1.8 Hz, 1H), 7.27 (d, J=2.2 Hz, 1H), 7.09 (dd, J=8.6, 2.1 Hz, 1H), 6.45 (t, J=7.3 Hz, 1H), 5.06 (p, J=7.0, 6.5 Hz, 1H), 3.96 (t, J=6.3 Hz, 2H), 3.38 (t, J=6.3 Hz, 2H), 3.06 (t, J=5.4 Hz, 4H), 2.32-1.91 (m, 8H), 1.86-1.53 (m, 4H), 0.38 (s, 4H). LCMS [M+H]⁺: 565.5.

### Example 10: N-(8,8-difluoro-6,6a,7,8,9,10-hexahydrodipyrido[3,2-b:1′,2′-d][1,4]oxazin-4-yl)-4-((2-hydroxyethyl)sulfonamido)-2-(6-azaspiro[2.5]octan-6-yl)benzamide (EX10)

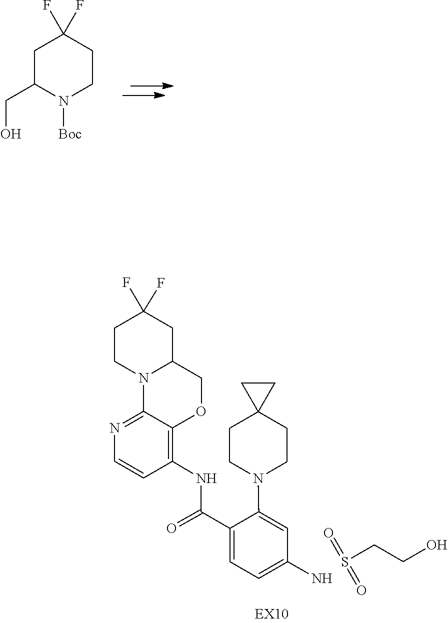

### Step 1

EX10 (20 mg) was prepared as described in Example 1 (EX01) Steps 1-6, except tert-butyl 4,4-difluoro-2-(hydroxymethyl)piperidine-1-carboxylate was used instead of tert-butyl 3-(hydroxymethyl) morpholine-4-carboxylate in Step 1. ¹H NMR (400 MHz, CD₃OD) δ 8.05 (d, J=8.6 Hz, 1H), 8.01 (d, J=5.7 Hz, 1H), 7.74 (d, J=5.7 Hz, 1H), 7.29 (d, J=2.1 Hz, 1H), 7.12 (dd, J=8.6, 2.1 Hz, 1H), 4.77 (d, J=13.7 Hz, 1H), 4.50 (dd, J=11.0, 2.8 Hz, 1H), 4.25 (dd, J=11.1, 5.4 Hz, 1H), 3.96 (t, J=6.2 Hz, 2H), 3.67 (d, J=12.0 Hz, 1H), 3.38 (t, J=6.2 Hz, 2H), 3.12-2.94 (m, 5H), 2.31-2.09 (m, 2H), 2.06-1.85 (m, 2H), 1.82-1.50 (m, 4H), 0.44 (s, 4H). LCMS [M+H]⁺: 578.4.

### Example 11: N-(1-(4,4-difluorocyclohexyl)-6-oxo-1,6-dihydropyridazin-3-yl)-4-((2-hydroxy ethyl)sulfonamido)-2-(6-azaspiro[2.5]octan-6-yl)benzamide (EX11)

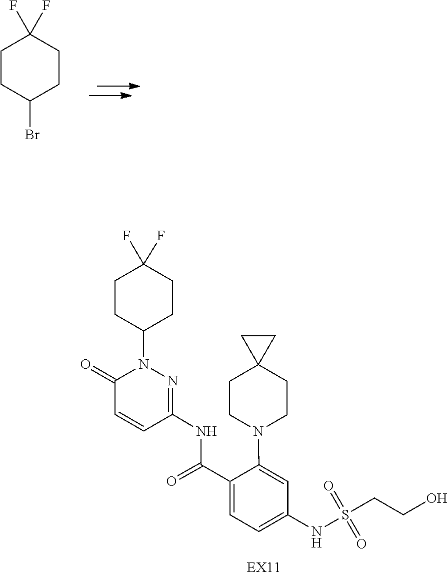 [SMILES: FC1(F)CCC(Br)CC1 ; O=C(Nc1ccc(=O)n(C2CCC(F)(F)CC2)n1)c1ccc(NS(=O)(=O)CCO)cc1N1CCC2(CC1)CC2]

### Step 1

EX11 (30 mg) was prepared as described in Example 5 (EX05) Steps 1-4, except 4-bromo-1,1-difluorocyclohexane was used instead of 4-iodotetrahydro-2H-pyran in Step 1. ¹H NMR (400 MHz, CD₃OD) δ 8.56 (d, J=9.9 Hz, 1H), 8.16 (d, J=8.6 Hz, 1H), 7.37 (d, J=2.2 Hz, 1H), 7.20 (dd, J=8.7, 2.2 Hz, 1H), 7.09 (d, J=9.9 Hz, 1H), 5.14-5.03 (m, 1H), 3.97 (t, J=6.2 Hz, 2H), 3.39 (t, J=6.2 Hz, 2H), 3.10 (t, J=5.3 Hz, 4H), 2.28-2.17 (m, 4H), 2.18-1.70 (m, 8H), 0.48 (s, 4H). LCMS [M+H]⁺: 566.5.

### Example 12: N-(1-(3,3-difluorocyclobutyl)-6-oxo-1,6-dihydropyridazin-3-yl)-4-((2-hydroxyethyl)sulfonamido)-2-(6-azaspiro[2.5]octan-6-yl)benzamide (EX12)

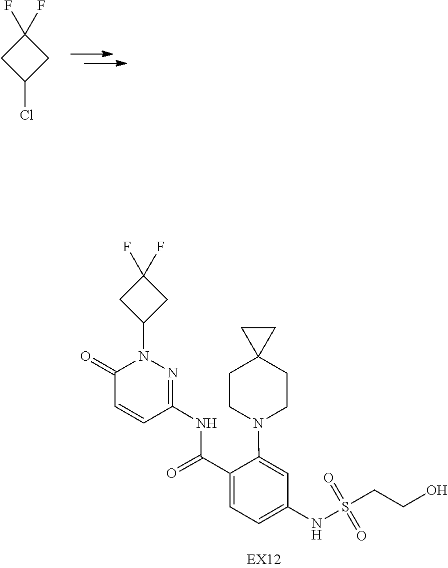 [SMILES: FC1(F)CC(Cl)C1 ; O=C(Nc1ccc(=O)n(C2CC(F)(F)C2)n1)c1ccc(NS(=O)(=O)CCO)cc1N1CCC2(CC1)CC2]

### Step 1

EX12 (10 mg) was prepared as described in Example 5 (EX05) Steps 1-4, except 3-chloro-1,1-difluorocyclobutane was used instead of 4-iodotetrahydro-2H-pyran in Step 1. ¹H NMR (400 MHz, DMSO-d₆) δ 13.44 (s, 1H), 10.25 (brs, 1H), 8.40 (d, J=10.0 Hz, 1H), 8.04 (d, J=8.6 Hz, 1H), 7.29 (d, J=2.1 Hz, 1H), 7.14 (dd, J=8.6, 2.1 Hz, 1H), 7.07 (d, J=10.0 Hz, 1H), 5.39-5.25 (m, 1H), 4.95 (brs, 1H), 3.76 (t, J=6.5 Hz, 2H), 3.40-3.35 (m, 2H), 3.12-2.93 (m, 8H), 1.68 (brs, 4H), 0.38 (s, 4H). LCMS [M+H]⁺: 538.3.

### Example 13: 1-((2-methoxyethyl)sulfonyl)-N-(6-morpholinopyridin-2-yl)-6-(6-azaspiro[2.5]octan-6-yl)indoline-5-carboxamide (EX13)

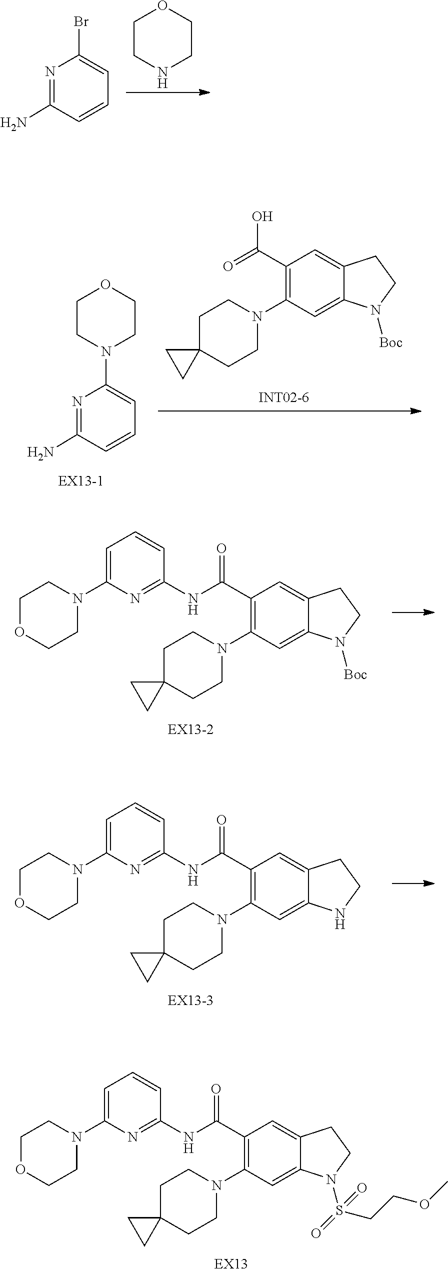

### Step 1

A mixture of morpholine (5.04 mL, 76.6 mmol) and 6-bromopyridin-2-amine (2.50 g, 14.5 mmol) was stirred at 150° C. for 2 hrs under microwave irritation. The mixture was concentrated under reduced pressure and purified by pre-TLC to afford EX13-1 (2.3 g). LCMS [M+H]⁺: 180.2.

### Step 2

To a solution of INT02-6 (550 mg, 1.48 mmol) in DCM (5 mL) were added DMF (0.12 mL, 1.48 mmol) and (COCl)₂ (0.15 mL, 1.77 mmol) at 0° C. The mixture was stirred at 25° C. for 1 hr. The mixture solution was slowly added to a solution of EX13-1 (266 mg, 1.48 mmol) and TEA (448 mg, 4.430 mmol) in DCM (5 mL) at 0° C. The mixture was stirred at 25° C. for 2 hrs. The reaction mixture was diluted with water (5 mL) and extracted with EtOAc (15 mL×3). The combined organic layer was washed with brine (8 mL), dried over anhydrous sodium sulfate, and concentrated under reduced pressure. The obtained residue was purified by silica gel chromatography to afford EX13-2 (250 mg). LCMS[M+H]⁺: 534.4.

### Step 3

To a solution of EX13-2 (200 mg, 0.38 mmol) in MeOH (1 mL) was added HCl (1 M in MeOH, 0.38 mL, 0.38 mmol). The mixture was stirred at 25° C. for 4 hrs. The reaction mixture was concentrated under reduced pressure to afford EX13-3 (115 mg). LCMS[M+H]⁺: 434.4.

### Step 4

To a solution of EX13-3 (100 mg, 0.23 mmol) in DCM (5 mL) were added 2-methoxyethane-1-sulfonyl chloride (73 mg, 0.46 mmol) and DIPEA (0.23 mL, 1.38 mmol). The mixture was stirred at 25° C. for 3 hrs. The reaction mixture was diluted with water (10 mL) and extracted with DCM (15 mL×3). The combined organic layer was washed with brine (8 mL×2), dried over anhydrous sodium sulfate, concentrated under reduced pressure, and purified by prep-HPLC to afford EX13 (150 mg). ¹H NMR (400 MHz, CD₃OD) δ 8.04 (s, 1H), 7.78 (t, J=8.2 Hz, 1H), 7.53 (s, 1H), 7.22 (s, 1H), 6.75 (d, J=8.6 Hz, 1H), 4.13 (t, J=8.6 Hz, 2H), 3.84 (t, J=4.9 Hz, 4H), 3.74 (t, J=5.9 Hz, 2H), 3.64-3.49 (m, 6H), 3.20 (t, J=8.6 Hz, 2H), 3.11 (s, 3H), 2.66 (s, 4H), 1.99-1.45 (m, 4H), 0.50 (s, 4H). LCMS [M+H]⁺: 556.4.

### Example 14: 1-((2-hydroxyethyl)sulfonyl)-N-(6-morpholinopyridin-2-yl)-6-(6-azaspiro[2.5]octan-6-yl)indoline-5-carboxamide (EX14)

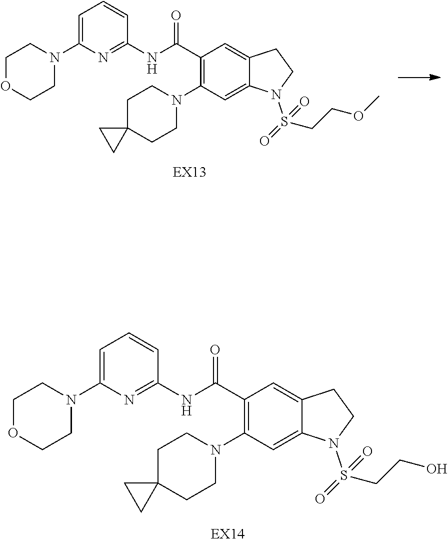 [SMILES: COCCS(=O)(=O)N1CCc2cc(C(=O)Nc3cccc(N4CCOCC4)n3)c(N3CCC4(CC3)CC4)cc21 ; O=C(Nc1cccc(N2CCOCC2)n1)c1cc2c(cc1N1CCC3(CC1)CC3)N(S(=O)(=O)CCO)CC2]

### Step 1

To a solution of EX13 (100 mg, 0.18 mmol) in DCM (5 mL) was dropwise added BBr₃ (45 mg, 0.18 mmol) at −45° C. under N₂ atmosphere. The mixture was stirred at −45° C. for 3 hrs under N₂ atmosphere. The reaction mixture was quenched with ice water (10 mL) and extracted with EtOAc (8 mL×3). The combined organic layer was washed with brine (10 mL×2), dried over anhydrous sodium sulfate, and concentrated under reduced pressure. The obtained residue was purified by prep-HPLC to afford EX-14 (9.5 mg). ¹H NMR (400 MHz, CD₃OD) δ 8.13 (s, 1H), 7.96 (t, J=8.4 Hz, 1H), 7.68 (s, 1H), 7.16 (s, 1H), 6.96 (d, J=8.9 Hz, 1H), 4.23 (t, J=8.7 Hz, 2H), 3.98 (t, J=5.5 Hz, 2H), 3.92-3.85 (m, 4H), 3.71-3.67 (m, 4H), 3.67-3.55 (m, 4H), 3.53 (t, J=5.5 Hz, 2H), 3.29-3.25 (m, 2H), 2.36-1.43 (m, 4H), 0.59 (s, 4H). LCMS [M+H]⁺: 542.3.

### Example 15: N-(2-(4,4-difluoropiperidin-1-yl)-6-methylpyrimidin-4-yl)-1-((2-hydroxyethyl) sulfonyl)-6-(6-azaspiro[2.5]octan-6-yl)indoline-5-carboxamide (EX15)

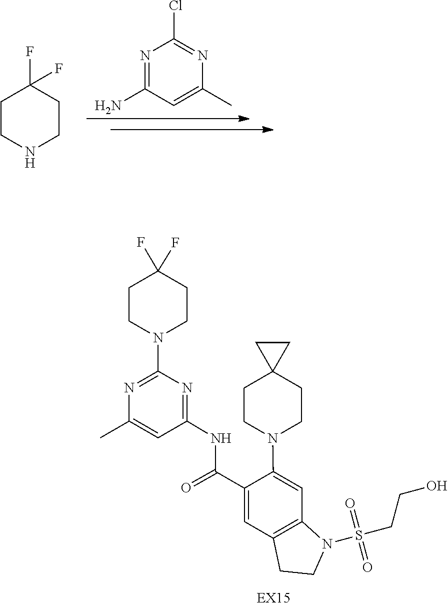

### Step 1

EX15 (8 mg) was prepared as described in Example 13 (EX13) Steps 1-4 and Example 14 (EX14) Step 1, except 4,4-difluoropiperidine and 2-chloro-6-methylpyrimidin-4-amine were used instead of morpholine and 6-bromopyridin-2-amine in Example 13 Step 1. ¹H NMR (400 MHz, DMSO-d₆) δ 13.69 (s, 1H), 7.98 (s, 1H), 7.41 (s, 1H), 7.29 (s, 1H), 4.08 (t, J=8.5 Hz, 2H), 3.92 (t, J=5.8 Hz, 4H), 3.78 (t, J=5.9 Hz, 2H), 3.46 (t, J=5.9 Hz, 2H), 3.15 (t, J=8.6 Hz, 2H), 2.98 (t, J=5.3 Hz, 4H), 2.32 (s, 3H), 2.06-1.93 (m, 4H), 1.76 (brs, 4H), 0.40 (s, 4H). LCMS [M+H]⁺: 591.5.

### Example 16: 1-((2-hydroxyethyl)sulfonyl)-N-(6-oxo-1-(4,4,4-trifluorobutyl)-1,6-dihydro pyridazin-3-yl)-6-(6-azaspiro[2.5]octan-6-yl)indoline-5-carboxamide (EX16)

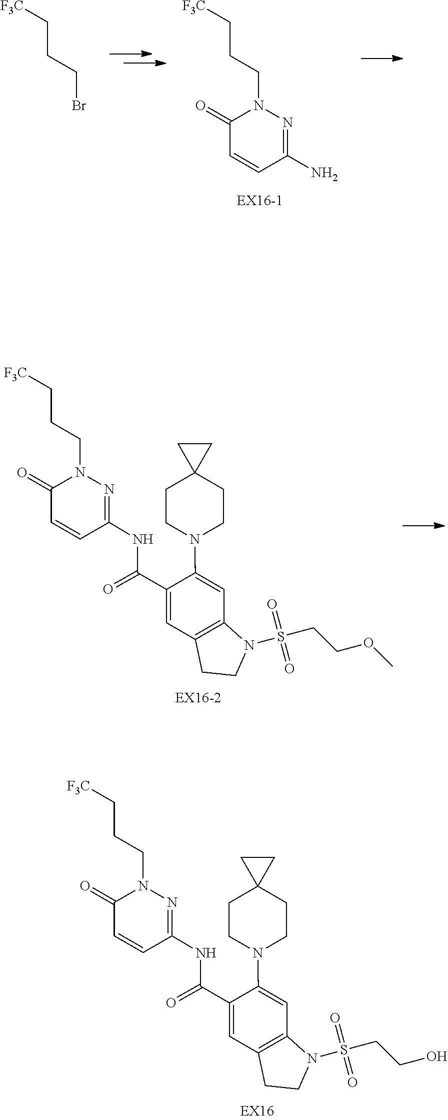 [SMILES: FC(F)(F)CCCBr ; Nc1ccc(=O)n(CCCC(F)(F)F)n1 ; COCCS(=O)(=O)N1CCc2cc(C(=O)Nc3ccc(=O)n(CCCC(F)(F)F)n3)c(N3CCC4(CC3)CC4)cc21 ; O=C(Nc1ccc(=O)n(CCCC(F)(F)F)n1)c1cc2c(cc1N1CCC3(CC1)CC3)N(S(=O)(=O)CCO)CC2]

### Step 1

EX16-1 (920 mg) was prepared as described in Example 5 (EX05) Steps 1-3, except 4-bromo-1,1,1-trifluorobutane was used instead of 4-iodotetrahydro-2H-pyran in Step 1. LCMS[M+H]⁺: 222.1.

### Step 2

A mixture of EX16-1 (15 mg, 0.067 mmol), INT02 (22 mg, 6.47 mmol) and pyridine (0.045 mL, 0.558 mmol) in DCM (1 mL) was stirred at 25° C. for 5 hrs under N₂ atmosphere. The reaction mixture was filtered, and the filtrate was diluted with water (15 mL) and extracted with EtOAc (30 mL×3). The combined organic layer was washed with brine (5 mL×2), dried over anhydrous sodium sulfate, and concentrated under reduced pressure to afford EX16-2 (15 mg). LCMS[M+H]⁺: 598.4.

### Step 3

EX16 (6 mg) was prepared as described in Example 14 (EX14) except EX16-2 was used instead of EX13. ¹H NMR (400 MHz, CD₃OD) δ 8.53 (d, J=9.9 Hz, 1H), 8.05 (s, 1H), 7.47 (s, 1H), 7.09 (d, J=9.9 Hz, 1H), 4.24 (t, J=7.0 Hz, 2H), 4.17 (t, J=8.6 Hz, 2H), 3.97 (t, J=5.8 Hz, 2H), 3.43 (t, J=5.8 Hz, 2H), 3.21 (t, J=8.6 Hz, 2H), 3.09 (t, J=5.4 Hz, 4H), 2.38-2.24 (m, 2H), 2.19-2.09 (m, 2H), 1.78 (brs, 4H), 0.47 (s, 4H). LCMS [M+H]⁺: 584.5.

### Example 17: 1-((2-hydroxyethyl)sulfonyl)-N-(6-oxo-1-(3,3,3-trifluoropropyl)-1,6-dihydro pyridazin-3-yl)-6-(6-azaspiro[2.5]octan-6-yl)indoline-5-carboxamide (EX17)

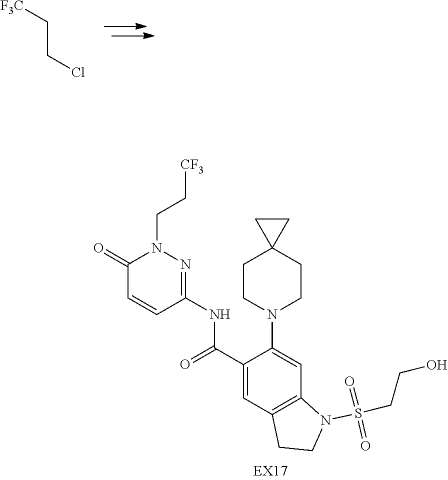 [SMILES: FC(F)(F)CCCl ; O=C(Nc1ccc(=O)n(CCC(F)(F)F)n1)c1cc2c(cc1N1CCC3(CC1)CC3)N(S(=O)(=O)CCO)CC2]

### Step 1

EX17 (25 mg) was prepared as described in Example 16 (EX16) Steps 1-3, except 3-chloro-1,1,1-trifluoropropane was used instead of 4-bromo-1,1,1-trifluorobutane in Step 1. ¹H NMR (400 MHz, DMSO-d₆) δ 13.82 (s, 1H), 8.35 (d, J=10.0 Hz, 1H), 7.95 (s, 1H), 7.29 (s, 1H), 7.09 (d, J=10.0 Hz, 1H), 5.04 (t, J=5.2 Hz, 1H), 4.27 (t, J=6.8 Hz, 2H), 4.07 (t, J=8.6 Hz, 2H), 3.77 (q, J=5.7 Hz, 2H), 3.46 (t, J=5.9 Hz, 2H), 3.13 (t, J=8.5 Hz, 2H), 2.98 (t, J=5.3 Hz, 4H), 2.81 (dtd, J=17.9, 11.4, 6.6 Hz, 2H), 1.66 (s, 4H), 0.39 (s, 4H). LCMS [M+H]⁺: 570.3.

### Example 18: N-(1-(3,3-difluorocyclobutyl)-6-oxo-1,6-dihydropyridazin-3-yl)-1-((2-hydroxyethyl)sulfonyl)-6-(6-azaspiro[2.5]octan-6-yl)indoline-5-carboxamide (EX18)

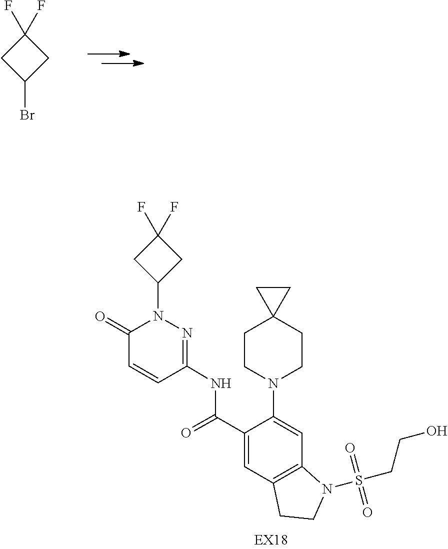 [SMILES: FC1(F)CC(Br)C1 ; O=C(Nc1ccc(=O)n(C2CC(F)(F)C2)n1)c1cc2c(cc1N1CCC3(CC1)CC3)N(S(=O)(=O)CCO)CC2]

### Step 1

EX18 (3.4 mg) was prepared as described in Example 16 (EX16) Steps 1-3, except 3-bromo-1,1-difluorocyclobutane was used instead of 4-bromo-1,1,1-trifluorobutane in Step 1. ¹H NMR (400 MHz, DMSO-d₆) δ 13.79 (s, 1H), 8.41 (d, J=10.0 Hz, 1H), 7.97 (s, 1H), 7.30 (s, 1H), 7.07 (d, J=10.0 Hz, 1H), 5.30 (tt, J=12.9, 6.9 Hz, 1H), 4.08 (t, J=8.6 Hz, 2H), 3.77 (t, J=5.9 Hz, 2H), 3.46 (t, J=5.9 Hz, 3H), 3.14 (t, J=8.6 Hz, 2H), 3.11-2.95 (m, 8H), 1.91-1.47 (m, 4H), 0.40 (s, 4H). LCMS [M+H]⁺: 564.2.

### Example 19: N-(8,8-difluoro-6,6a,7,8,9,10-hexahydrodipyrido[3,2-b:1′,2′-d][1,4]oxazin-4-yl)-1-((2-hydroxyethyl)sulfonyl)-6-(6-azaspiro[2.5]octan-6-yl)indoline-5-carboxamide (EX19)

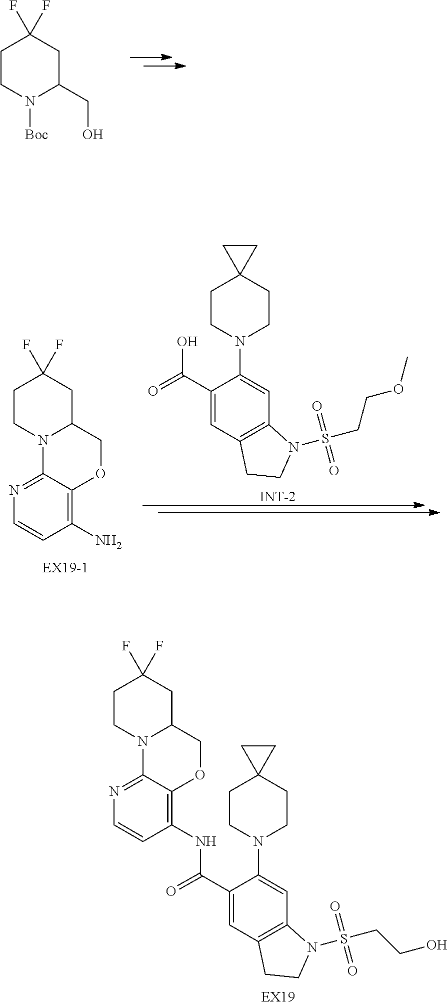

### Step 1

EX19-1 (11 mg) was prepared as described in Example 1 (EX01) Steps 1-4, except tert-butyl 4,4-difluoro-2-(hydroxymethyl)piperidine-1-carboxylate was used instead of tert-butyl 3-(hydroxymethyl) morpholine-4-carboxylate in Step 1. LCMS [M+H]⁺: 242.1.

### Step 2

EX19 (3 mg) was prepared as described in Example 16 (EX16) Steps 2-3, except EX19-1 was used instead of EX16-1 in Step 2. ¹H NMR (400 MHz, CD₃OD) δ 8.00 (d, J=5.8 Hz, 1H), 7.97 (s, 1H), 7.74 (d, J=5.8 Hz, 1H), 7.41 (s, 1H), 4.77 (d, J=14.0 Hz, 1H), 4.50 (dd, J=11.3, 2.6 Hz, 1H), 4.24 (dd, J=11.1, 5.4 Hz, 1H), 4.16 (t, J=8.6 Hz, 2H), 3.97 (t, J=5.8 Hz, 2H), 3.67 (d, J=12.2 Hz, 1H), 3.42 (t, J=5.9 Hz, 2H), 3.19 (t, J=8.6 Hz, 2H), 3.11-2.93 (m, 5H), 2.18 (dt, J=39.3, 12.2 Hz, 2H), 2.06-1.85 (m, 2H), 1.81-1.48 (m, 4H), 0.44 (s, 4H). LCMS [M+H]⁺: 604.4.

### Example 20: N-(2-(4,4-difluoropiperidin-1-yl)-6-methylpyrimidin-4-yl)-1-(methylsulfonyl)-6-(6-azaspiro[2.5]octan-6-yl)indoline-5-carboxamide (EX20)

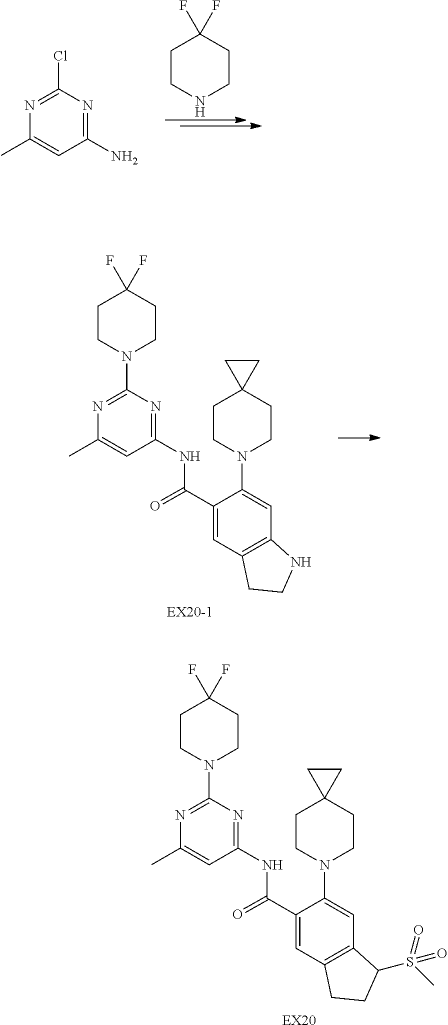

### Step 1

EX20-1 (90 mg) was prepared as described in Example 13 (EX13) Steps 1-3, except 4,4-difluoropiperidine and 2-chloro-6-methylpyrimidin-4-amine were used instead of morpholine and 6-bromopyridin-2-amine in Step 1. LCMS [M+H]⁺: 483.2.

### Step 2

EX20 (23 mg) was prepared as described in Example 13 (EX13) Step 4, except EX20-1 and methanesulfonyl chloride were used instead of EX13-3 and 2-methoxyethane-1-sulfonyl chloride. ¹H NMR (400 MHz, CD₃OD) δ 8.07 (s, 1H), 7.45 (s, 1H), 7.44 (s, 1H), 4.07 (t, J=8.5 Hz, 2H), 3.99 (t, J=5.9 Hz, 4H), 3.20 (t, J=8.4 Hz, 4H), 3.06 (t, J=5.3 Hz, 4H), 3.02 (s, 3H), 2.36 (s, 3H), 2.05-1.89 (m, 6H), 0.44 (s, 4H). LCMS [M+H]⁺: 561.2.

### Example 21: N-(1-(4,4-difluorocyclohexyl)-6-oxo-1,6-dihydropyridazin-3-yl)-1-((2-hydroxyethyl)sulfonyl)-6-(6-azaspiro[2.5]octan-6-yl)indoline-5-carboxamide (EX21)

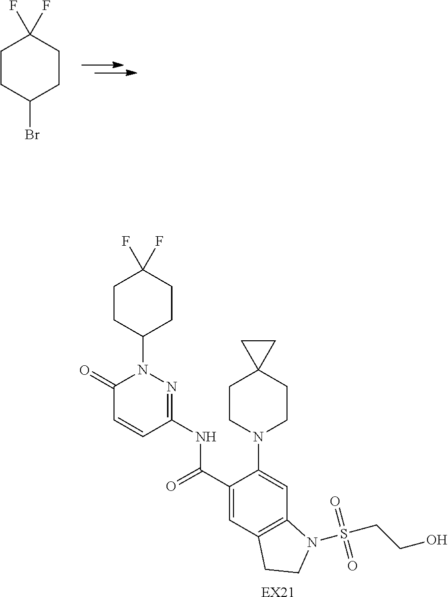

### Step 1

EX21 (6 mg) was prepared as described in Example 16 (EX16) Steps 1-3, except 4-bromo-1,1-difluorocyclohexane was used instead of 4-bromo-1,1,1-trifluorobutane in Step 1. ¹H NMR (400 MHz, DMSO-d₆) δ 13.93 (s, 1H), 8.38 (d, J=10.0 Hz, 1H), 7.97 (s, 1H), 7.30 (s, 1H), 7.07 (d, J=9.9 Hz, 1H), 5.14-4.92 (m, 2H), 4.08 (t, J=8.7 Hz, 2H), 3.78 (q, J=5.7 Hz, 2H), 3.50-3.44 (m, 2H), 3.14 (t, J=8.5 Hz, 2H), 2.98 (brs, 4H), 2.24-1.57 (m, 12H), 0.40 (s, 4H). LCMS [M+H]⁺: 592.4.

### Example 22: 1-((2-hydroxyethyl)sulfonyl)-N-(1-isopropyl-6-oxo-1,6-dihydropyridazin-3-yl)-6-(6-azaspiro[2.5]octan-6-yl)indoline-5-carboxamide (EX22)

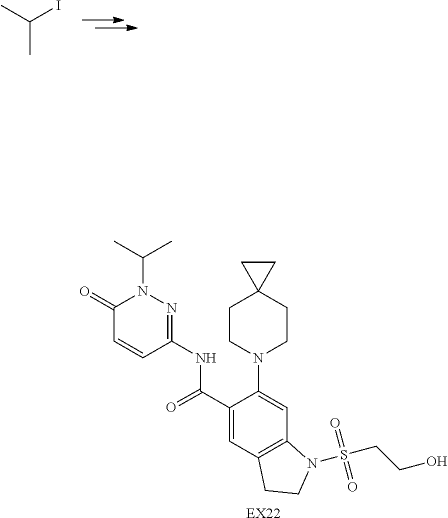

### Step 1

EX22 (3 mg) was prepared as described in Example 16 (EX16) Steps 1-3, except 2-iodopropane was used instead of 4-bromo-1,1,1-trifluorobutane in Step 1. ¹H NMR (400 MHz, DMSO-d₆) δ 13.90 (s, 1H), 8.35 (d, J=9.9 Hz, 1H), 7.97 (s, 1H), 7.29 (s, 1H), 7.02 (d, J=9.9 Hz, 1H), 5.17 (p, J=6.6 Hz, 1H), 5.06 (t, J=5.1 Hz, 1H), 4.10-4.07 (m, 2H), 3.80-3.74 (m, 2H), 3.45 (t, J=6.0 Hz, 2H), 3.13 (t, J=8.2 Hz, 3H), 2.99 (t, J=5.3 Hz, 4H), 1.95-1.49 (m, 4H), 1.30 (d, J=6.6 Hz, 6H), 0.40 (s, 4H). LCMS [M+H]⁺: 516.4.

### Example 23: N-(2-(3,3-difluoropyrrolidin-1-yl)-6-methylpyrimidin-4-yl)-1-((2-hydroxyethyl)sulfonyl)-6-(6-azaspiro[2.5]octan-6-yl)indoline-5-carboxamide (EX23)

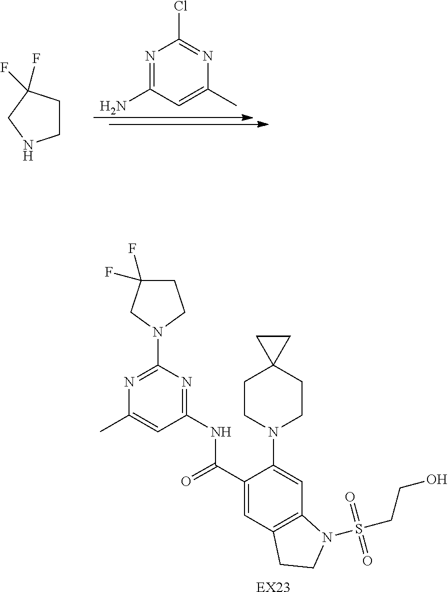

### Step 1

EX23 (1.8 mg) was prepared as described in Example 13 (EX13) Steps 1-4 and Example 14 (EX14), except 3,3-difluoropyrrolidine and 2-chloro-6-methylpyrimidin-4-amine were used instead of morpholine and 6-bromopyridin-2-amine in Example 13 Step 1. ¹H NMR (400 MHz, DMSO-d₆) δ 13.87 (s, 1H), 7.98 (s, 1H), 7.42 (s, 1H), 7.29 (s, 1H), 5.05 (t, J=5.2 Hz, 1H), 4.08 (t, J=8.6 Hz, 2H), 3.91 (t, J=13.2 Hz, 2H), 3.81-3.69 (m, 4H), 3.47 (t, J=5.9 Hz, 2H), 3.15 (t, J=8.5 Hz, 2H), 3.02-2.93 (m, 4H), 2.63-2.54 (m, 2H), 2.33 (brs, 4H), 1.77 (s, 2H), 0.39 (s, 4H). LCMS [M+H]⁺: 577.4.

### Example 24: N-(2-(3,3-difluoroazetidin-1-yl)-6-methylpyrimidin-4-yl)-1-((2-hydroxyethyl)sulfonyl)-6-(6-azaspiro[2.5]octan-6-yl)indoline-5-carboxamide (EX24)

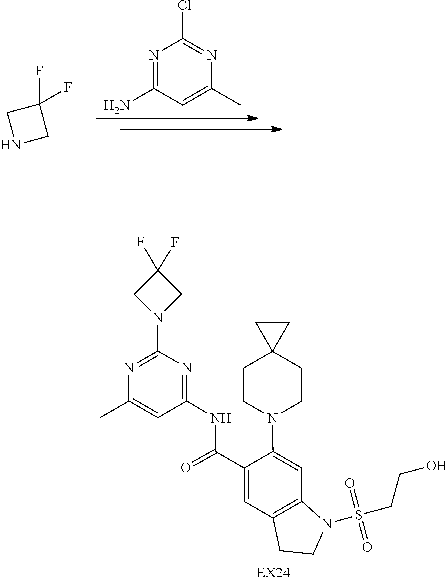

### Step 1

EX24 (28 mg) was prepared as described in Example 13 (EX13) Steps 1-4 and Example 14 (EX14), except 3,3-difluoroazetidine and 2-chloro-6-methylpyrimidin-4-amine were used instead of morpholine and 6-bromopyridin-2-amine in Example 13 Step 1. ¹H NMR (400 MHz, DMSO-d₆) δ 13.86 (s, 1H), 7.96 (s, 1H), 7.51 (s, 1H), 7.28 (s, 1H), 5.04 (t, J=5.3 Hz, 1H), 4.43 (t, J=12.5 Hz, 4H), 4.08 (t, J=8.6 Hz, 2H), 3.77 (q, J=5.6 Hz, 2H), 3.47 (d, J=5.9 Hz, 2H), 3.14 (t, J=8.5 Hz, 2H), 2.97 (t, J=5.3 Hz, 4H), 2.33 (s, 3H), 1.74 (brs, 4H), 0.40 (s, 4H). LCMS [M+H]⁺: 563.3.

### Example 25: N-(2-(4,4-difluoropiperidin-1-yl)-6-methylpyrimidin-4-yl)-1-(3-hydroxy propanoyl)-6-(6-azaspiro[2.5]octan-6-yl)indoline-5-carboxamide (EX25)

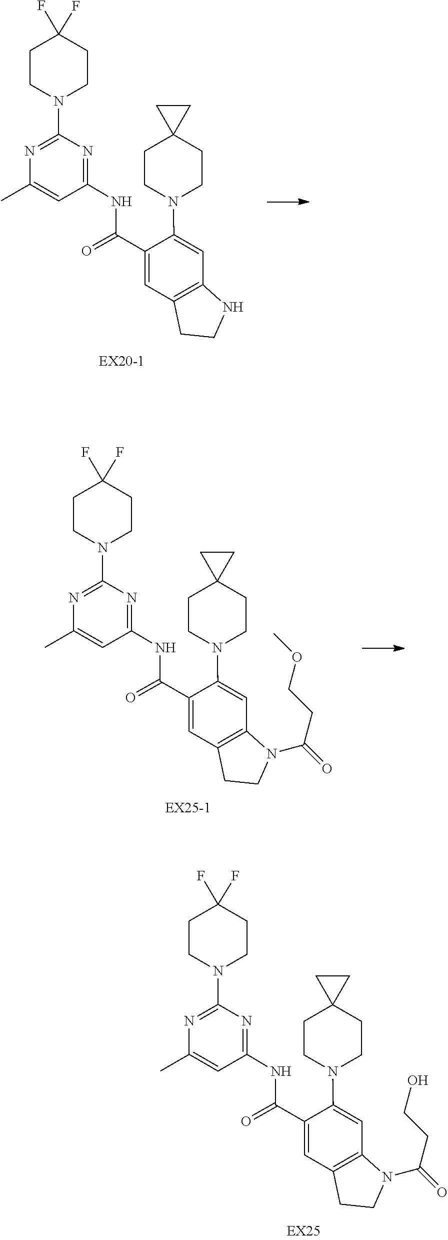 [SMILES: Cc1cc(NC(=O)c2cc3c(cc2N2CCC4(CC2)CC4)NCC3)nc(N2CCC(F)(F)CC2)n1 ; COCCC(=O)N1CCc2cc(C(=O)Nc3cc(C)nc(N4CCC(F)(F)CC4)n3)c(N3CCC4(CC3)CC4)cc21 ; Cc1cc(NC(=O)c2cc3c(cc2N2CCC4(CC2)CC4)N(C(=O)CCO)CC3)nc(N2CCC(F)(F)CC2)n1]

### Step 1

To a solution of EX20-1 (100 mg, 0.21 mmol) in DCM (5 mL) were added DIEA (0.21 mL, 1.24 mmol) and 3-methoxypropanoyl chloride (30 mg, 0.25 mmol). The mixture was stirred at 25° C. for 2 hrs. The reaction mixture was diluted with water (10 mL) and extracted with DCM (8 mL×3). The combined organic layer was washed with brine (15 mL×2), dried over anhydrous sodium sulfate, concentrated under reduced pressure to afford EX25-1 (150 mg). LCMS[M+H]⁺: 569.5.

### Step 2

EX25 (20 mg) was prepared as described in Example 14 (EX14), except EX25-1 was used instead of EX13. ¹H NMR (400 MHz, DMSO-d₆) δ 13.92 (s, 1H), 8.23 (s, 1H), 7.97 (s, 1H), 7.40 (s, 1H), 4.65 (d, J=5.5 Hz, 1H), 4.19 (t, J=8.5 Hz, 2H), 3.91 (t, J=5.5 Hz, 4H), 3.74 (q, J=5.6, 5.2 Hz, 2H), 3.17 (t, J=8.5 Hz, 2H), 2.96 (t, J=5.3 Hz, 4H), 2.65 (t, J=6.5 Hz, 2H), 2.31 (s, 3H), 1.98 (tt, J=13.6, 5.6 Hz, 4H), 1.75 (brs, 4H), 0.39 (s, 4H). LCMS [M+H]⁺: 555.5.

### Example 26: KIF18A Assay

The KIF18A assay was performed as follows:

1.1. Prepared 1× kinase Buffer
1.2. Compound screening:
a) Added 40 μL test compound to 384-well dilution plate
b) Diluted compound 1:3 in succession in DMSO for each column for 10 pts
c) Transferred 0.1 μL diluted compound solution to 384 assay plate using Echo, each column containing 2 replicates
d) Added 5 μL enzyme working solution to 384-well assay plate, centrifuge 1000 RPM for 1 min
e) Incubated at 25° C. for 15 min
f) Added 5 μL ATP working solution to initiate reaction
g) Incubated at 25° C. for 60 min
h) Added 10 μL ADP Glo reagent, centrifuge 1000 RPM for 1 min
i) Incubated at 25° C. for 60 min
j) Added 20 μL kinase detection reagent, centrifuge 1000 RPM for 1 min
k) Incubated at 25° C. for 60 min
l) Read Luminescence Signal on Envision 2104 plate reader.
   1.3. Data analysis−Calculated IC₅₀ and plot dose-response curve of compounds:
Calculated IC₅₀ by fitting % Inhibition values and log of compound concentrations to nonlinear regression (dose response−variable slope) with GraphPad 6.0.

$$Y = Bottom + ( Top - Bottom ) / ( 1 + 1 {0}^{⋀} ( ( Log {IC}_{50} - X ) * HillSlope ) )$$

X: log of inhibitor concentration; Y: % Inhibition

The data from Example 26 is shown in Table 1.

TABLE 1

| Compound | KIF18A IC₅₀ (nM) |
|---|---|
| EX01 | A |
| EX02 | A |
| EX03 | A |
| EX04 | B |
| EX05 | B |
| EX06 | C |
| EX07 | A |
| EX08 | A |
| EX09 | B |
| EX10 | A |
| EX11 | A |
| EX12 | A |
| EX13 | C |
| EX14 | A |
| EX15 | A |
| EX16 | A |
| EX17 | A |
| EX18 | A |
| EX19 | A |
| EX20 | B |
| EX21 | A |
| EX22 | A |
| EX23 | NT |
| EX24 | NT |
| EX25 | C |

A = IC₅₀ > 0 and ≤ 50 nM;
B = IC₅₀ > 50 nM and ≤ 500 nM;
C = IC₅₀ > 500 nM and ≤ 15 μM;
NT = not tested.

It is claimed:

1. A compound of Formula (I), or a pharmaceutically acceptable salt thereof: 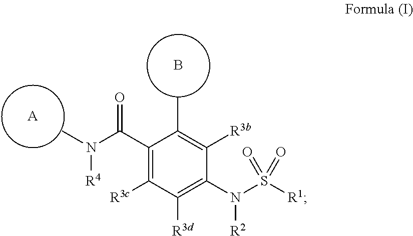
wherein:
R¹ is selected from C₁₋₆alkyl and C₃₋₆cycloalkyl, wherein C₁₋₆alkyl and C₃₋₆cycloalkyl are optionally substituted with one, two, or three groups selected from R^{9a};
R² is selected from hydrogen and C₁₋₆alkyl;
ring B is selected from C₃₋₁₂cycloalkyl, C₂₋₁₄heterocycloalkyl, C₆₋₁₀aryl, and C₁₋₉heteroaryl, wherein C₃₋₁₂cycloalkyl, C₂₋₁₄heterocycloalkyl, C₆₋₁₀aryl, and C₁₋₉heteroaryl are optionally substituted with one, two, or three groups selected from R^{9b};
R^{3b} and R^{3c} are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9c};
R^{3d} is selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9d}; or R² and R^{3d} are combined to form a C₂₋₉heterocycloalkyl or C₂₋₉-heteroaryl, wherein the C₂₋₉heterocycloalkyl and C₂₋₉heteroaryl are optionally substituted with one, two, or three groups selected from R^{9d};
R⁴ is selected from hydrogen and C₁₋₆alkyl;
ring A is a pyridinone ring, a pyridazinone ring, a bicyclic heterocyclic ring, or a tricyclic heterocyclic ring, wherein the pyridinone ring, pyridazinone ring, bicyclic heterocyclic ring, and tricyclic heterocyclic ring are optionally substituted with one or more R^{6a} groups;
each R^{6a} is independently selected from halogen, hydroxy, oxo, —CN, C₁₋₆alkyl, C₁₋₆haloalkyl, C₁₋₆alkoxy, C₂₋₆alkenyl, C₂₋₆alkynyl, C₃₋₆cycloalkyl, C₂₋₆heterocycloalkyl, C₆₋₁₀aryl, and C₁₋₉heteroaryl; wherein C₁₋₆alkyl, C₁₋₆haloalkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9e};
each R^{9a}, R^{9b}, R^{9e}, R^{9d}, and R^{9e} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₁₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂;
each R¹⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₆heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₁₀alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

2. The compound of claim 1, or a pharmaceutically acceptable salt thereof, wherein ring A is a pyridinone ring or a pyridazinone ring, wherein the pyridinone ring and pyridazinone ring are optionally substituted with one or more R^{6a} groups.

3. The compound of claim 1 or 2, or a pharmaceutically acceptable salt thereof, wherein each R^{6a} is independently selected from halogen, C₁₋₆alkyl, C₁₋₆haloalkyl, C₃₋₆cycloalkyl, and C₂₋₉heterocycloalkyl; wherein C₁₋₆alkyl, C₁₋₆haloalkyl, C₃₋₆cycloalkyl, and C₂₋₉heterocycloalkyl are optionally substituted with one, two, or three groups selected from R^{9e}.

4. The compound of claim 1, or a pharmaceutically acceptable salt thereof, wherein ring A is a bicyclic heterocyclic ring or a tricyclic heterocyclic ring, wherein the bicyclic heterocyclic ring and tricyclic heterocyclic ring are optionally substituted with one or more R^{6a} groups.

5. The compound of claim 1 or 4, or a pharmaceutically acceptable salt thereof, wherein ring A is a bicyclic heteroaryl ring or a tricyclic heteroaryl ring, wherein the bicyclic heteroaryl ring and tricyclic heteroaryl ring are optionally substituted with one or more R^{6a} groups.

6. The compound of claim 1 or 4, or a pharmaceutically acceptable salt thereof, wherein ring A is an 11-15 membered bicyclic heterocyclic ring or an 11-17 membered tricyclic heterocyclic ring, wherein the 11-15 membered bicyclic heterocyclic ring and 11-17 membered tricyclic heterocyclic ring are optionally substituted with one or more R^{6a} groups.

7. The compound of any one of claims 1 to 6, or a pharmaceutically acceptable salt thereof, wherein each R^{6a} is independently selected from halogen, C₁₋₆alkyl, C₁₋₆haloalkyl, C₃₋₆cycloalkyl, and C₂₋₉heterocycloalkyl; wherein C₁₋₆alkyl, C₁₋₆haloalkyl, C₃₋₆cycloalkyl, and C₂₋₉heterocycloalkyl are optionally substituted with one, two, or three groups selected from R^{9c}.

8. The compound of any one of claims 1-7, or a pharmaceutically acceptable salt thereof, wherein R² is hydrogen and R^{3d} is hydrogen.

9. The compound of any one of claims 1-7, or a pharmaceutically acceptable salt thereof, wherein R² and R^{3d} are combined to form a C₂₋₉heterocycloalkyl or C₂₋₉heteroaryl, wherein the C₂₋₉heterocycloalkyl and C₂₋₉heteroaryl are optionally substituted with one, two, or three groups selected from R^{9d}.

10. A compound of Formula (II), or a pharmaceutically acceptable salt thereof: 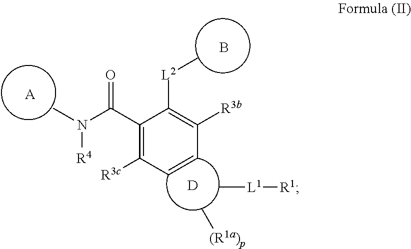
wherein:
ring A is selected from C₂₋₁₄heterocycloalkyl, C₂₋₁₄heteroaryl, and C₆₋₁₀aryl, wherein C₂₋₁₄heterocycloalkyl, C₂₋₁₄heteroaryl, and C₆₋₁₀aryl are optionally substituted with one or more R^{6a} groups;
ring D is selected from a 5- to 6-membered heterocycloalkyl ring, a 5- to 6-membered heteroaryl ring, and a 5- to 6-membered cycloalkyl ring;
L¹ is selected from —S(O)₂— and —C(O)—;
L² is selected from a bond, —O—, —S—, —S(O)—, —S(O)₂—, —C(R^{8a})(R^{8b})—, —OC(R^{8a})(R^{8b})—, —C(R^{8a})(R^{8b})O—, and —N(R^{8c})—;
R¹ is selected from C₁₋₆alkyl and C₃₋₆cycloalkyl, wherein C₁₋₆alkyl and C₃₋₆cycloalkyl are optionally substituted with one, two, or three groups selected from R^{9a};
each R^{1a} is independently selected from halogen, —CN, oxo, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9d};
ring B is selected from C₃₋₁₂cycloalkyl, C₂₋₁₄heterocycloalkyl, C₆₋₁₀aryl, and C₁₋₉heteroaryl, wherein C₃₋₁₂cycloalkyl, C₂₋₁₄heterocycloalkyl, C₆₋₁₀aryl, and C₁₋₉heteroaryl are optionally substituted with one, two, or three groups selected from R^{9b};
R^{3b} and R^{3c} are independently selected from hydrogen, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹¹, —SR¹⁰, —SF₅, —N(R¹⁰)(R¹¹), —C(O)OR¹⁰, —OC(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹¹)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₉alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9c};
R⁴ is selected from hydrogen and C₁₋₆alkyl;
each R^{6a} is independently selected from halogen, hydroxy, oxo, —CN, C₁₋₆alkyl, C₁₋₆haloalkyl, C₁₋₆alkoxy, C₂₋₆alkenyl, C₂₋₆alkynyl, C₃₋₆cycloalkyl, C₂₋₉heterocycloalkyl, C₆₋₁₀aryl, C₁₋₉heteroaryl, —C(O)OR¹⁰, —C(O)R¹³, —C(O)N(R¹⁰)(R¹¹), —S(O)₂R¹³, and —S(O)₂N(R¹⁰)(R¹¹)—; wherein C₁₋₆alkyl, C₁₋₆haloalkyl, C₁₋₆alkoxy, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9e};
R^{8a} and R^{8b} are independently selected from hydrogen, halogen, C₁₋₆alkyl, C₁₋₆haloalkyl, C₂₋₉alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from R^{9f};
R^{8c} is selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl; wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₆heteroaryl are optionally substituted with one, two, or three groups selected from R^{9f};
each R^{9a}, R^{9b}, R^{c}, R^{9d}, R^{9e}, and R^{9f} are each independently selected from halogen, oxo, —CN, C₁₋₆alkyl, C₂₋₆alkenyl, C₂₋₆alkynyl, C₃₋₁₀cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —CH₂—C₁₋₉heteroaryl, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹), —Si(C₁₋₆alkyl)₃, and —P(O)(R¹⁰)₂, wherein C₁₋₆alkyl, C₂₋₆alkenyl, C₂₋₆alkynyl, C₃₋₆cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, —CH₂—C₁₋₉heteroaryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁰, —SR¹⁰, —SF₅, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, —N═S(═O)(R¹³)₂, —S(═O)(═NH)N(R¹⁰)(R¹¹), —S(═O)(═NH)C(R¹⁰)(R¹¹), —S(═O)(═NR¹³)R¹³, —CH₂C(O)N(R¹⁰)(R¹¹), —CH₂N(R¹²)C(O)R¹³, —CH₂S(O)₂R¹³, —CH₂S(O)₂N(R¹⁰)(R¹¹) and —P(O)(R¹⁰)₂;
each R¹⁰ is independently selected from hydrogen, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₆heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆ haloalkyl, C₁₋₆alkoxy, C₃₋₆cycloalkyl, C₂₋₉heterocycloalkyl, C₆₋₁₀aryl, and C₁₋₆heteroaryl;
each R¹¹ is independently selected from hydrogen, C₁₋₆alkyl, and C₁₋₆haloalkyl; or R¹⁰ and R¹¹, together with the nitrogen to which they are attached, form a C₂₋₉heterocycloalkyl;
each R¹² is independently selected from hydrogen, C₁₋₆alkyl, and C₁₋₆haloalkyl;
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, —CN, hydroxy, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl; and
p is 0, 1, 2, or 3.

11. The compound of claim 10, or a pharmaceutically acceptable salt thereof, wherein ring D is a 5- to 6-membered heterocycloalkyl ring.

12. The compound of claim 10, or a pharmaceutically acceptable salt thereof, wherein ring D is a 5- to 6-membered heteroaryl ring.

13. The compound of any one of claims 10-12, or a pharmaceutically acceptable salt thereof, wherein ring A is a monocyclic, bicyclic or tricyclic ring optionally substituted with one or more R^{6a} groups.

14. The compound of any one of claims 10-13, or a pharmaceutically acceptable salt thereof, wherein ring A is C₂₋₁₄heteroaryl optionally substituted with one or more R^{6a} groups.

15. The compound of any one of claims 10-13, or a pharmaceutically acceptable salt thereof, wherein ring A is a phenyl, a pyrimidinyl, a pyridyl, a pyridazinonyl, an 11-15 membered bicyclic heterocyclic ring or an 11-17 membered tricyclic heterocyclic ring, wherein the phenyl, pyrimidinyl, pyridyl, pyridazinonyl, 11-15 membered bicyclic heterocyclic ring and 11-17 membered tricyclic heterocyclic ring are optionally substituted with one or more R^{6a} groups.

16. The compound of any one of claims 10-13, or a pharmaceutically acceptable salt thereof, wherein ring A is C₂₋₁₄heterocycloalkyl optionally substituted with one or more R^{6a} groups.

17. The compound of any one of claims 10-16, or a pharmaceutically acceptable salt thereof, wherein each R^{6a} is independently selected from halogen, C₁₋₆alkyl, C₁₋₆haloalkyl, C₃₋₆cycloalkyl, C₂₋₉heterocycloalkyl, —S(O)₂R¹³, and —S(O)₂N(R¹⁰)(R¹¹)—; wherein C₁₋₆alkyl, C₁₋₆haloalkyl, C₃₋₆ cycloalkyl, and C₂₋₉heterocycloalkyl are optionally substituted with one, two, or three groups selected from R^{9c}.

18. The compound of any one of claims 10-17, or a pharmaceutically acceptable salt thereof, wherein L¹ is —S(O)₂—.

19. The compound of any one of claims 10-17, or a pharmaceutically acceptable salt thereof, wherein L¹ is —C(O)—.

20. The compound of any one of claims 11-19, or a pharmaceutically acceptable salt thereof, wherein L² is a bond.

21. The compound of any one of claims 11-19, or a pharmaceutically acceptable salt thereof, wherein L² is —O—CH₂—.

22. The compound of any one of claims 11-19, or a pharmaceutically acceptable salt thereof, wherein L² is —O—.

23. The compound of any one of claims 1-22, or a pharmaceutically acceptable salt thereof, wherein ring B is a monocyclic ring.

24. The compound of any one of claims 1-22, or a pharmaceutically acceptable salt thereof, wherein ring B is a bicyclic ring.

25. The compound of any one of claims 1-22, or a pharmaceutically acceptable salt thereof, wherein ring B is C₂₋₁₄heterocycloalkyl optionally substituted with one, two, or three groups selected from R^{9b}.

26. The compound of any one of claims 1-22 or 24-25, or a pharmaceutically acceptable salt thereof, wherein ring B is

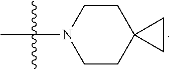 [SMILES: *N1CCC2(CC1)CC2]

27. The compound of any one of claims 1-24, or a pharmaceutically acceptable salt thereof, wherein ring B is C₃₋₁₂cycloalkyl optionally substituted with one, two, or three groups selected from R^{9b}.

28. The compound of claim 27, or a pharmaceutically acceptable salt thereof, wherein ring B is selected from

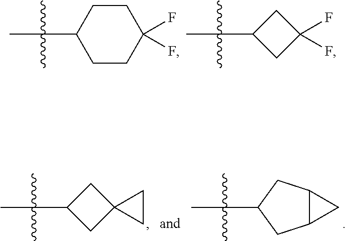 [SMILES: *C1CCC(F)(F)CC1 ; *C1CC(F)(F)C1 ; *C1CC2(CC2)C1 ; *C1CC2CC2C1]

29. The compound of any one of claims 1-28, or a pharmaceutically acceptable salt thereof, wherein R¹ is C₁₋₆ alkyl optionally substituted with one to three substituents selected from —OH and C₁₋₆ alkoxy.

30. The compound of any one of claims 1-28, or a pharmaceutically acceptable salt thereof, wherein R¹ is C₁₋₆alkylene substituted with one —OH group.

31. The compound of any one of claims 1-28, or a pharmaceutically acceptable salt thereof, wherein R¹ is —CH₃, —CH₂CH₂OH or —CH₂CH₂CH₂OH.

32. The compound of any one of claims 1-31, or a pharmaceutically acceptable salt thereof, wherein R³¹ is hydrogen.

33. The compound of any one of claims 1-32, or a pharmaceutically acceptable salt thereof, wherein R³, is hydrogen.

34. The compound of any one of claims 1-33, or a pharmaceutically acceptable salt thereof, wherein R⁴ is hydrogen.

35. A compound, or a pharmaceutically acceptable salt thereof, selected from:

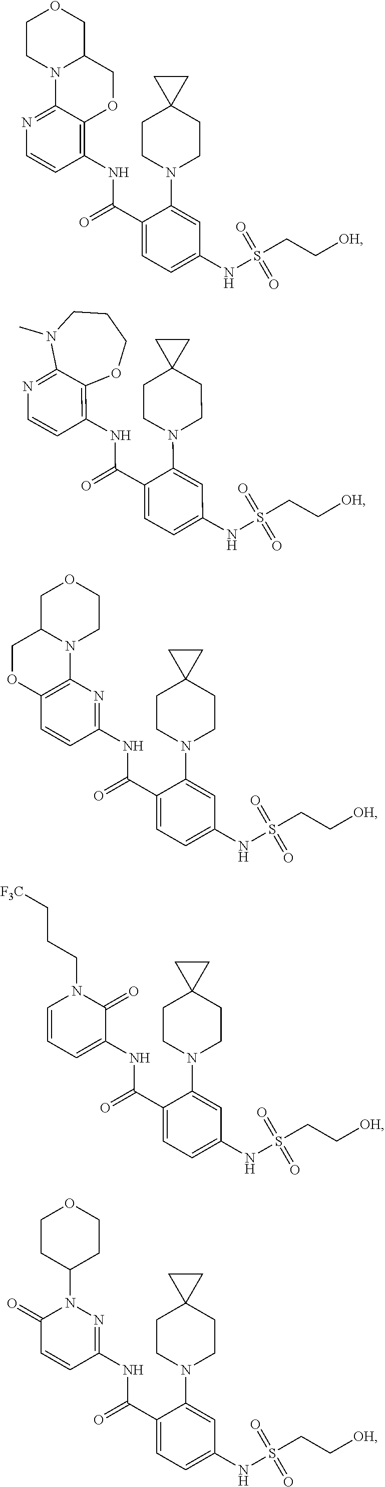 [SMILES: O=C(Nc1ccnc2c1OCC1COCCN21)c1ccc(NS(=O)(=O)CCO)cc1N1CCC2(CC1)CC2 ; CN1CCCOc2c(NC(=O)c3ccc(NS(=O)(=O)CCO)cc3N3CCC4(CC3)CC4)ccnc21 ; O=C(Nc1ccc2c(n1)N1CCOCC1CO2)c1ccc(NS(=O)(=O)CCO)cc1N1CCC2(CC1)CC2 ; O=C(Nc1cccn(CCCC(F)(F)F)c1=O)c1ccc(NS(=O)(=O)CCO)cc1N1CCC2(CC1)CC2 ; O=C(Nc1ccc(=O)n(C2CCOCC2)n1)c1ccc(NS(=O)(=O)CCO)cc1N1CCC2(CC1)CC2]

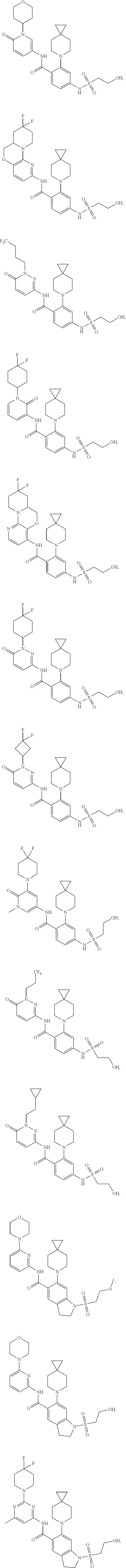

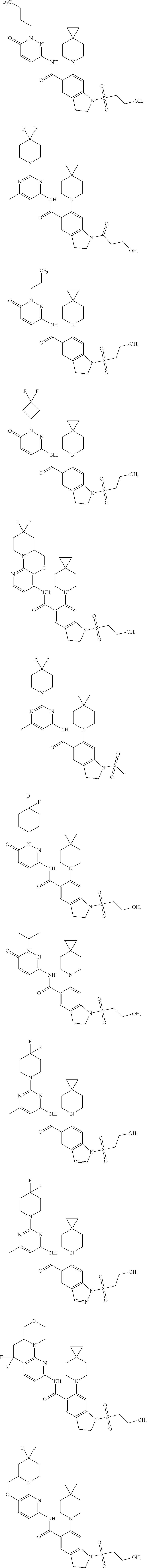

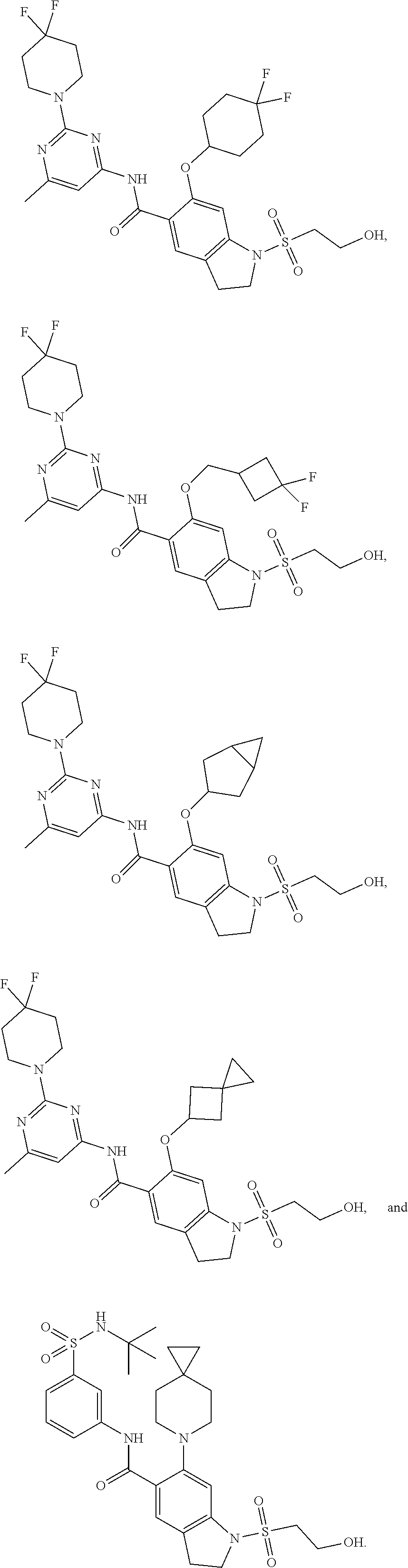 [SMILES: Cc1cc(NC(=O)c2cc3c(cc2OC2CCC(F)(F)CC2)N(S(=O)(=O)CCO)CC3)nc(N2CCC(F)(F)CC2)n1 ; Cc1cc(NC(=O)c2cc3c(cc2OCC2CC(F)(F)C2)N(S(=O)(=O)CCO)CC3)nc(N2CCC(F)(F)CC2)n1 ; Cc1cc(NC(=O)c2cc3c(cc2OC2CC4CC4C2)N(S(=O)(=O)CCO)CC3)nc(N2CCC(F)(F)CC2)n1 ; Cc1cc(NC(=O)c2cc3c(cc2OC2CC4(CC4)C2)N(S(=O)(=O)CCO)CC3)nc(N2CCC(F)(F)CC2)n1 ; CC(C)(C)NS(=O)(=O)c1cccc(NC(=O)c2cc3c(cc2N2CCC4(CC2)CC4)N(S(=O)(=O)CCO)CC3)c1]

36. A pharmaceutical composition comprising a compound of any one of claims 1-35, or a pharmaceutically acceptable salt thereof, and at least one pharmaceutically acceptable excipient.

37. A method of modulating kinase-like protein 18A (KIF18A) in a subject in need thereof, comprising administering to the subject a compound of any one of claims 1-35, or a pharmaceutically acceptable salt thereof, or a pharmaceutical composition of claim 36.

38. A method of inhibiting kinase-like protein 18A (KIF18A) in a subject in need thereof, comprising administering to the subject a compound of any one of claims 1-35, or a pharmaceutically acceptable salt thereof, or a pharmaceutical composition of claim 36.

39. The method of claim 37 or 38, wherein the subject has cancer.

40. A method of treating cancer in a mammal in need thereof, comprising administering to the mammal a compound of any one of claims 1-35, or a pharmaceutically acceptable salt thereof, or a pharmaceutical composition of claim 36.

41. The method of claim 40, wherein the cancer is selected from the group consisting of (a) a solid or hematologically derived tumor selected from cancer of the bladder, endometrial, lung squamous cell, breast, colon, kidney, liver, lung, small cell lung cancer, esophagus, gallbladder, brain, head and neck, ovary, pancreas, stomach, cervix, thyroid, prostate, and skin; (b) a hematopoietic tumor of lymphoid lineage selected from leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma, and Burkett's lymphoma; (c) a hematopoietic tumor of myeloid lineage selected from acute and chronic myelogenous leukemias, myelodysplastic syndrome and promyelocytic leukemia; (d) a tumor of mesenchymal origin selected from fibrosarcoma and rhabdomyosarcoma; (e) a tumor of the central and peripheral nervous system selected from astrocytoma, neuroblastoma, glioma, and schwannoma; and (f) a melanoma, seminoma, teratocarcinoma, osteosarcoma, xenoderoma pigmentosum, keratoctanthoma, thyroid follicular cancer or Karposi's sarcoma.
